(19) United States
(12) Patent Application Publication
(10) Pub. No.: US 2012/0178742 A1
(43) Pub. Date: Jul. 12, 2012

(54) METABOTROPIC GLUTAMATE RECEPTOR MODULATORS

(75) Inventors: Markus Henrich, Munzenberg (DE); Tanja Weil, Ulm (DE); Mirko Hechenberger, Wiesbaden (DE); Sibylle Muller, Toronto (CA); Valerjans Kauss, Riga (LV); Ronalds Zemribo, Jurmala (LV); Elina Erdmane, Riga (LV); Gints Smits, Salacgriva (LV)

(73) Assignee: MERZ PHARMA GmbH & CO. KGaA, FRANKFURT am MAIN (DE)

(21) Appl. No.: 13/388,776

(22) PCT Filed: Aug. 3, 2010
(86) PCT No.: PCT/EP2010/004749

§ 371 Date: Apr. 2, 2012

Related U.S. Application Data
(60) Provisional application No. 61/273,479, filed on Aug. 5, 2009.

(30) Foreign Application Priority Data
Aug. 5, 2009 (EP) 09251944.6

(51) Int. Cl. A61K 31/55

(52) U.S. Cl. 514/217.06

(57) ABSTRACT

The invention relates to heterocyclic derivatives as well as their pharmaceutically acceptable salts. The invention further relates to a process for the preparation of such compounds. The compounds of the invention are mGluR5 modulators and are therefore useful for the control and prevention of acute and/or chronic neurological disorders.

### FIELD OF THE INVENTION

The present invention relates to novel heterocyclic derivatives, which may act as metabotropic glutamate receptor (mGluR) modulators, methods for their synthesis and the treatment and/or prevention of various diseases and disorders, including neurological disorders, by administration of such derivatives.

### BACKGROUND OF THE INVENTION

Neuronal stimuli are transmitted by the central nervous system (CNS) through the interaction of a neurotransmitter released by a neuron, which neurotransmitter has a specific effect on a neuroreceptor of another neuron. L-glutamic acid is considered to be a major excitatory neurotransmitter in the mammalian CNS, consequently playing a critical role in a large number of physiological processes. Glutamate-dependent stimulus receptors are divided into two main groups. The first group comprises ligand-controlled ion channels whereas the other comprises metabotropic glutamate receptors (mGluR). Metabotropic glutamate receptors are a subfamily of G-protein-coupled receptors (GPCR). There is increasing evidence for a peripheral role of both ionotropic and metabotropic glutamate receptors outside the CNS e.g, in chronic pain states.

At present, eight different members of these mGluRs are known. On the basis of structural parameters such as sequence homology, the second messenger system utilized by these receptors and their different affinity to low-molecular weight compounds, these eight receptors may be divided into three groups. MGluR1 and mGluR5 belong to Group I which are positively coupled to phospholipase C and their activation leads to a mobilization of intracellular calcium ions. MGluR2 and mGluR3 belong to Group II and mGluR4, mGluR6, mGluR7 and mGluR8 belong to Group III, both of which are negatively coupled to adenylyl cyclase, i.e., their activation causes a reduction in second messenger cAMP and thus a dampening of neuronal activity.

The mGluR5 modulators have been shown to modulate the effects of the presynaptically released neurotransmitter glutamate via postsynaptic mechanisms (receptors). Moreover, as these modulators may be both positive and/or negative mGluR5 modulators, such modulators may increase or inhibit the effects mediated through these metabotropic glutamate receptors.

Modulators which are negative mGluR5 modulators decrease the effects mediated through metabotropic glutamate receptors. Since a variety of patho-physiological processes and disease states affecting the CNS are thought to be related to abnormal glutamate neurotransmission, and mGluR5 receptors are shown to be expressed in many areas of the CNS and in PNS (peripheral nervous system), modulators of these receptors could be therapeutically beneficial in the treatment of diseases involving CNS and PNS.

Therefore, mGluR5 positive or negative modulators may be administered to provide neuroprotection and/or disease modification in the following acute or chronic pathological conditions or to provide a symptomatological effect on the following conditions: Alzheimer's disease, Creutzfeld-Jakob's syndrome/disease, bovine spongiform encephalopathy (BSE), prion related infections, diseases involving mitochondrial dysfunction, diseases involving β-amyloid and/or tauopathy, Down's syndrome, hepatic encephalopathy, Huntington's disease, motor neuron diseases, amyotrophic lateral sclerosis (ALS), olivoponto-cerebellar atrophy, post-operative cognitive deficit (POCD), systemic lupus erythematosus, systemic sclerosis, Sjogren's syndrome, Neuronal Ceroid Lipofuscinosis, neurodegenerative cerebellar ataxias, Parkinson's disease, Parkinson's dementia, mild cognitive impairment, cognitive deficits in various forms of mild cognitive impairment, cognitive deficits in various forms of dementia, dementia pugilistica, vascular and frontal lobe dementia, cognitive impairment, learning impairment, eye injuries, eye diseases, eye disorders, glaucoma, retinopathy, macular degeneration, head or brain or spinal cord injuries, head or brain or spinal cord trauma, trauma, hypoglycaemia, hypoxia, perinatal hypoxia, ischaemia, ischaemia resulting from cardiac arrest or stroke or bypass operations or transplants, convulsions, epileptic convulsions, epilepsy, temporal lobe epilepsy, myoclonic epilepsy, inner ear insult, inner ear insult in tinnitus, tinnitus, sound- or drug-induced inner ear insult, sound- or drug-induced tinnitus, hyperacusis, L-dopa-induced dykinesias, L-dopa-induced dykinesias in Parkinson's disease therapy, dyskinesias, dyskinesia in Huntington's disease, drug induced dyskinesias, neuroleptic-induced dyskinesias, haloperidol-induced dyskinesias, dopaminomimetic-induced dyskinesias, chorea, Huntington's chorea, athetosis, dystonia, stereotypy, ballism, tardive dyskinesias, neuroleptics-induced dyskinesia, tic disorder, torticollis spasmodicus, blepharospasm, focal and generalized dystonia, nystagmus, hereditary cerebellar ataxias, corticobasal degeneration, tremor, essential tremor, abuse, addiction, nicotine addiction, nicotine abuse, alcohol addiction, alcohol abuse, opiate addiction, opiate abuse, cocaine addiction, cocaine abuse, amphetamine addiction, amphetamine abuse, anxiety disorders, panic disorders, anxiety and panic disorders, social anxiety disorder (SAD), attention deficit hyperactivity disorder (ADHD), attention deficit syndrome (ADS), restless leg syndrome (RLS), hyperactivity in children, autism, dementia, dementia in Alzheimer's disease, dementia in Korsakoff syndrome, Korsakoff syndrome, vascular dementia, dementia related to HIV infections, HIV-1 encephalopathy, AIDS encephalopathy, AIDS dementia complex, AIDS-related dementia, major depressive disorder, major depression, depression, depression resulting from Borna virus infection, major depression resulting from Borna virus infection, bipolar manic-depressive disorder, drug tolerance, drug tolerance to opioids, movement disorders, fragile-X syndrome, irritable bowel syndrome (IBS), migraine, multiple sclerosis (MS), muscle spasms, pain, chronic pain, acute pain, inflammatory pain, neuropathic pain, diabetic neuropathic pain (DNP), pain related to rheumatic arthritis, allodynia, hyperalgesia, nociceptive pain, cancer pain, posttraumatic stress disorder (PTSD), schizophrenia, positive or cognitive or negative symptoms of schizophrenia, spasticity, Tourette's syndrome, urinary incontinence, vomiting, pruritic conditions, pruritis, sleep disorders, micturition disorders, neuromuscular disorder in the lower urinary tract, gastroesophageal reflux disease (GERD), gastrointestinal dysfunction, lower esophageal sphincter (LES) disease, functional gastrointestinal disorders, dyspepsia, regurgitation, respiratory tract infection, bulimia nervosa, chronic laryngitis, asthma, reflux-related asthma, lung disease, eating disorders, obesity, obesity-related disorders, obesity abuse, food addiction, binge eating disorders, agoraphobia, generalized anxiety disorder, obsessive-compulsive disorder, panic disorder, posttraumatic stress disorder, social phobia, phobic disorders, substance-induced anxiety disorder, delusional disorder, schizoaffective disorder, schizophreniform disorder, substance-induced psychotic disorder, or delirium, diabetes, hyperammonemia and liver failure and sleep disturbances.

MGluR5 negative or positive modulators may also be administered to provide inhibition of tumour cell growth, migration, invasion, adhesion and toxicity in the peripheral tissues, peripheral nervous system and CNS. MGluR5 modulators may be administered to provide therapeutic intervention in neoplasia, hyperplasia, dysplasia, cancer, carcinoma, sarcoma, oral cancer, squamous cell carcinoma (SCC), oral squamous cell carcinoma (SCC), lung cancer, lung adenocarcinoma, breast cancer, prostate cancer, gastric cancer, liver cancer, colon cancer, colorectal carcinoma, rhabdomyosarcoma, brain tumour, tumour of a nerve tissue, glioma, malignant glioma, astroglioma, neuroglioma, neuroblastoma, glioblastoma, medulloblastoma, cancer of skin cells, melanoma, malignant melanoma, epithelial neoplasm, lymphoma, myeloma, Hodgkin's disease, Burkitt's lymphoma, leukemia, thymoma, and other tumours.

MGluR5 positive or negative modulators may also be administered to provide disease modification and/or to provide a symptomatological effect on the following conditions: diabetes, hyperammonemia and liver failure.

Further indications for mGluR5 negative or positive modulators include those indications wherein a particular condition does not necessarily exist but wherein a particular physiological parameter may be improved through administration of the instant compounds, for example cognitive enhancement, learning impairment and/or neuroprotection.

Positive modulators may be particularly useful in the treatment of positive and negative symptoms in schizophrenia and cognitive deficits in various forms of dementia and mild cognitive impairment (Kinney et al. The Journal of Pharmacology and Experimental Therapeutics, 2005, 313 199-206).

Moreover, mGluR modulators may have activity when administered in combination with other substances exhibiting neurological effects via different mechanisms.

Simultaneous administration of Group I mGluR modulators and NMDA receptor antagonists has also been shown to provide neuroprotection in animal models (Zieminska et al. Acta Neurobiol. Exp., 2006, 66, 301-309; Zieminska et al. Neurochemistry International, 2003, 43, 481-492; and Zieminska et al. Neurochemistry International, 2006, 48, 491-497).

Simultaneous administration of Group I mGluR modulators and compounds such as L-DOPA, dopaminomimetics, and/or neuroleptics may be useful in treating various conditions including drug induced dyskinesias, neuroleptic-induced dyskinesias, haloperidol-induced dyskinesias, dopaminomimetic-induced dyskinesias.

Furthermore, it has also been hypothesized that drugs which possess activity at multiple targets may be useful in treating neurodegenerative diseases, including Alzheimer's disease, Parkinson's disease and their associated symptoms (Cavalli, et al., J. Med. Chem., 2008, 51, 347-372 and Morphy, et al., J. Med. Chem., 2005, 48, 6523-6543).

Compounds which possess monoamine oxidase B (MAO-B) inhibitory activity have been disclosed to be useful in treating neurodegenerative diseases such as Alzheimer's disease and Parkinson's disease (Santana, et al., J. Med. Chem., 2008, 51, 6740-6751). It has been reported that preliminary data suggest that MAO inhibition may represent an interesting property to consider when designing multi-target-directed ligands (MTDLs) for Alzheimer's disease. Moreover, MTDLs for Parkinson's disease have been based on MAO inhibition in combination with a second activity (Cavalli, et al., J. Med. Chem., 2008, 51, 347-372).

Thus, mGluR modulators which also possess MAO-B inhibitory activity may be particularly useful in treating neurodegenerative diseases such as Alzheimer's disease and Parkinson's disease and their associated symptoms.

Modulators of mGluR5 have been previously described. Iso, et al. (J. Med. Chem., 2006, 49, 1080-1100) disclose MTEP analogues and their activity as mGluR5 antagonists which may be useful in the treatment of drug addiction.

### THE PRESENT INVENTION

It now has been found that certain heterocyclic derivatives are potent mGluR5 modulators. Additionally, these heterocyclic derivatives may also exhibit MAO-B inhibitory activity. Therefore, these substances may be therapeutically beneficial in the treatment of conditions which involve abnormal glutamate neurotransmission or in which modulation of mGluR5 receptors results in therapeutic benefit and/or in the treatment of conditions in which MAO-B plays a role. These substances may be administered in the form of a pharmaceutical composition, wherein they are present together with one or more pharmaceutically acceptable diluents, carriers, or excipients.

### OBJECTS OF THE INVENTION

It is an object of the present invention to provide novel pharmaceutical compounds which are mGluR5 modulators and pharmaceutical compositions thereof. It is a further object of the invention to provide a novel method of treating, eliminating, alleviating, palliating, modifying, or ameliorating undesirable CNS disorders which involve abnormal glutamate neurotransmission and/or CNS disorders involving MAO-B, and/or to provide symptomological effects, by employing a compound of the invention or a pharmaceutical composition containing the same.

An additional object of the invention is the provision of processes for producing the heterocyclic derivatives.

### SUMMARY OF THE INVENTION

What we therefore believe to be comprised by our invention may be summarized inter alia in the following words:

A compound selected from those of Formula I

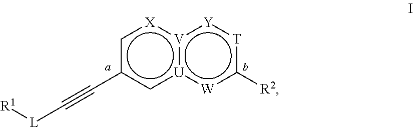

wherein
L represents a bond or CH₂;
T represents a bond or CH;
U and V represent C or N;
W represents N, O, or S;
X represents CH or N;
Y represents CH, N, or N—R⁵, wherein R⁵ represents C₁₋₆alkyl;
it being understood that the valency of the atoms is respected and that the variables a and b represent the points of attachment for the

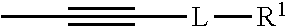

moiety and the R² substituent, respectively;
R¹ represents aryl, heteroaryl, cycloC₃₋₁₂alkyl, cycloC₃₋₁₂alkenyl, or heterocyclyl;
R² represents hydrogen, C₁₋₆alkyl, C₁₋₆alkoxycarbonyl, cycloC₃₋₁₂alkoxycarbonyl, aryl, heteroaryl, C₁₋₆alkoxy, —NR³R⁴, or —C(O)NR³R⁴, wherein R³ and R⁴, which may be the same or different, each independently represent hydrogen, C₁₋₆alkyl, or cycloC₃₋₁₂alkyl, or R³ and R⁴, together with the nitrogen atom to which they are attached, represent a 5-, 6-, or 7-membered ring which may be saturated or unsaturated, wherein the ring in addition to the nitrogen atom may contain an additional heteroatom selected from sulfur, oxygen and nitrogen and/or be optionally fused to a benzene ring, and wherein the ring may be optionally substituted by one or more substituents selected from C₁₋₆alkyl, halogen, trifluoromethyl, C₁₋₆alkoxy, hydroxy, cyano, oxo, and phenyl;
wherein the term “aryl” means phenyl or naphthyl, wherein the phenyl or naphthyl group is optionally substituted by one or more substituents, which may be the same or different, selected independently from halogen, trifluoromethyl, trifluoromethoxy, C₁₋₆alkyl, hydroxyC₁₋₆alkyl, C₂₋₆alkenyl, C₁₋₆alkoxy, C₁₋₆alkoxyC₁₋₆alkyl, amino, hydroxy, nitro, cyano, formyl, cyanomethyl, C₁₋₆alkoxycarbonyl, C₁₋₆alkylcarbonyloxy, C₁₋₆alkylcarbonyloxyC₁₋₆alkyl, C₁₋₆alkylamino, di-(C₁₋₆alkyl)amino, C₁₋₆alkylcarbonylamino, phenylcarbonylamino, aminocarbonyl, N—C₁₋₆alkylaminocarbonyl, di-N,N—C₁₋₆alkylaminocarbonyl, pyrrolidinyl, piperidinyl, morpholinyl, piperazinyl, cycloC₃₋₁₂alkyl and optionally C₁₋₆alkylenedioxy;
the term “heteroaryl” means an aromatic 5-6 membered ring containing from one to four heteroatoms selected from oxygen, sulfur and nitrogen, or a bicyclic group comprising a 5-6 membered ring containing from one to four heteroatoms selected from oxygen, sulfur and nitrogen fused with a benzene ring or a 5-6 membered ring containing from one to four heteroatoms selected from oxygen, sulfur and nitrogen, wherein the heteroaryl group may be optionally substituted by one or more substituents, which may be the same or different, selected independently from halogen, trifluoromethyl, trifluoromethoxy, C₁₋₆alkyl, hydroxyC₁₋₆alkyl, C₂₋₆alkenyl, C₁₋₆alkoxy, amino, hydroxy, nitro, cyano, C₁₋₆alkoxycarbonyl, C₁₋₆alkoxycarbonyloxy, C₁₋₆alkylamino, di-(C₁₋₆alkyl)amino, C₁₋₆alkylcarbonylamino, aminocarbonyl, N—C₁₋₆alkylaminocarbonyl, di-N,N—C₁₋₆alkylaminocarbonyl, pyrrolidinyl, piperidinyl, morpholinyl, cycloC₃₋₁₂alkyl, C₁₋₆alkylenedioxy and aryl;
   and optical isomers, prodrugs, pharmaceutically acceptable salts, hydrates, solvates, and polymorphs thereof;
   it being understood that:
   if T represents CH, then W and X each represent N;
   if T represents a bond, then at least one of U or X represents N;
   if T represents a bond and W, U, and X all represent N, then R¹ may not represent cycloC₃₋₁₂alkyl or saturated heterocyclyl;
   R¹ may not represent quinazoline;
   and
   the compound of Formula I may not represent
6-[2-(3-fluorophenylethynyl)]-[1,2,4]triazolo[1,5-a]pyridine,
6-[2-(3-nitrophenylethynyl)]-[1,2,4]triazolo[1,5-a]pyridine,
6-[2-(3-methylphenylethynyl)]-[1,2,4]triazolo[1,5-a]pyridine,
6-[2-(4-chlorophenylethynyl)]-[1,2,4]triazolo[1,5-a]pyridine,
6-[2-(4-fluorophenylethynyl)]-[1,2,4]triazolo[1,5-a]pyridine,
6-[2-(4-methylphenylethynyl)]-[1,2,4]triazolo[1,5-a]pyridine,
6-[2-(3,4-difluorophenylethynyl)]-[1,2,4]triazolo[1,5-a]pyridine,
6-[2-(2-chlorophenylethynyl)]-[1,2,4]triazolo[1,5-a]pyridine,
6-[2-(3-chlorophenylethynyl)]-[1,2,4]triazolo[1,5-a]pyridine,
6-[2-(4-methyl-2-thiazolyl)ethynyl]-[1,2,4]triazolo[1,5-a]pyridine, or
6-[2-(6-methyl-2-pyridinyl)ethynyl]-[1,2,4]triazolo[1,5-a]pyridine.

Such a compound of Formula I, wherein the ring represented by

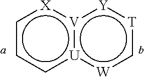

is selected from:

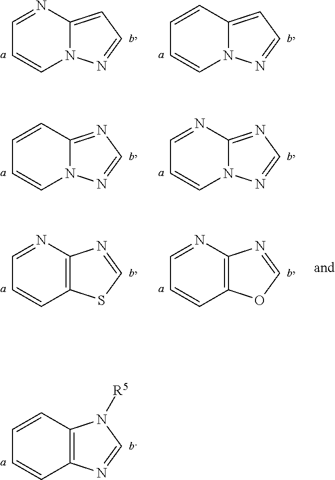

Such a compound of Formula I, wherein R⁵ represents methyl.

Such a compound of Formula I, wherein the ring represented by

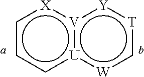

is selected from:

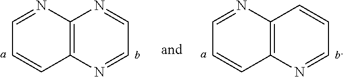

Such a compound of Formula I, wherein R¹ represents aryl, heteroaryl, cycloC₃₋₁₂alkenyl, or heterocyclyl.

Such a compound of Formula I, wherein R¹ represents phenyl which is optionally substituted by one or more substituents selected from halogen, C₁₋₆alkyl, hydroxyl, and trifluoromethyl; thiophenyl which is optionally substituted by one or more C₁₋₆alkyl groups; cyclohexenyl; dihydrothiopyran; dihydropyridine which may be optionally substituted by one or more C₁₋₆alkoxycarbonyl groups; dihydropyran; pyridine which may be optionally substituted by one or more substituents selected from amino and C₁₋₆alkylamino; or pyrimidine which may be optionally substituted by one or more C₁₋₆alkylamino groups.

Such a compound of Formula I, wherein R² represents hydrogen, aryl, heteroaryl, C₁₋₆alkoxy, or —NR³R⁴, or —C(O)NR³R⁴, wherein R³ and R⁴ together with the nitrogen atom to which they are attached, represent a 5-, 6-, or 7-membered ring which may be saturated or unsaturated, wherein the ring in addition to the nitrogen atom may contain an additional heteroatom selected from sulfur, oxygen and nitrogen and/or be optionally fused to a benzene ring, and wherein the ring may be optionally substituted by one or more substituents selected from C₁₋₆alkyl, hydroxy, oxo, and phenyl.

Such a compound of Formula I, wherein R² represents hydrogen, phenyl which is optionally substituted by one or more halogen atoms, piperidino, methoxy, furanyl, or —C(O)NR³R⁴, wherein R³ and R⁴ together with the nitrogen atom to which they are attached, represent a ring selected from morpholine, piperidine, pyrrolidine, azepine, and 1,3-dihydro-isoindole, wherein the ring may be optionally substituted by one or more substituents selected from methyl, hydroxy, oxo, and phenyl.

A further aspect of the invention relates to a compound of Formula I, which is selected from those of Formula IA:

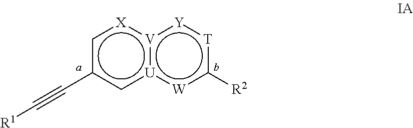

wherein R¹, R², T, U, V, X, Y, and W are as defined above for Formula I.

A further aspect of the invention relates to a compound of Formula I, which is selected from those of Formula IB:

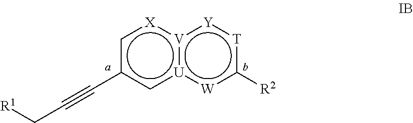

wherein R¹, R², T, U, V, X, Y, and W are as defined above for Formula I.

A further aspect of the invention relates to a compound of Formula I, which is selected from those of Formula IC:

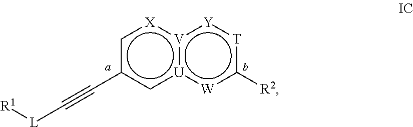

wherein
L represents a bond or CH₂;
T represents a bond or CH;
U and V represent C or N;
W represents N, O, or S;
X and Y represent CH or N;
it being understood that the valency of the atoms is respected and that the variables a and b represent the points of attachment for the

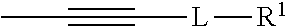

R¹ represents aryl, heteroaryl, cycloC₃₋₁₂alkyl, cycloC₃₋₁₂alkenyl, or heterocyclyl;
R² represents hydrogen, C₁₋₆alkyl, C₁₋₆alkoxycarbonyl, cycloC₃₋₁₂alkoxycarbonyl, aryl, heteroaryl, or, —C(O)NR³R⁴, wherein R³ and R⁴, which may be the same or different, each independently represent hydrogen, C₁₋₆alkyl, or cycloC₃₋₁₂alkyl, or R³ and R⁴, together with the nitrogen atom to which they are attached, represent a 5-, 6-, or 7-membered ring which may be saturated or unsaturated, wherein the ring in addition to the nitrogen atom may contain an additional heteroatom selected from sulfur, oxygen and nitrogen and/or be optionally fused to a benzene ring, and wherein the ring may be optionally substituted by one or more substituents selected from C₁₋₆alkyl, halogen, trifluoromethyl, C₁₋₆alkoxy, hydroxy, cyano, oxo, and phenyl;
wherein the term “aryl” means phenyl or naphthyl, wherein the phenyl or naphthyl group is optionally substituted by one or more substituents, which may be the same or different, selected independently from halogen, trifluoromethyl, trifluoromethoxy, C₁₋₆alkyl, hydroxyC₁₋₆alkyl, C₂₋₆alkenyl, C₁₋₆alkoxy, C₁₋₆alkoxyC₁₋₆alkyl, amino, hydroxy, nitro, cyano, formyl, cyanomethyl, C₁₋₆alkoxycarbonyl, C₁₋₆alkylcarbonyloxy, C₁₋₆alkylcarbonyloxyC₁₋₆alkyl, C₁₋₆alkylamino, di-(C₁₋₆alkyl)amino, C₁₋₆alkyl-carbonylamino, phenylcarbonylamino, aminocarbonyl, N—C₁₋₆alkylaminocarbonyl, di-N,N—C₁₋₆alkylaminocarbonyl, pyrrolidinyl, piperidinyl, morpholinyl, piperazinyl, cycloC₃₋₁₂alkyl and optionally C₁₋₆alkylenedioxy;
the term “heteroaryl” means an aromatic 5-6 membered ring containing from one to four heteroatoms selected from oxygen, sulfur and nitrogen, or a bicyclic group comprising a 5-6 membered ring containing from one to four heteroatoms selected from oxygen, sulfur and nitrogen fused with a benzene ring or a 5-6 membered ring containing from one to four heteroatoms selected from oxygen, sulfur and nitrogen, wherein the heteroaryl group may be optionally substituted by one or more substituents, which may be the same or different, selected independently from halogen, trifluoromethyl, trifluoromethoxy, C₁₋₆alkyl, hydroxyC₁₋₆alkyl, C₂₋₆alkenyl, C₁₋₆alkoxy, amino, hydroxy, nitro, cyano, C₁₋₆alkoxycarbonyl, C₁₋₆alkoxycarbonyloxy, C₁₋₆alkylamino, and di-(C₁₋₆ alkyl)amino, C₁₋₆alkylcarbonylamino, aminocarbonyl, N—C₁₋₆alkylaminocarbonyl, di-N,N—C₁₋₆alkylaminocarbonyl, pyrrolidinyl, piperidinyl, morpholinyl, cycloC₃₋₁₂alkyl, C₁₋₆alkylenedioxy and aryl;
and optical isomers, prodrugs, pharmaceutically acceptable salts, hydrates, solvates, and polymorphs thereof;
it being understood that:
if T represents CH, then W and X each represent N;
if T represents a bond, then at least one of U or X represents N;
if T represents a bond and W, U, and X all represent N, then R¹ may not represent cycloC₃₋₁₂alkyl or saturated heterocyclyl;
R¹ may not represent quinazoline;
and
the compound of Formula I may not represent
6-[2-(3-fluorophenylethynyl)]-[1,2,4]triazolo[1,5-a]pyridine,
6-[2-(3-nitrophenylethynyl)]-[1,2,4]triazolo[1,5-a]pyridine,
6-[2-(3-methylphenylethynyl)]-[1,2,4]triazolo[1,5-a]pyridine,
6-[2-(4-chlorophenylethynyl)]-[1,2,4]triazolo[1,5-a]pyridine,
6-[2-(4-fluorophenylethynyl)]-[1,2,4]triazolo[1,5-a]pyridine,
6-[2-(4-methylphenylethynyl)]-[1,2,4]triazolo[1,5-a]pyridine,
6-[2-(3,4-difluorophenylethynyl)]-[1,2,4]triazolo[1,5-a]pyridine,
6-[2-(2-chlorophenylethynyl)]-[1,2,4]triazolo[1,5-a]pyridine,
6-[2-(3-chlorophenylethynyl)]-[1,2,4]triazolo[1,5-a]pyridine,
6-[2-(4-methyl-2-thiazolyl)ethynyl]-[1,2,4]triazolo[1,5-a]pyridine, or
6-[2-(6-methyl-2-pyridinyl)ethynyl]-[1,2,4]triazolo[1,5-a]pyridine.

Such a compound of Formula IC, wherein the ring represented by

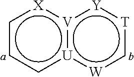

is selected from:

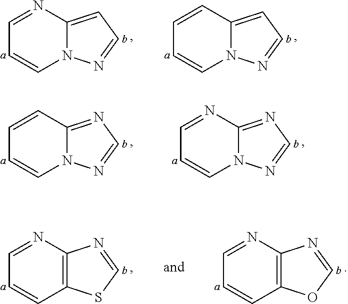

Such a compound of Formula IC, wherein the ring represented by

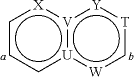

is selected from:

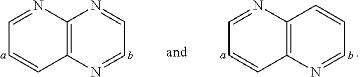

Such a compound of Formula IC, wherein R¹ represents aryl, heteroaryl, cycloC₃₋₁₂alkenyl, or heterocyclyl.

Such a compound of Formula IC, wherein R¹ represents phenyl which is optionally substituted by one or more substituents selected from halogen and C₁₋₆alkyl; thiophenyl which is optionally substituted by one or more C₁₋₆alkyl groups; cyclohexenyl; dihydrothiopyran; or dihydropyridine which may be optionally substituted by one or more C₁₋₆alkoxycarbonyl groups.

Such a compound of Formula IC, wherein R² represents hydrogen, aryl, or —C(O)NR³R⁴, wherein R³ and R⁴ together with the nitrogen atom to which they are attached, represent a 5-, 6-, or 7-membered ring which may be saturated or unsaturated, wherein the ring in addition to the nitrogen atom may contain an additional heteroatom selected from sulfur, oxygen and nitrogen and/or be optionally fused to a benzene ring, and wherein the ring may be optionally substituted by one or more substituents selected from C₁₋₆alkyl, hydroxy, oxo, and phenyl.

Such a compound of Formula IC, wherein R² represents hydrogen, phenyl which is optionally substituted by one or more halogen atoms, or —C(O)NR³R⁴, wherein R³ and R⁴ together with the nitrogen atom to which they are attached, represent a ring selected from morpholine, piperidine, pyrrolidine, azepine, and 1,3-dihydro-isoindole, wherein the ring may be optionally substituted by one or more substituents selected from methyl, hydroxy, oxo, and phenyl.

Specific compounds of Formula I within the present invention include, but are not limited to, the following compounds:
6-Phenylethynyl-pyrazolo[1,5-a]pyrimidine,
6-(3,5-Dichloro-phenylethynyl)-pyrazolo[1,5-a]pyrimidine,
6-(3-Fluoro-phenylethynyl)-pyrazolo[1,5-a]pyrimidine,
6-(4-Fluoro-phenylethynyl)-pyrazolo[1,5-a]pyrimidine,
6-(2-Fluoro-phenylethynyl)-pyrazolo[1,5-a]pyrimidine,
6-Thiophen-3-ylethynyl-pyrazolo[1,5-a]pyrimidine,
6-(3-Methyl-thiophen-2-ylethynyl)-pyrazolo[1,5-a]pyrimidine,
6-Cyclohex-1-enylethynyl-pyrazolo[1,5-a]pyrimidine,
6-p-Tolylethynyl-pyrazolo[1,5-a]pyrimidine,
6-(3,6-Dihydro-2H-thiopyran-4-ylethynyl)-pyrazolo[1,5-a]pyrimidine,
6-(3,5-Difluoro-phenylethynyl)-pyrazolo[1,5-a]pyrimidine,
4-Pyrazolo[1,5-a]pyrimidin-6-ylethynyl-3,6-dihydro-2H-pyridine-1-carboxylic acid tert-butyl ester,
6-Thiophen-2-ylethynyl-pyrazolo[1,5-a]pyrimidine,
6-(3-Phenyl-prop-1-ynyl)-pyrazolo[1,5-a]pyrimidine,
Morpholin-4-yl-(6-phenylethynyl-pyrazolo[1,5-a]pyrimidin-2-yl)-methanone,
(6-Phenylethynyl-pyrazolo[1,5-a]pyrimidin-2-yl)-piperidin-1-yl-methanone,
Azepan-1-yl-(6-phenylethynyl-pyrazolo[1,5-a]pyrimidin-2-yl)-methanone,
(6-Phenylethynyl-pyrazolo[1,5-a]pyrimidin-2-yl)-(4-phenyl-piperidin-1-yl)-methanone,
(6-Phenylethynyl-pyrazolo[1,5-a]pyrimidin-2-yl)-pyrrolidin-1-yl-methanone,
(1,3-Dihydro-isoindol-2-yl)-(6-phenylethynyl-pyrazolo[1,5-a]pyrimidin-2-yl)-methanone,
1-(6-Phenylethynyl-pyrazolo[1,5-a]pyrimidine-2-carbonyl)-piperidin-4-one,
4-[2-(Piperidine-1-carbonyl)-pyrazolo[1,5-a]pyrimidin-6-ylethynyl]-3,6-dihydro-2H-pyridine-1-carboxylic acid tert-butyl ester,
(4-Hydroxy-4-methyl-piperidin-1-yl)-(6-phenylethynyl-pyrazolo[1,5-a]pyrimidin-2-yl)-methanone,
(4-Hydroxy-piperidin-1-yl)-(6-phenylethynyl-pyrazolo[1,5-a]pyrimidin-2-yl)-methanone,
(1-Methyl-3,4-dihydro-1H-isoquinolin-2-yl)-(6-phenylethynyl-pyrazolo[1,5-a]pyrimidin-2-yl)-methanone,
6-Phenylethynyl-pyrazolo[1,5-a]pyrimidine-2-carboxylic acid cyclohexylamide,
6-Phenylethynyl-pyrazolo[1,5-a]pyrimidine-2-carboxylic acid cyclopentylamide,
2-(4-Fluoro-phenyl)-6-phenylethynyl-pyrazolo[1,5-a]pyrimidine,
6-Phenylethynyl-pyrazolo[1,5-a]pyridine,
6-Cyclohex-1-enylethynyl-pyrazolo[1,5-a]pyridine,
6-p-Tolylethynyl-pyrazolo[1,5-a]pyridine,
(6-Phenylethynyl-pyrazolo[1,5-a]pyridin-2-yl)-piperidin-1-yl-methanone,
6-Phenylethynyl-[1,2,4]triazolo[1,5-a]pyrimidine,
6-Thiophen-2-ylethynyl-[1,2,4]triazolo[1,5-a]pyrimidine,
6-p-Tolylethynyl-[1,2,4]triazolo[1,5-a]pyrimidine,
(6-Phenylethynyl-[1,2,4]triazolo[1,5-a]pyrimidin-2-yl)-piperidin-1-yl-methanone,
6-Phenylethynyl-[1,2,4]triazolo[1,5-a]pyridine,
6-Phenylethynyl-thiazolo[4,5-b]pyridine,
7-Phenylethynyl-pyrido[2,3-b]pyrazine,
7-Cyclohex-1-enylethynyl-pyrido[2,3-b]pyrazine,
3-Phenylethynyl-[1,5]naphthyridine,
6-Phenylethynyl-oxazolo[4,5-b]pyridine,
(6-Phenylethynyl-oxazolo[4,5-b]pyridin-2-yl)-piperidin-1-yl-methanone,
6-(3-Fluoro-phenylethynyl)-thiazolo[4,5-b]pyridine,
6-(2-Fluoro-phenylethynyl)-thiazolo[4,5-b]pyridine,
6-(4-Fluoro-phenylethynyl)-thiazolo[4,5-b]pyridine,
6-(3-Fluoro-phenylethynyl)-[1,2,4]triazolo[1,5-a]pyrimidine,
6-(2-Fluoro-phenylethynyl)-[1,2,4]triazolo[1,5-a]pyrimidine,
6-(4-Fluoro-phenylethynyl)-[1,2,4]triazolo[1,5-a]pyrimidine,
[6-(3-Fluoro-phenylethynyl)-[1,2,4]triazolo[1,5-a]pyrimidin-2-yl]-piperidin-1-yl-methanone,
[6-(2-Fluoro-phenylethynyl)-[1,2,4]triazolo[1,5-a]pyrimidin-2-yl]-piperidin-1-yl-methanone,
[6-(4-Fluoro-phenylethynyl)-[1,2,4]triazolo[1,5-a]pyrimidin-2-yl]-piperidin-1-yl-methanone,
6-(3-Fluoro-phenylethynyl)-[1,2,4]triazolo[1,5-a]pyridine,
6-(2-Fluoro-phenylethynyl)-[1,2,4]triazolo[1,5-a]pyridine,
6-(4-Fluoro-phenylethynyl)-[1,2,4]triazolo[1,5-a]pyridine,
[6-Phenylethynyl-[1,2,4]triazolo[1,5-a]pyridin-2-yl]-piperidin-1-yl-methanone,
[6-(3-Fluoro-phenylethynyl)-[1,2,4]triazolo[1,5-a]pyridin-2-yl]-piperidin-1-yl-methanone,
[6-(2-Fluoro-phenylethynyl)-[1,2,4]triazolo[1,5-a]pyridin-2-yl]-piperidin-1-yl-methanone,
[6-(4-Fluoro-phenylethynyl)-[1,2,4]triazolo[1,5-a]pyridin-2-yl]-piperidin-1-yl-methanone,
7-(3-Fluoro-phenylethynyl)-pyrido[2,3-b]pyrazine,
7-(2-Fluoro-phenylethynyl)-pyrido[2,3-b]pyrazine,
7-(4-Fluoro-phenylethynyl)-pyrido[2,3-b]pyrazine,
[7-phenylethynyl-pyrido[2,3-b]pyrazin-3-yl]-piperidin-1-yl-methanone,
[7-(3-Fluoro-phenylethynyl)-pyrido[2,3-b]pyrazin-3-yl]-piperidin-1-yl-methanone,
[7-(2-Fluoro-phenylethynyl)-pyrido[2,3-b]pyrazin-3-yl]-piperidin-1-yl-methanone,
[7-(4-Fluoro-phenylethynyl)-pyrido[2,3-b]pyrazin-3-yl]-piperidin-1-yl-methanone,
3-(3-Fluoro-phenylethynyl)-[1,5]naphthyridine,
3-(2-Fluoro-phenylethynyl)-[1,5]naphthyridine,
3-(4-Fluoro-phenylethynyl)-[1,5]naphthyridine,
6-(4-Fluoro-phenylethynyl)-oxazolo[4,5-b]pyridine,
6-Cyclohex-1-enylethynyl-oxazolo[4,5-b]pyridine,
(6-Cyclohex-1-enylethynyl-oxazolo[4,5-b]pyridin-2-yl)-piperidin-1-yl-methanone,
6-(m-Tolylethynyl)thiazolo[4,5-b]pyridine,
6-(p-Tolylethynyl)thiazolo[4,5-b]pyridine,
6-(o-Tolylethynyl)thiazolo[4,5-b]pyridine,
6-(Pyridin-4-ylethynyl)thiazolo[4,5-b]pyridine,
6-(Pyridin-3-ylethynyl)thiazolo[4,5-b]pyridine,
6-((2,6-Difluorophenyl)ethynyl)thiazolo[4,5-b]pyridine,
6-((2,4-Difluorophenyl)ethynyl)thiazolo[4,5-b]pyridine,
6-((3,5-Difluorophenyl)ethynyl)thiazolo[4,5-b]pyridine,
6-Phenylethynyl-2-piperidin-1-yl-thiazolo[4,5-b]pyridine,
6-(p-Tolylethynyl)-[1,2,4]triazolo[1,5-a]pyridine,
6-(o-Tolylethynyl)-[1,2,4]triazolo[1,5-a]pyridine,
2-Furan-2-yl-6-phenylethynyl-[1,2,4]triazolo[1,5-a]pyridine,
7-(p-Tolylethynyl)-pyrido[2,3-b]pyrazine,
7-(m-Tolylethynyl)-pyrido[2,3-b]pyrazine,
7-(o-Tolylethynyl)-pyrido[2,3-b]pyrazine,
7-(Pyridin-4-ylethynyl)pyrido[2,3-b]pyrazine,
7-(Pyridin-3-ylethynyl)pyrido[2,3-b]pyrazine,
4-(Pyrido[2,3-b]pyrazin-7-ylethynyl)phenol,
7-((3,6-Dihydro-2H-pyran-4-yl)ethynyl)pyrido[2,3-b]pyrazine,
2-Methoxy-7-(phenylethynyl)pyrido[2,3-b]pyrazine,
3-(p-Tolylethynyl)-[1,5]naphthyridine,
3-(o-Tolylethynyl)-[1,5]naphthyridine,
3-(m-Tolylethynyl)-[1,5]naphthyridine,
3-(2,4-Difluoro-phenylethynyl)-[1,5]naphthyridine,
3-(3,5-Difluoro-phenylethynyl)-[1,5]naphthyridine,
3-((4-(Trifluoromethyl)phenyl)ethynyl)-1,5-naphthyridine,
3-((3-(Trifluoromethyl)phenyl)ethynyl)-1,5-naphthyridine,
3-(Pyridin-4-ylethynyl)-1,5-naphthyridine,
3-(Pyridin-3-ylethynyl)-1,5-naphthyridine,
5-((1,5-Naphthyridin-3-yl)ethynyl)-N-methylpyridin-2-amine,
5-((1,5-Naphthyridin-3-yl)ethynyl)-N-methylpyrimidin-2-amine,
3-Methyl-6-phenylethynyl-3H-imidazo[4,5-b]pyridine,
6-(3-Fluoro-phenylethynyl)-3-methyl-3H-imidazo[4,5-b]pyridine,
6-(4-Fluoro-phenylethynyl)-3-methyl-3H-imidazo[4,5-b]pyridine,

and

optical isomers, prodrugs, pharmaceutically acceptable salts, hydrates, solvates, and polymorphs thereof.

Moreover, the invention relates to a compound of Formula I as defined above or an optical isomer, pharmaceutically acceptable salt, hydrate, solvate or polymorph thereof for the treatment and/or prevention of a condition or disease associated with abnormal glutamate neurotransmission, including a condition or disease which is affected or facilitated by modulation of the mGluR5 receptor, including for the conditions or diseases selected from those described earlier in the description.

A further aspect of the invention relates to a compound of Formula I as defined above or an optical isomer, pharmaceutically acceptable salt, hydrate, solvate or polymorph thereof for the treatment and/or prevention of a condition associated with abnormal glutamate neurotransmission or in which modulation of mGluR5 receptors results in therapeutic benefit and/or in the treatment or prevention of conditions in which MAO-B plays a role. The conditions which may be treated have already been described above. Such conditions and indications include:
a) For mGluR5 modulators: chronic pain, neuropathic pain, diabetic neuropathic pain (DNP), cancer pain, pain related to rheumathic arthritis, inflammatory pain, L-dopa-induced dyskinesias, dopaminomimetic-induced dyskinesias, L-dopa-induced dyskinesias in Parkinson's disease therapy, dopaminomimetic-induced dyskinesias in Parkinson's disease therapy, tardive dyskinesias, Parkinson's disease, anxiety disorders, panic disorders, anxiety and panic disorders, social anxiety disorder (SAD), generalized anxiety disorder, substance-induced anxiety disorder, eating disorders, obesity, binge eating disorders, Huntington's chorea, epilepsy, Alzheimer's disease, positive and negative symptoms of schizophrenia, cognitive impairment, functional gastrointestinal disorders, gastroesophageal reflux disease (GERD), migraine, irritable bowel syndrome (IBS), or for cognitive enhancement and/or neuroprotection.

b) Negative modulation of mGluR5 may be particularly useful for: chronic pain, neuropathic pain, diabetic neuropathic pain (DNP), cancer pain, pain related to rheumathic arthritis, inflammatory pain, L-dopa-induced dyskinesias, dopaminomimetic-induced dyskinesias, L-dopa-induced dyskinesias in Parkinson's disease therapy, dopaminomimetic-induced dyskinesias in Parkinson's disease therapy, tardive dyskinesias, Parkinson's disease, anxiety disorders, panic disorders, anxiety and panic disorders, social anxiety disorder (SAD), generalized anxiety disorder, substance-induced anxiety disorder, eating disorders, obesity, binge eating disorders, migraine, irritable bowel syndrome (IBS), functional gastrointestinal disorders, gastroesophageal reflux disease (GERD), Huntington's chorea and/or epilepsy.
c) Positive modulation of mGluR5 may be particularly useful for: Alzheimer's disease, positive and/or negative symptoms of schizophrenia, cognitive impairment, or for cognitive enhancement and/or neuroprotection.
d) Inhibition of MAO-B may be particularly useful for neurodegenerative diseases including Alzheimer's disease and Parkinson's disease. Inhibition of MAO-B may also be useful for smoking cessation, depression and/or mood stabilization.

A further aspect of the invention relates to a compound of Formula I as defined above or an optical isomer, pharmaceutically acceptable salt, hydrate, solvate or polymorph thereof for the treatment of binge eating disorders.

Further, the invention relates to the use of a compound of Formula I as defined above or an optical isomer, pharmaceutically acceptable salt, hydrate, solvate or polymorph thereof for the preparation of a medicament for treating or preventing a condition or disease associated with abnormal glutamate neurotransmission. Such a use includes the use of such a compound for the preparation of a medicament for the prevention and/or treatment of a condition or disease in an animal including a human being which condition or disease is affected or facilitated by modulation of the mGluR5 receptor.

Moreover, the invention relates to a method for treating or preventing a condition associated or disease associated with abnormal glutamate neurotransmission, including a condition or disease which is affected or facilitated by modulation of the mGluR5 receptor, including for the conditions or diseases selected from those described earlier in the description.

In a further embodiment, the invention relates to a compound of Formula I as described herein for use in the treatment or prevention of abnormal glutamate neurotransmission. This use may be in the treatment or prevention of a condition or disease as described herein.

Further, the invention relates to a pharmaceutical composition comprising as active ingredient at least one compound of Formula I as defined above or an optical isomer, pharmaceutically acceptable salt, hydrate, solvate or polymorph thereof, together with one or more pharmaceutically acceptable excipients.

Moreover, the mGluR modulators as described above are expected to have a high activity when administered in combination with other substances exhibiting neurological effects via different mechanisms.

A further aspect of the invention relates to a pharmaceutical composition comprising at least two different active ingredients, selected from least one compound of Formula I as defined above, and, additionally, at least one NMDA-antagonist, together with one or more pharmaceutically acceptable excipients. These compositions may be used for the treatment of CNS-related diseases, cognitive enhancement and for neuro-protection. The invention thus additionally provides a composition comprising at least two different active ingredients, selected from least one compound of Formula I as defined above, and, additionally, at least one NMDA-antagonist for the treatment of any of the conditions indicated herein, including CNS-related diseases, cognitive enhancement and for neuro-protection.

This invention also relates to a pharmaceutical composition comprising a combination of a compound of Formula I as described above and an NMDA receptor antagonist, including compositions wherein the NMDA receptor antagonist is selected from Memantine and Neramexane (or a combination thereof) and pharmaceutically acceptable salts, polymorphs, hydrates and solvates thereof.

The invention also relates to a pharmaceutical composition comprising at least two different active ingredients, selected from at least one compound of Formula I as defined above, and, additionally, at least one active ingredient selected from L-DOPA, other dopaminomimetics (such as antiparkinsonian dopaminomimetics, including bromocriptine, cabergolin, ropinirole, pramiperole, pergolide, rotigotine), and neuroleptics (such as classical neuroleptics, including haloperidol, perphenazin, chlorpromazine, metoclopramide).

The invention also relates to a method of providing neuroprotection in a living animal, including a human, comprising the step of administering to a living animal, including a human, a therapeutically effective amount of a composition as described above.

In a further embodiment, the invention relates to a compound of Formula I as described herein for use in providing neuroprotection. This use may be in a method for providing neuroprotection as described herein.

Furthermore, the invention relates to the use of a composition as described above for the manufacture of a medicament to provide neuroprotection in an animal, including a human.

This invention also relates to a method for treating or preventing a condition or disease in which MAO-B plays a role, including for the conditions or diseases selected from those described earlier in the description.

Furthermore, the invention relates to the a compound of Formula I as described herein for use in inhibiting MAO-B. This use may be in the treatment or prevention of a condition or disease as described herein.

The invention also relates to a process for the synthesis or preparation of a compound of Formula IA

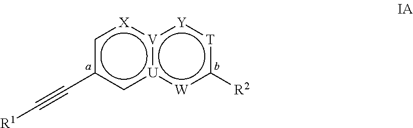

wherein R¹, R², T, U, V, X, Y, and W are as defined above for Formula I, wherein a compound of Formula II

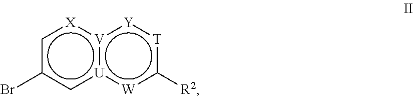

is treated with an arylacetylene of Formula III

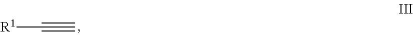

in the presence of a suitable catalyst, such as PdCl₂(PPh₃)₂, to yield a compound of Formula IA, which may be converted to a prodrug, pharmaceutically acceptable salt, hydrate, solvate, or polymorph.

The invention also relates to a process for the synthesis or preparation of a compound of Formula IA

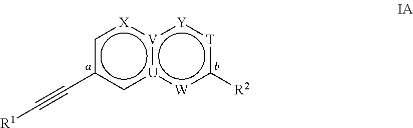

wherein R¹, R², T, U, V, X, Y, and W are as defined above for Formula I, wherein a compound of Formula II

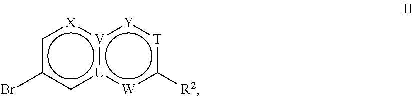

is treated with trimethylacetylene to yield, after removal of the TMS group under appropriate conditions, a compound of Formula IV

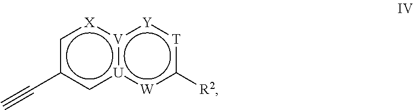

which is reacted with a compound of Formula V

R¹-Hal V,

in the presence of a suitable catalyst, such as, PdCl₂(PPh₃)₂, to yield a compound of Formula IA, which may be converted to a prodrug, pharmaceutically acceptable salt, hydrate, solvate, or polymorph.

The invention also relates to a process for the synthesis or preparation of a compound of Formula IB

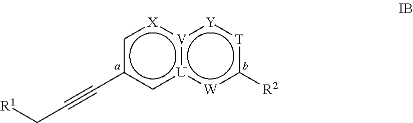

wherein R¹, R², T, U, V, X, Y, and W are as defined above for Formula I, wherein a compound of Formula II

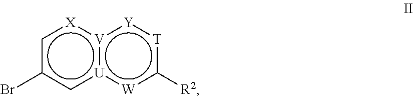

is treated with trimethylacetylene to yield, after removal of the TMS group under appropriate conditions, a compound of Formula IV

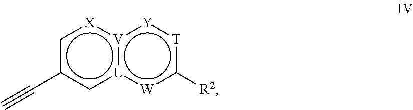

which is reacted with a compound of Formula VI

R¹—CH₂ZnBr VI,

to yield a compound of Formula IB, which may be converted to a prodrug, pharmaceutically acceptable salt, hydrate, solvate, or polymorph.

### DETAILED DESCRIPTION OF THE INVENTION

For the purpose of the present invention, in the compounds of Formula I the carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, (C₁₋₃)alkyl refers to alkyl of one to three carbon atoms (i.e. 1, 2 or 3 carbon atoms), inclusive, (i.e., methyl, ethyl, propyl, and isopropyl), straight and branched forms thereof, (C₁₋₆) for instance refers to a radical of one to six carbon atoms (i.e. 1, 2, 3, 4, 5 or 6 carbon atoms).

As used herein, the following definitions are applicable unless otherwise described, the term “C₁₋₆alkyl” represents straight or branched chain alkyl groups which may be optionally substituted by one or more (e.g., 1, 2, 3, 4, or 5) substituents selected from halogen, trifluoromethyl, C₁₋₆alkoxy, amino, hydroxy, C₁₋₆alkylamino, and di-(C₁₋₆alkyl)amino. Examples of such alkyl groups include methyl, ethyl, n-propyl, 2-propyl, n-butyl, tert-butyl, —CF₃, —C₂F₆, —CBr₃ and —CCl₃.

The term “C₁₋₆alkylene” refers to a divalent “C₁₋₆alkyl” radical as defined above. Examples of such alkylene groups include methylene, ethylene, propylene, butylene, which groups may be straight or branched.

The term “C₂₋₆alkenyl” represents straight or branched chain alkenyl groups.

The term “C₁₋₆alkoxy” represents straight or branched chain —O—C₁₋₆alkyl groups which may be optionally substituted by one or more (e.g., 1, 2, 3, 4, or 5) substituents selected from halogen, trifluoromethyl, amino, hydroxy, C₁₋₆alkylamino and di-(C₁₋₆ alkyl)amino. Examples of such alkoxy groups include methoxy, ethoxy, n-propoxy, i-propoxy, —OCF₃ and —OC₂F₆.

The term “cycloC₃₋₁₂alkyl” represents monocyclic or bicyclic, or tricyclic alkyl groups, including cyclopropyl, cyclobutyl, cyclopentyl, cyclohexyl, bicyclo[2.2.1]heptyl and adamantanyl, which may be optionally substituted by one or more (e.g., 1, 2, 3, 4, or 5) substituents, which may be the same or different, selected independently from halogen, trifluoromethyl, trifluoromethoxy, C₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylamino, and di-(C₁₋₆ alkyl)amino, C₁₋₆alkylcarbonylamino, and C₁₋₆alkylenedioxy.

The term “cycloC₃₋₁₂alkenyl” represents monocyclic or bicyclic, or tricyclic alkenyl groups, including cyclopentenyl and cyclohexenyl, which may be optionally substituted by one or more (e.g., 1, 2, 3, 4, or 5) substituents, which may be the same or different, selected independently from halogen, trifluoromethyl, trifluoromethoxy, C₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylamino, and di-(C₁₋₆alkyl)amino, C₁₋₆alkylcarbonylamino, and C₁₋₆alkylenedioxy.

The term “heterocyclyl” represents a saturated or unsaturated 4-7 membered heterocycle containing one or two heteroatoms selected from oxygen, sulfur and nitrogen, which may be optionally substituted by one or more (e.g., 1, 2, 3, 4, or 5) substituents, which may be the same or different, selected independently from halogen, trifluoromethyl, trifluoromethoxy, C₁₋₆alkyl, C₂₋₆alkenyl, C₁₋₆alkoxy, amino, hydroxy, nitro, cyano, cyanomethyl, C₁₋₆alkoxycarbonyl, C₁₋₆alkylamino, and di-(C₁₋₆alkyl)amino, C₁₋₆alkylcarbonylamino, and C₁₋₆alkylenedioxy, examples of such heterocyclyl groups include azetidinyl, pyrrolidinyl, piperidinyl, azepanyl, tetrahydrofuryl, thiazolidinyl, morpholinyl, thiomorpholinyl, piperazinyl, dihydropyridyl, and dihydrothiopyranyl.

The term “aryl” represents phenyl or naphthyl, wherein the phenyl or naphthyl group is optionally substituted by one or more (e.g., 1, 2, 3, 4, or 5) substituents, which may be the same or different, selected independently from halogen, trifluoromethyl, trifluoromethoxy, C₁₋₆alkyl, hydroxyC₁₋₆alkyl, C₂₋₆alkenyl, C₁₋₆alkoxy, C₁₋₆alkoxyC₁₋₆alkyl, amino, hydroxy, nitro, cyano, formyl, cyanomethyl, C₁₋₆alkoxycarbonyl, C₁₋₆alkylcarbonyloxy, C₁₋₆alkylcarbonyloxyC₁₋₆alkyl, C₁₋₆alkylamino, di-(C₁₋₆alkyl)amino, C₁₋₆alkylcarbonylamino, phenylcarbonylamino, aminocarbonyl, N—C₁₋₆alkylaminocarbonyl, di-N,N—C₁₋₆alkylaminocarbonyl, pyrrolidinyl, piperidinyl, morpholinyl, piperazinyl, cycloC₃₋₁₂alkyl and C₁₋₆alkylenedioxy.

The term “heteroaryl” represents an aromatic 5-6 membered ring containing from one to four heteroatoms selected from oxygen, sulfur and nitrogen, or a bicyclic group comprising a 5-6 membered ring containing from one to four heteroatoms selected from oxygen, sulfur and nitrogen fused with a benzene ring or a 5-6 membered ring containing from one to four heteroatoms selected from oxygen, sulfur and nitrogen, wherein the heteroaryl group may be optionally substituted by one or more (e.g., 1, 2, 3, 4, or 5) substituents, which may be the same or different, selected independently from halogen, trifluoromethyl, trifluoromethoxy, C₁₋₆alkyl, hydroxyC₁₋₆alkyl, C₂₋₆alkenyl, C₁₋₆alkoxy, amino, hydroxy, nitro, cyano, C₁₋₆alkoxycarbonyl, C₁₋₆alkoxycarbonyloxy, C₁₋₆alkylamino, di-(C₁₋₆alkyl)amino, C₁₋₆alkylcarbonylamino, aminocarbonyl, N—C₁₋₆alkylaminocarbonyl, di-N,N—C₁₋₆alkylaminocarbonyl, pyrrolidinyl, piperidinyl, morpholinyl, cycloC₃₋₁₂alkyl, C₁₋₆alkylenedioxy and aryl. Representative heteroaryl groups include furyl, thienyl, pyrrolyl, oxazolyl, isoxazolyl, isothiazolyl, oxadiazolyl, pyrazolyl, triazolyl, thiadiazolyl, thiazolyl, imidazolyl, oxadiazolyl, tetrazolyl, pyridinyl, pyrimidinyl, pyridazinyl, pyrazinyl, triazinyl, purinyl, pyrazolyl, benzofuryl, benzothienyl, indolyl, indolizinyl, isoindolyl, indolinyl, indazolyl, benzimidazolyl, benzoxazolyl, benzothiazolyl, quinolinyl, quinazolinyl, quinoxalinyl, cinnolinyl, naphthyridinyl, isoquinolinyl, quinolizinyl, phthalazinyl, theridinyl.

The term “halogen” represents fluorine, chlorine, bromine and iodine.

The compounds of the present invention are usually named according to the IUPAC or CAS nomenclature system. Abbreviations which are well known to one of ordinary skill in the art may be used (e.g. “Ph” for phenyl, “Me” for methyl, “Et” for ethyl, “h” for hour or hours, and “rt” for room temperature).

Memantine, also known as 1-amino-3,5-dimethyladamantane, is disclosed, U.S. Pat. Nos. 4,122,193; 4,273,774; and 5,061,703, the subject matter of which patents is hereby incorporated by reference.

Neramexane, also known as 1-amino-1,3,3,5,5-pentamethylcyclohexane, is disclosed in detail in U.S. Pat. Nos. 6,034,134 and 6,071,966, the subject matter of which patents is hereby incorporated by reference.

Memantine and neramexane are systemically-active noncompetitive NMDA receptor antagonists having moderate affinity for the receptor. They exhibit strong voltage dependent characteristics and fast blocking/unblocking kinetics (see e.g. Görtelmeyer et al., Arzneim-Forsch/Drug Res., 1992, 42:904-913; Winblad et al., Int. J. Geriat. Psychiatry, 1999, 14:135-146; Rogawski, Amino Acids, 2000, 19: 133-49; Danysz et al., Curr. Pharm. Des., 2002, 8:835-43; Jirgensons et. al. Eur. J. Med. Chem., 2000, 35: 555-565).

The term “analog” or “derivative” is used herein in the conventional pharmaceutical sense, to refer to a molecule that structurally resembles a reference molecule, but has been modified in a targeted and controlled manner to replace one or more specific substituents of the reference molecule with an alternate substituent, thereby generating a molecule which is structurally similar to the reference molecule. Synthesis and screening of analogs (e.g., using structural and/or biochemical analysis), to identify slightly modified versions of a known compound which may have improved or biased traits (such as higher potency and/or selectivity at a specific targeted receptor type, greater ability to penetrate blood-brain barriers, fewer side effects, etc.) is a drug design approach that is well known in pharmaceutical chemistry.

In addition, using methods known to those skilled in the art, analogs and derivatives of the compounds of the invention may be created which have improved therapeutic efficacy, i.e., higher potency and/or selectivity at a specific targeted receptor type, either greater or lower ability to penetrate mammalian blood-brain barriers (e.g., either higher or lower blood-brain barrier permeation rate), fewer side effects, etc.

The term “prodrug” is used herein in the conventional pharmaceutical sense, to refer to a molecule which undergoes a transformation in vivo (e.g., an enzymatic or chemical transformation) to release an active parent drug. Prodrugs of the compounds of Formula I of the present invention may be prepared by chemically modifying a functional group present in the compound of Formula I such that the chemically modified compound may undergo a transformation in vivo (e.g., enzymatic hydrolysis) to provide the compound of Formula I. Examples of functional groups present in the compounds of Formula I which may be modified to produce prodrugs include carboxy, hydroxy, amino, and thio groups. Prodrugs of the compounds of Formula I of the present invention may be prepared according to conventional techniques which have been described in the art (see, for example, Stella V., et al., Prodrugs: Challenges and Rewards, AAPS Press/Springer, New York, 2007).

The phrase “pharmaceutically acceptable”, as used in connection with compositions of the invention, refers to molecular entities and other ingredients of such compositions that are physiologically tolerable and do not typically produce untoward reactions when administered to a mammal (e.g., human). The term “pharmaceutically acceptable” may also mean approved by a regulatory agency of the Federal or a state government or listed in the U.S. Pharmacopeia or other generally recognized pharmacopeia for use in mammals, and more particularly in humans.

Compounds of the present invention may be in the form of pharmaceutically acceptable salts. “Pharmaceutically acceptable salts” refers to those salts which possess the biological effectiveness and properties of the parent compound and which are not biologically or otherwise undesirable. The nature of the salt is not critical,

provided that it is non-toxic and does not substantially interfere with the desired pharmacological activity.

It will be appreciated by those skilled in the art that compounds of the invention having a chiral center may exist in and be isolated in optically active and racemic forms. Some compounds may exhibit polymorphism. It is to be understood that the present invention encompasses any racemic, optically active, polymorphic, tautomeric, or stereoisomeric form, or mixture thereof, of a compound of the invention, which possesses the useful properties described herein.

The following schemes describe the preparation of compounds of the present invention. Scheme 1 describes the preparation of compounds of Formula I of the present invention, and Schemes 2-9 describe methods for preparing starting materials and intermediates for use in the preparation of compounds of Formula I. All of the starting materials may be prepared by procedures described in these schemes, by procedures well known to one of ordinary skill in organic chemistry, or may be obtained commercially. All of the final compounds of the present invention may be prepared by procedures described in these charts or by procedures analogous thereto, which would be well known to one of ordinary skill in organic chemistry. All of the variables used in Schemes 1-9 are as defined below or as in the claims. Compounds containing one or more chiral centers may be prepared as racemates or mixtures of various stereoisomers and then separated. However, they also may be prepared by a special enantioselective synthesis. For several of the chiral compounds, the enantiomers differ in pharmacological activity.

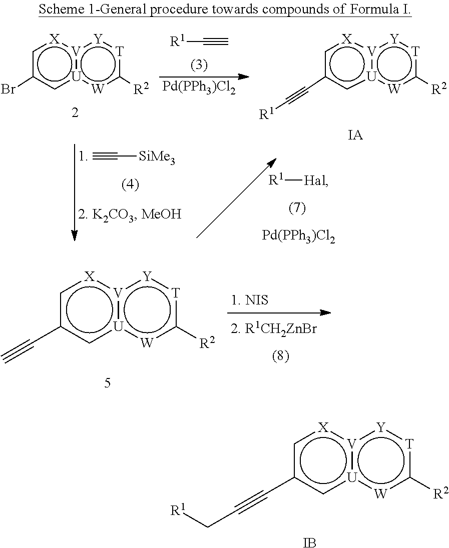

Sonogashira coupling of a bromo-heterocyclic compound 2 with an arylacetylene 3 in the presence of a suitable catalyst, such as Pd(PPh₃)₂Cl₂, provides an arylethynyl substituted derivative of Formula IA. Alternatively, a bromo-heterocyclic compound 2 may be reacted with trimethylsilylacetylene (4) to give, after cleavage of the TMS group, ethynyl-substituted compound 5. Compound 5 is converted to an arylethynyl substituted derivative of Formula IA via Sonogashira with an aryl halide 7. Compound 5 may also be first iodinated and then reacted with a benzylic organozinc reagent 8 to yield an arylpropargyl substituted derivative of Formula IB.

Methods for preparing bromo-heterocyclic compounds (2) are shown in Schemes 2-9.

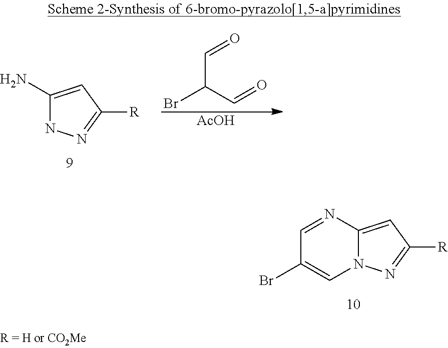

6-bromo-pyrazolo[1,5-a]pyrimidines (10) are prepared by condensation of commercially available 2H-pyrazol-3-ylamines 9 with bromomalonaldehyde as shown in Scheme 2. If 5-amino-1H-pyrazole-3-carboxylic acid methyl ester (R═CO₂Me) is used, the ester group in compound 10 may be hydrolyzed to an acid and subsequently converted to different amides using standard procedures.

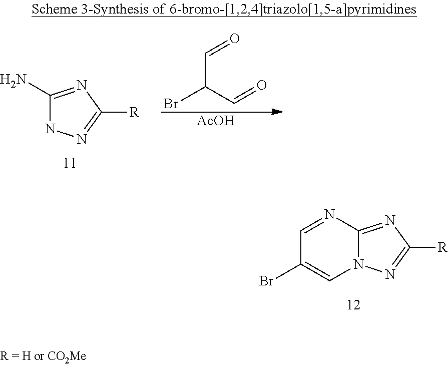

6-Bromo-[1,2,4]triazolo[1,5-a]pyrimidines 12 are prepared by condensation of commercially available 2H-[1,2,4]triazol-3-ylamines 11 with bromomalonaldehyde as shown in Scheme 3. If 5-amino-1H-[1,2,4]triazole-3-carboxylic acid methyl ester (R═CO₂Me) is used, the ester group in compound 12 can be hydrolyzed to an acid and subsequently converted to different amides using standard procedures.

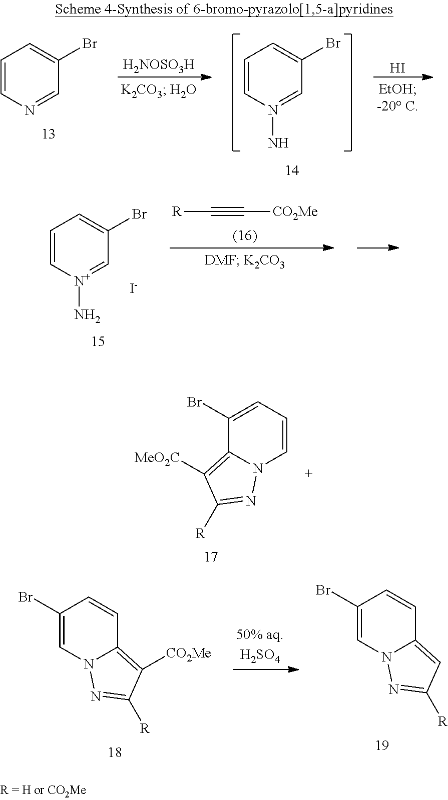

6-Bromo-pyrazolo[1,5-a]pyridines 19 are synthesized as shown in Scheme 4. Treatment of 3-bromopyridine (13) with hydroxylamine-O-sulfonic acid in the presence of potassium carbonate results in formation of the intermediate N-iminopyridinium species (14), which upon addition of hydroiodic acid cleanly affords 1-amino-pyridinium iodide (15). 1,3-dipolar cycloadditions of this aminopyridinium salt with dimethyl acetylenedicarboxylate or methyl propiolate proceed without regioselectivity to provide ca. 1:1 mixture of pyrazolo[1,5-a]pyridine-3-carboxylic acid methyl esters (17 and 18). These isomeric compounds are easily separable by flash column chromatography. Heating of the separated desired isomers in aqueous sulfuric acid results in ester hydrolysis with concomitant decarboxylation to give 6-bromopyrazolo[1,5-a]pyridine (R═H) or 6-bromopyrazolo[1,5-a]pyridine-2-carboxylic acid (R═CO₂H) (19). The carboxylic acid derivatives may be converted to a variety of derivatives, such as esters and amides using conventional methods.

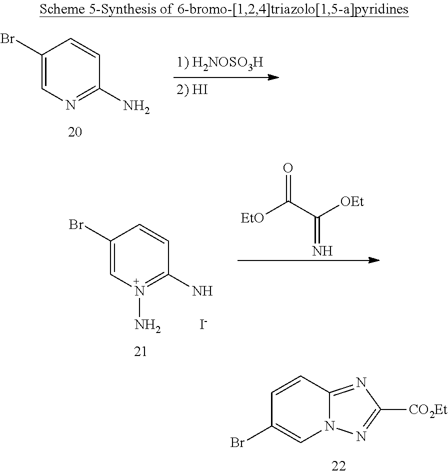

Unsubstituted 6-bromo-[1,2,4]triazolo[1,5-a]pyridine is commercially available (Combi-Blocks, Apollo Scientific). 6-Bromo-[1,2,4]triazolo[1,5-a]pyridine-2-carboxylic acid ethyl ester 22 (Scheme V) is synthesized from 2-amino-6-bromopyridine 20 according to a published procedure (Gómez, E., Avendaño, C., McKillop, A. Tetrahedron, 1986, 42 (10), 2625-2634). The ester 22 can be hydrolyzed to an acid and subsequently converted to a variety of amides using standard procedures.

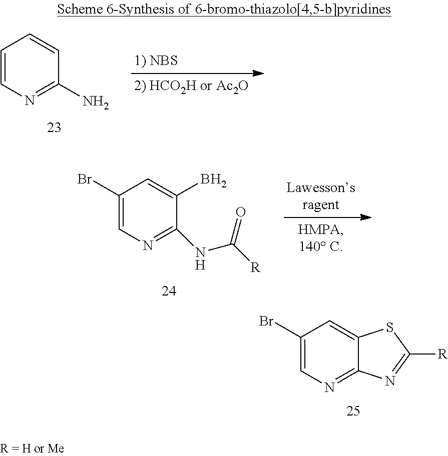

6-Bromo-thiazolo[4,5-b]pyridines 25 are synthesized as shown in Scheme VI. Bromination of 2-aminopyridine (23) with NBS, followed by formylation or acetylation of the resultant 2-amino-3,6-dibromopyridine provides formyl (R═H) or acetyl (R=Me) intermediate 24, treatment of which with Lawesson's reagent in HMPA at elevated temperature yields the corresponding 6-bromo-thiazolo[4,5-b]pyridine 25. The methyl group in compound 25 may be oxidized to a carboxylic acid (KMnO₄ or 1. SeO₂, 2. NaClO₂), which subsequently may be converted to a variety of amides using standard procedures.

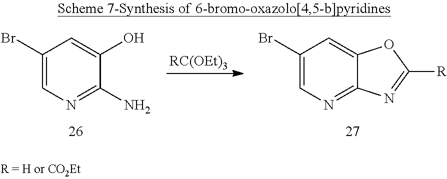

6-Bromo-oxazolo[4,5-b]pyridines 27 are prepared from 2-amino-5-bromo-3-hydroxypyridine (26), which is synthesized by a known method (Guillaumet, G. et. al. Heterocycles, 1995, 41 (12) 2799-2809), as shown in Scheme 7. Treatment of compound 26 with ethyl orthoformate yields unsubstituted 6-bromo-oxazolo[4,5-b]pyridine 27, whereas reaction of compound 26 with triethoxy-acetic acid ethyl ester provides ester 27, which may be hydrolized to an acid and converted to different amides using known methods.

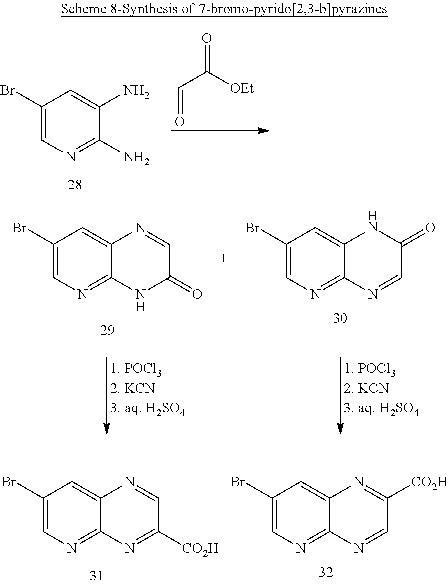

Unsubstituted 7-bromo-pyrido[2,3-b]pyrazine is commercially available (Apollo Scientific, Boron Molecular). 7-Bromo-pyrido[2,3-b]pyrazine-2-carboxylic acid 32 and 7-Bromo-pyrido[2,3-b]pyrazine-3-carboxylic acid 31 are synthesized as shown in Scheme 8. Condensation of 2,3-diamino-5-bromopyridine 28 with ethyl glyoxalate yields a mixture of lactams 29 and 30. The mixture is treated with POCl₃ to give the corresponding chloro derivatives, which are separated. Substitution of chlorine with a cyano group in each of the isomers, followed by acidic hydrolysis of the cyano group provides carboxylic acids 31 and 32 which may be converted to a variety of amides using standard procedures.

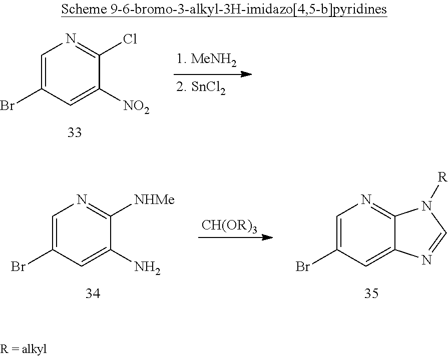

6-bromo-3-alkyl-3H-imidazo[4,5-b]pyridines 35 are synthesized as shown in Scheme 9. Reaction of 5-bromo-2-chloro-3-nitro-pyridine 33 with methylamine, followed by nitro group reduction with tin (II) chloride, provides an intermediate 5-bromo-N*2*-alkyl-pyridine-2,3-diamine 34. Treatment of compound 34 with trialkylorthoformate (e.g., trimethylorthoformate) at elevated temperature provides compound 35.

It will be appreciated that in the above transformations it may be necessary or desirable to protect any sensitive groups in the molecule of the compound in question in order to avoid undesirable side reactions.

Pure stereoisomeric forms (including optical isomers) of the compounds and the intermediates of this invention may be obtained by the application of art-known procedures. Diastereomers may be separated by physical separation methods such as selective crystallization and chromatographic techniques, e.g. liquid chromatography using chiral stationary phases. Enantiomers (optically active isomers) may be separated from each other by selective crystallization of their diastereomeric salts with optically active acids. Alternatively, enantiomers may be separated by chromatographic techniques using chiral stationary phases.

Pure stereoisomeric forms may also be derived from the corresponding pure stereoisomeric form of appropriate starting materials, provided that the reaction occur stereoselectively. Stereoisomeric forms of Formula I are included within the scope of this invention.

Compounds of Formula I which are marked by radioactive atoms may be obtained using art-known procedures. Typical compounds include those where one or more hydrogens are substituted by tritium, where one or more C¹² are substituted by C¹⁴, where one or more fluor atoms are substituted by F¹⁸ or other isotopes. These may be used for the treatment of diseases (e.g. cancer) but also for diagnostic purposes. The radioactive atoms exchanged in the molecule are often isotopes of carbon, hydrogen, halogen, sulphur or phosphorus. Compounds of the Formula I which are marked by radioactive atoms are included within the scope of this invention.

### Addition Salts

For therapeutic use, salts of the compounds of Formula I are those wherein the counterion is pharmaceutically acceptable. However, salts of acids and bases, which are non-pharmaceutically acceptable, may also find use, for example, in the preparation and purification of pharmaceutically acceptable compounds. All salts whether pharmaceutically acceptable or not are included within the ambit of the present invention. The pharmaceutically acceptable salts as mentioned above are meant to comprise the therapeutically active non-toxic salt forms, which the compounds of Formula I are able to form. The latter may conveniently be obtained by treating the base form with such appropriate acids as inorganic acids, e.g. hydrohalic acids such as hydrochloric, hydrobromic and the like; sulfuric acid; nitric acid; phosphoric acid and the like; or organic acids such as acetic, propanoic, hydroxyacetic, 2-hydroxypropanoic, oxopropanoic, oxalic, malonic, succinic, maleic, fumaric, malic, tartaric, 2-hydroxy-1,2,3-propanetricarboxylic, methanesulfonic, ethanesulfonic, benzenesulfonic, 4-methylbenzenesulfonic, cyclohexanesulfonic, 2-hydroxybenzoic, 4-amino-2-hydroxybenzoic and the like acids. Conversely, the salt form may be converted by treatment with alkali into the free base form.

### Pharmaceutical Compositions

The active ingredients of the compounds of the invention, together with one or more excipients such as adjuvants, carriers, or diluents, may be placed into the form of pharmaceutical compositions, unit dosages or dosage forms. The pharmaceutical compositions may be employed as solid dosage forms, such as powders, granules, pellets, coated or uncoated tablets or filled capsules, or liquid dosage forms, such as solutions, suspensions, emulsions, or capsules filled with the same, or semi solid dosage forms, such as gels, creams and ointments. The active ingredient(s) dissolution and release profiles of the pharmaceutical dosage forms may be varied from seconds to months.

The pharmaceutical compositions are designed for the use in animals and humans and may be applied via all application routes. Preferred application routes will be the oral route, the dermal route, the pulmonary route, the nasal route, the rectal route, the parenteral route. Such pharmaceutical compositions and unit dosage forms thereof may comprise conventional or new ingredients in conventional or special proportions, with or without additional active compounds or principles, and such unit dosage forms may contain any suitable effective amount of the active ingredient commensurate with the intended daily dosage range to be employed. Tablets containing one (1) to one hundred (100) milligrams of active ingredient or, more broadly, zero point five (0.5) to five hundred (500) milligrams per tablet, are accordingly suitable representative unit dosage forms.

The term “carrier” applied to pharmaceutical compositions of the invention refers to a diluent, excipient, or vehicle with which an active compound is administered. Such pharmaceutical carriers may be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. A. R. Gennaro, 20^{th} Edition, describes suitable pharmaceutical carriers in “Remington: The Science and Practice of Pharmacy”.

### Method of Treating

Due to their high degree of activity and their low toxicity, together presenting a most favorable therapeutic index, the active principles of the invention may be administered to a subject, e.g., a living animal (including a human) body, in need thereof, for the treatment, alleviation, or amelioration, palliation, or elimination of an indication or condition which is susceptible thereto, or representatively of an indication or condition set forth elsewhere in this application, preferably concurrently, simultaneously, or together with one or more pharmaceutically-acceptable excipients, carriers, or diluents, especially and preferably in the form of a pharmaceutical composition thereof, whether by oral, rectal, or parental (including intravenous and subcutaneous) or in some cases even topical route, in an effective amount. Suitable dosage ranges are 1-1000 milligrams daily, optionally 10-500 milligrams daily, and optionally 50-500 milligrams daily, depending as usual upon the exact mode of administration, form in which administered, the indication toward which the administration is directed, the subject involved and the body weight of the subject involved, and the preference and experience of the physician or veterinarian in charge.

The term “treat” is used herein to mean to relieve or alleviate at least one symptom of a disease in a subject. Within the meaning of the present invention, the term “treat” also denotes to arrest, delay the onset (i.e., the period prior to clinical manifestation of a disease) and/or reduce the risk of developing or worsening a disease.

The term “combination” is used herein to define a single pharmaceutical composition (formulation) comprising a compound of the present invention and a second active ingredient (e.g., an NMDA receptor antagonist, L-DOPA, a dopaminomimetic, or a neuroleptic), in a formulation known in the art, or two separate pharmaceutical compositions (formulations), one comprising a compound of the present invention as formulated above and one comprising a second active ingredient (e.g., an NMDA receptor antagonist, L-DOPA, a dopaminomimetic, or a neuroleptic) in a formulation known in the art, to be administered conjointly.

Within the meaning of the present invention, the term “conjoint administration” is used to refer to administration of a compound of the present invention and a second active ingredient (e.g., an NMDA receptor antagonist, L-DOPA, a dopaminomimetic, or a neuroleptic) in one composition, or simultaneously in different compositions, or sequentially. For the sequential administration to be considered “conjoint”, however, the compound of the present invention and the NMDA receptor antagonist must be administered separated by a time interval that still permits the resultant beneficial effect in a mammal. For example, the compound of the present invention and the NMDA receptor antagonist must be administered on the same day (e.g., each—once or twice daily), including within an hour of each other, and including simultaneously.

The term “therapeutically effective” applied to dose or amount refers to that quantity of a compound or pharmaceutical composition that is sufficient to result in a desired activity upon administration to a living animal body in need thereof.

Compounds of the present invention may be administered orally, topically, parenterally, or mucosally (e.g., buccally, by inhalation, or rectally) in dosage unit formulations containing conventional non-toxic pharmaceutically acceptable carriers. It is usually desirable to use the oral route. The active agents may be administered orally in the form of a capsule, a tablet, or the like (see Remington: The Science and Practice of Pharmacy, 20^{th} Edition). The orally administered pharmaceutical compositions may be administered in the form of a time-controlled release vehicle, including diffusion-controlled systems, osmotic devices, dissolution-controlled matrices, and erodible/degradable matrices.

For oral administration in the form of a tablet or capsule, the active drug component of Formula I may be combined with non-toxic, pharmaceutically acceptable excipients such as binding agents (e.g., pregelatinized maize starch, polyvinylpyrrolidone or hydroxypropyl methylcellulose); fillers (e.g., lactose, sucrose, glucose, mannitol, sorbitol and other reducing and non-reducing sugars, microcrystalline cellulose, calcium sulfate, or calcium hydrogen phosphate); lubricants (e.g., magnesium stearate, talc, or silica, steric acid, sodium stearyl fumarate, glyceryl behenate, calcium stearate, and the like); disintegrants (e.g., potato starch or sodium starch glycolate); and/or wetting agents (e.g., sodium lauryl sulphate), coloring and flavoring agents, gelatin, sweeteners, natural and synthetic gums (such as acacia, tragacanth or alginates), buffer salts, carboxymethylcellulose, polyethyleneglycol, waxes, and the like. For oral administration in liquid form, the drug components may be combined with non-toxic, pharmaceutically acceptable inert carriers (e.g., ethanol, glycerol, water), suspending agents (e.g., sorbitol syrup, cellulose derivatives or hydrogenated edible fats), emulsifying agents (e.g., lecithin or acacia), non-aqueous vehicles (e.g., almond oil, oily esters, ethyl alcohol or fractionated vegetable oils), preservatives (e.g., methyl or propyl-p-hydroxybenzoates or sorbic acid), and the like. Stabilizing agents such as antioxidants (BHA, BHT, propyl gallate, sodium ascorbate, citric acid) may also be added to stabilize the dosage forms.

Tablets may be coated by methods well known in the art. Compositions of the invention containing as active compound a compound of Formula I may be also introduced in beads, microspheres or microcapsules, e.g., fabricated from polyglycolic acid/lactic acid (PGLA). Liquid preparations for oral administration may take the form of, for example, solutions, syrups, emulsions or suspensions, or they may be presented as a dry product for reconstitution with water or other suitable vehicle before use. Preparations for oral administration may be suitably formulated to give controlled or postponed release of the active compound.

Compounds of the present invention may also be administered in the form of liposome delivery systems, such as small unilamellar vesicles, large unilamellar vesicles and multilamellar vesicles. Liposomes may be formed from a variety of phospholipids, such as cholesterol, stearylamine or phosphatidylcholines, as is well known.

Compounds of the present invention may also be delivered by the use of monoclonal antibodies as individual carriers to which the compound molecules are coupled. Active drugs may also be coupled with soluble polymers as targetable drug carriers. Such polymers include polyvinyl-pyrrolidone, pyran copolymer, polyhydroxy-propyl methacrylamide-phenol, polyhydroxy-ethyl-aspartamide-phenol, or polyethyleneoxide-polylysine substituted with palmitoyl residues. Furthermore, active drug may be coupled to a class of biodegradable polymers useful in achieving controlled release of a drug, for example, polylactic acid, polyglycolic acid, copolymers of polylactic and polyglycolic acid, polyepsilon caprolactone, polyhydroxybutyric acid, polyorthoesters, polyacetals, polyhydropyrans, polycyanoacrylates, and cross-linked or amphipathic block copolymers of hydrogels.

For administration by inhalation, the therapeutics according to the present invention containing as active compound a compound of Formula I may be conveniently delivered in the form of an aerosol spray presentation from pressurized packs or a nebulizer, with the use of a suitable propellant, e.g., dichlorodifluoromethane, trichlorofluoromethane, dichlorotetra-fluoroethane, carbon dioxide, or other suitable gas. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Formulations comprising compounds of the present invention may be delivered parenterally, i.e., by intravenous (i.v.), intracerebroventricular (i.c.v.), subcutaneous (s.c.), intraperitoneal (i.p.), intramuscular (i.m.), subdermal (s.d.), or intradermal (i.d.) administration, by direct injection, via, for example, bolus injection or continuous infusion. Formulations for injection may be presented in unit dosage form, e.g., in ampoules or in multi-dose containers, with an added preservative. The compositions may take such forms as excipients, suspensions, solutions, or emulsions in oily or aqueous vehicles, and may contain formulatory agents such as suspending, stabilizing and/or dispersing agents. Alternatively, the active ingredient may be in powder form for reconstitution with a suitable vehicle, e.g., sterile pyrogen-free water, before use.

Compounds of the present invention may also be formulated for rectal administration, e.g., as suppositories or retention enemas (e.g., containing conventional suppository bases such as cocoa butter or other glycerides).

Compositions containing a compound of Formula I may, if desired, be presented in a pack or dispenser device, which may contain one or more unit dosage forms containing the active ingredient and/or may contain different dosage levels to facilitate dosage titration. The pack may, for example, comprise metal or plastic foil, such as a blister pack. The pack or dispenser device may be accompanied by instructions for administration. Compositions of the invention formulated in a compatible pharmaceutical carrier may also be prepared, placed in an appropriate container, and labeled for treatment of an indicated condition.

As disclosed herein, the dose of the components in the compositions of the present invention is determined to ensure that the dose administered continuously or intermittently will not exceed an amount determined after consideration of the results in test animals and the individual conditions of a patient. A specific dose naturally varies depending on the dosage procedure, the conditions of a patient or a subject animal such as age, body weight, sex, sensitivity, feed, dosage period, drugs used in combination, seriousness of the disease. The appropriate dose and dosage times under certain conditions may be determined by the test based on the above-described indices but may be refined and ultimately decided according to the judgment of the practitioner and each patient's circumstances (age, general condition, severity of symptoms, sex, etc.) according to standard clinical techniques.

Toxicity and therapeutic efficacy of the compositions of the invention may be determined by standard pharmaceutical procedures in experimental animals, e.g., by determining the LD₅₀ (the dose lethal to 50% of the population) and the ED₅₀ (the dose therapeutically effective in 50% of the population). The dose ratio between therapeutic and toxic effects is the therapeutic index and it may be expressed as the ratio LD₅₀/ED₅₀. Compositions that exhibit large therapeutic indices are preferred.

### Experimental Part

The compounds and their preparation of the present invention will be better understood in connection with the following examples, which are intended as an illustration of and not a limitation upon the scope of the invention.

Hereinafter, “DMF” is defined as N,N-dimethylformamide, “THF” as tetrahydrofurane, “HCl” as hydrochloric acid, “NaOH” as sodium hydroxide, “MeOH” as methanol, “DMSO” as dimethylsulfoxide and “TBTU” as O-(benzotriazol-1-yl)-N,N,N′,N′-tetramethyluronium tetrafluoroborate.

### General Procedure 1

### Sonogashira Coupling of Bromo-Heterocyclic Compounds with Substituted Acetylenes

Under a strong stream of argon PdCl₂(PPh₃)₂ (0.018 mmol, 0.018 g), CuI (0.018 mmol, 0.003 g) and 6-bromo-heterocyclic compound (0.36 mmol) are placed in a vial. A solution of substituted acetylene (0.20 mmol) in 2 mL DMF is then added. The mixture is flushed thoroughly with argon and Et₃N (0.7105 mmol, 0.0718 g, 0.097 mL) is added through the septum, the reaction mixture immediately turns clear. The mixture is stirred at 60-65° C. for 16 h. The reaction mixture is then partitioned between H₂O and EtOAc. The organic layer is washed with brine (3×25 mL), dried over Na₂SO₄, filtrated, evaporated and purified by flash column chromatography on silica gel (eluent hexane-EtOAc) to give the title compound.

### Preparation 1

### 6-Bromo-pyrazolo[1,5-a]pyrimidine

A solution of 3-amino-1H-pyrazole (120 mmol, 10 g) in 50 ml of anhydrous acetic acid is added dropwise to a suspension of bromomalonaldehyde (120 mmol, 18.9 g) in 50 mL anhydrous acetic acid at room temperature. The resulting brown solution is stirred at room temperature for 3 hours, acetic acid is evaporated at reduced pressure and chloroform is added to the residue. The organic phase is washed with saturated solution of NaHCO₃, brine, and dried over sodium sulfate. The product is purified by flash column chromatography (CH₂Cl₂: Et₃N 100:0.5 as eluent, R_{f}=0.3) to yield the title compound (5 g, 42%).

### Preparation 2

### 6-Bromo-pyrazolo[1,5-a]pyridine

To a freshly prepared solution of hydroxylamine-O-sulfonic acid (8 g, 70.7 mmol) in 50 mL of ice water 3-bromopyridine (6.9 mL, 70.7 mmol) is added. The mixture is heated at 90° C. for 30 min, then cooled to room temperature and K₂CO₃ (9.7 g, 70.7 mmol) is added. The water is evaporated (bath temperature 30-40° C.) and the crude product is treated with EtOH (100 mL). The precipitate is filtered and the filtrate is concentrated to small volume (20 mL) and HI (5.4 mL) is added. The reaction mixture is then evaporated to dryness in vacuo. The residue is dissolved in dry DMF (50 mL) and anhydrous K₂CO₃ (11.7 g, 84.8 mmol) is added. Methyl propiolate (7 ml, 84.8 mmol) is then added dropwise. The mixture is stirred in open flask for 24 h, then diluted with water (50 mL) and extracted with EtOAc (2×150 mL). The organic layers are combined, washed with brine (2×100 ml), dried over Na₂SO₄. The residue is purified by column chromatography (hexane: EtOAc 8:1) to give 6-bromo-pyrazolo[1,5-a]pyridine-3-carboxylic acid methyl ester and 4-bromo-pyrazolo[1,5-a]pyridine-3-carboxylic acid methyl ester in ca. 1:1 ratio.

6-Bromo-pyrazolo[1,5-a]pyridine-3-carboxylic acid methyl ester in 40% H₂SO₄ (˜60 mL) is heated at 110° C. for 3 h. The reaction mixture is cooled to 0° C., neutralized with saturated solution of NaHCO₃, and extracted with methylene chloride. The organic layers are combined, washed with brine and dried. The solvent is evaporated, and the residue is purified by column chromatography (hexane:ethyl acetate) and recrystallized from hexane to give the title compound.

### Preparation 3

### 6-Bromo-[1,2,4]triazolo[1,5-a]pyrimidine

A solution of 3-amino-1H-triazole (6 mmol, 0.5 g) in 5 mL of anhydrous acetic acid is added dropwise to a suspension of bromomalonaldehyde (6 mmol, 0.9 g) in 5 mL anhydrous acetic acid at room temperature. The resulting mixture is heated to 80° C. for 7 h, then acetic acid is evaporated at reduced pressure and DCM is added to the residue. The organic phase is washed with saturated solution of NaHCO₃, brine, dried over sodium sulfate and evaporated to dryness to give 918 mg (78%) of sufficiently pure title compound.

### Preparation 4

### 6-Bromo-thiazolo[4,5-b]pyridine

3,5-dibromo-2-N-formylaminopyridine (470 mg, 1.68 mmol) is dissolved in HMPA (3 ml), Lawesson's reagent (340 mg, 1.68 mmol) is added and the mixture is heated to 100° C. for 2 h. The mixture is cooled, diluted with DCM and washed with water. The organic phase is dried over anhydrous sodium sulfate and evaporated to dryness. Purification of the residue by flash column chromatography provides 50 mg of the title compound along with recovered starting material.

### Preparation 5

### 6-Bromo-oxazolo[4,5-b]pyridine

2-Amino-5-bromo-3-hydroxypyridine (200 mg, 1.06 mmol) is dissolved in 3 ml of triethylortoformate and a catalytic amount of p-toluenesulfonic acid is added. The mixture is heated to reflux for 5 h, then cooled, diluted with DCM and washed with saturated sodium bicarbonate solution. The organic phase is dried over anhydrous sodium sulfate and evaporated to dryness. The residue is purified by flash column chromatography to afford 212 mg (60%) of the title compound.

### Preparation 6

### 3-Bromo-[1,5]naphthyridine

Naphthyridine (200 mg, 1.53 mmol) is dissolved in 2 mL of acetic acid, sodium acetate (300 mg, 3.07 mmol) is added, the mixture is heated to 85° C. and a solution of bromine (0.087 mL, 270 mg, 1.69 mmol) in 0.3 mL acetic acid is added dropwise. The mixture is heated for 3 h, cooled and evaporated to dryness. The residue is purified by flash column chromatography to give 65 mg of title compound along with dibrominated product.

### General Procedure 2

### Coupling of Heterocyclic Carboxylic Acids with Amines

A solution of 0.12 mmol of heterocyclic carboxylic acid, 0.13 mmol of amine, 0.12 mmol of EDC and 0.12 mmol of HOBt in 2 mL of DMF is stirred at room temperature for 24 h. The mixture is concentrated in vacuo and the solid residue is partitioned between methylene chloride (3 ml) and saturated aqueous sodium hydrocarbonate solution (3 ml). The organic layer is separated, washed with water and brine and concentrated in vacuo. Purification of the residue by flash column chromatography (chloroform:methanol or hexane:EtOAc) gives the title compound.

### Preparation 7

### 6-Bromo-pyrazolo[1,5-a]pyrimidine-2-carboxylic acid

5-Nitro-3-pyrazole-carboxylic acid methyl ester (22.35 g, 130.61 mmol) is dissolved in 160 mL THF and 160 mL glacial acetic acid. Then, Pd—C (10%, 4.36 g) is added and the reaction is stirred for 6 days under hydrogen atmosphere at RT. Then, the mixture is filtered over celite and the solvent is removed under vacuum. The crude material is dissolved in methylene chloride (800 mL) and sodium hydrogen carbonate (200 g) is added, filtered and the solvent is again removed under vacuum. This procedure is repeated until the acetic acid smell is lost. 5-Amino-3-pyrazole-carboxylic acid methyl ester is isolated in high yields (16.91 g, 91.7%)

5-Amino-3-pyrazolcarboxylic acid methyl ester (16.91 g, 119.8 mmol) is dissolved in ethanol (2.4 L) and hydrochloric acid (37%, 12.5 mL, 150 mmol) is added. Then, a solution of 2-bromo-malonaldehyde (18.9 g, 125.2 mmol) in ethanol (1.4 L) and is quickly added in a dropwise manner at RT. After 30 min, a precipitation is observed; after 6 hours the precipitate is filtered and washed with 50 mL ethanol and thereafter with 50 mL diethyl ether to give 4.19 g of 6-bromo-pyrazolo[1,5a]pyrimidine-2-carboxylic acid methyl ester. After evaporation of the filtrate and crystallisation, an additional 1.43 g of product is obtained. Totally 5.62 g (18.3%).

6-Bromopyrazolo[1,5a]pyrimidin-2-carboxylic acid methyl ester (3.76 g, 14.68 mmol) is heated in 600 mL water, 190 mL sulfuric acid (30%) and 50 mL of the methanol/water mixture is removed from the reaction mixture via distillation. After cooling down, 50 mL water is added, the mixture is heated again and 50 mL of the alcohol-water mixture is removed. This cycle is repeated 6 times, the reaction mixture is cooled to RT and filtered over a glass filter. The crude material is washed with water (100 mL), acetone (20 mL) and ether (20 mL) and dried under vacuum to give 6-Bromo-pyrazolo[1,5a]pyrimidine-2-carboxylic acid (2.61 g, 73.5%).

### Preparation 8

### 6-Bromo-pyrazolo[1,5-a]pyridine-2-carboxylic acid

To a freshly prepared solution of 11.3 g (0.1 mol) of hydroxylamine-β-sulfonic acid in 60 mL of cold water is added 0.1 mol of 3-bromopyridine. The mixture is heated at 90° C. for 20 min, then cooled to room temperature and potassium carbonate (13.8 g, 0.1 mol) is added, followed by the removal of water by evaporation in vacuo. The residue is treated with 120 mL of ethanol and the insoluble precipitate of potassium sulfate is removed by filtration. The filtrate is treated with 14 mL of 57% hydroiodic acid and stored at −20° C. The solid that separates is collected by filtration to give 1-amino-3-bromopyridinium iodide which is used without further purification.

Dimethyl acetylenedicarboxylate (3.50 g, 24.6 mmol) is added dropwise to a stirred suspension of 1-amino-3-bromopyridinium iodide (23.5 mmol) and potassium carbonate (4.70 g, 47.5 mmol) in 40 mL of DMF at room temperature. The mixture is stirred for 2 h while a stream of air is introduced under the liquid level. After filtration and subsequent evaporation of the solvent in vacuo, the residue is treated with water (100 mL) and extracted with diethyl ether (3×100 mL). The organic layer is dried over anhydrous sodium sulfate, filtered and evaporated in vacuo. Purification of the residue by flash column chromatography (hexane-EtOAc 1:1) provides 6-bromo-pyrazolo[1,5-a]pyridine-2,3-dicarboxylic acid dimethyl ester as a crystalline solid.

¹H NMR (CDCl₃, TMS) δ: 3.93 (3H, s); 4.03 (3H, s); 7.52 (1H, dd); 8.08 (1H, dd); 8.66 (1H, dd).

A solution of 6-bromo-pyrazolo[1,5-a]pyridine-2,3-dicarboxylic acid dimethyl ester (2.4 mmol) in 50% aqueous sulfuric acid (20 mL) is heated at 80° C. for 3 h. The cooled mixture is treated with 5N NaOH solution followed by acidification with 2N aqueous HCl solution until pH 2-3 is reached. The formed precipitate is collected by filtration to provide sufficiently pure title compound as white solid.

¹H NMR: (DMSO-d₆, TMS) δ: 7.11 (1H, s); 7.35 (1H, dd); 7.82 (1H, d); 9.09 (1H, s).

### Preparation 9

### 6-Bromo-[1,2,4]triazolo[1,5-a]pyrimidine-2-carboxylic acid

Bromomalonaldehyde (0.46 g, 3.09 mmol) is added to a solution of 5-amino-1,2,4-triazole-3-carboxylic acid (0.42 g, 3.09 mmol) in 5 mL of glacial acetic acid. The mixture is heated to 70° C. for 3 h, then evaporated to dryness to provide sufficiently pure title compound.

### Preparation 10

### 6-Bromo-oxazolo[4,5-b]pyridine-2-carboxylic acid

2-Amino-5-bromo-3-hydroxypyridine (200 mg, 1.06 mmol) is dissolved in 3 mL of dioxane, triethoxyacetic acid ethyl ester (0.5 mL) and a catalytic amount of p-toluenesulfonic acid are added. The mixture is heated to reflux for 5 h, then cooled, diluted with DCM and washed with saturated sodium bicarbonate solution. The organic phase is dried over anhydrous sodium sulfate and evaporated to dryness. The residue is purified by flash column chromatography to afford 6-bromo-oxazolo[4,5-b]pyridine-2-carboxylic acid ethyl ester which is dissolved in a mixture of THF (2 mL) and water (0.5 mL). Lithium hydroxide (25 mg, 1 mmol) is added and the mixture is stirred at r.t. for 15 min., then neutralized with diluted hydrochloric acid until pH 7 is reached. The mixture is extracted with EtOAc, the organic phase is dried over anhydrous sodium sulfate and evaporated to dryness to afford sufficiently pure title compound.

### Preparation 11

### 6-Bromo-3-methyl-3H-imidazo[4,5-b]pyridine

5-Bromo-2-chloro-3-nitro-pyridine (1 g, 4.21 mmol) is dissolved in THF (5 mL) and an aqueous solution of methylamine (5 mL, 4.21 mmol) is added. The mixture is stirred at room temperature for 12 h. The mixture is then partitioned between saturated aqueous NaHCO₃ solution and dichloromethane. The organic phase is separated, dried over anhydrous Na₂SO₄ and evaporated to dryness to give sufficiently pure (5-Bromo-3-nitro-pyridin-2-yl)-methyl-amine.

SnCl₂*2H₂O (4.71 g, 20.9 mmol) is added to a solution of (5-bromo-3-nitro-pyridin-2-yl)-methyl-amine (0.97 g, 4.2 mmol) in EtOAc and the mixture is heated to reflux for 3.5 h. The mixture is then cooled, diluted with saturated aqueous NaHCO₃ solution and extracted with EtOAc. The organic phase is dried over anhydrous Na₂SO₄ and evaporated to dryness to give sufficiently pure 5-Bromo-N*2*-methyl-pyridine-2,3-diamine.

5-Bromo-N*2*-methyl-pyridine-2,3-diamine (0.85 g, 4.55 mmol) is dissolved in trimethylorthoformate (12 mL) and the solution is heated to reflux for 3 h, then cooled and evaporated to dryness. The solid residue is purified by flash column chromatography to give the title compound as a yellow oil.

### Example 1

### 6-Phenylethynyl-pyrazolo[1,5-a]pyrimidine

According to General Procedure 1,6-bromo-pyrazolo[1,5-a]pyrimidine is reacted with phenylacetylene to provide the title compound in good yield.

¹H NMR (CDCL3), δ: 6.74 (d, 1H), 7.37-7.41 (m, 3H), 7.54-7.59 (m, 2H), 8.17 (d, 1H), 8.57 (d, 1H), 8.83 (d, 1H).

LC/MS (M+H)⁺=220

### Example 2

### 6-(3,5-Dichloro-phenylethynyl)-pyrazolo[1,5-a]pyrimidine

According to General Procedure 1,6-bromo-pyrazolo[1,5-a]pyrimidine is reacted with 3,5-dichlorophenylacetylene to provide the title compound in good yield.

¹H NMR (CDCL3), δ: 6.76 (d, 1H), 7.36-7.45 (m, 3H), 8.19 (d, 1H), 8.54 (d, 1H), 8.84 (d, 1H)

LC/MS (M+H)⁺=289

### Example 3

### 6-(3-Fluoro-phenylethynyl)-pyrazolo[1,5-a]pyrimidine

According to General Procedure 1,6-bromo-pyrazolo[1,5-a]pyrimidine is reacted with 3-fluorophenylacetylene to provide the title compound in good yield.

¹H NMR (CDCL3), δ: 6.75 (d, 1H), 7.05-7.38 (m, 4H), 8.18 (d, 1H), 8.56 (d, 1H), 8.84 (d, 1H)

LC/MS (M+H)⁺=238

### Example 4

### 6-(4-Fluoro-phenylethynyl)-pyrazolo[1,5-a]pyrimidine

According to General Procedure 1,6-bromo-pyrazolo[1,5-a]pyrimidine is reacted with 4-fluorophenylacetylene to provide the title compound in good yield.

¹H NMR (CDCL3), δ: 6.73 (d, 1H), 7.09 (m, 2H), 7.55 (m, 2H), 8.17 (d, 1H), 8.55 (d, 1H), 8.82 (d, 1H).

LC/MS (M+H)⁺=238

### Example 5

### 6-(2-Fluoro-phenylethynyl)-pyrazolo[1,5-a]pyrimidine

According to General Procedure 1,6-bromo-pyrazolo[1,5-a]pyrimidine is reacted with 2-fluorophenylacetylene to provide the title compound in good yield.

¹H NMR (CDCL3), δ: 6.75 (d, 1H), 7.10-7.59 (m, 4H), 8.18 (d, 1H), 8.59 (d, 1H), 8.87 (d, 1H)

LC/MS (M+H)⁺=238

### Example 6

### 6-Thiophen-3-ylethynyl-pyrazolo[1,5-a]pyrimidine

According to General Procedure 1,6-bromo-pyrazolo[1,5-a]pyrimidine is reacted with 3-ethynylthiophene to provide the title compound in good yield.

¹H NMR (CDCL3), δ: 6.73 (d, 1H), 7.21 (m, 1H), 7.35 (m, 1H), 7.60 (t, 1H), 8.16 (d, 1H), 8.54 (d, 1H), 8.81 (d, 1H).

LC/MS (M+H)⁺=226

### Example 7

### 6-(3-Methyl-thiophen-2-ylethynyl)-pyrazolo[1,5-a]pyrimidine

According to General Procedure 1,6-bromo-pyrazolo[1,5-a]pyrimidine is reacted with 2-ethynyl-3-methylthiophene to provide the title compound in good yield.

¹H NMR (CDCL3), δ: 2.40 (s, 3H), 6.73 (d, 1H), 6.89 (d, 1H), 7.25 (d, 1H), 8.16 (d, 1H), 8.54 (d, 1H), 8.82 (d, 1H).

LC/MS (M+H)⁺=239

### Example 8

### 6-Cyclohex-1-enylethynyl-pyrazolo[1,5-a]pyrimidine

According to General Procedure 1,6-bromo-pyrazolo[1,5-a]pyrimidine is reacted with 1-ethynylcyclohexene to provide the title compound in good yield.

¹H NMR (CDCL3), δ: 1.56-1.78 (m, 4H), 2.15-2.30 (m, 4H), 6.28 (m, 1H), 6.68 (d, 1H), 8.11 (d, 1H), 8.45 (d, 1H), 8.70 (d, 1H).

LC/MS (M+H)⁺=224

### Example 9

### 6-p-Tolylethynyl-pyrazolo[1,5-a]pyrimidine

According to General Procedure 1,6-bromo-pyrazolo[1,5-a]pyrimidine is reacted with p-tolylacetylene to provide the title compound in good yield.

¹H NMR (CDCL3), δ: 2.38 (s, 3H), 6.73 (d, 1H), 7.19 (d, 2H), 7.45 (d, 2H), 8.15 (d, 1H), 8.56 (d, 1H), 8.81 (d, 1H).

LC/MS (M+H)⁺=234

### Example 10

### 6-(3,6-Dihydro-2H-thiopyran-4-ylethynyl)-pyrazolo[1,5-a]pyrimidine

According to General Procedure 1,6-bromo-pyrazolo[1,5-a]pyrimidine is reacted with 4-ethynyl-3,6-dihydro-2H-thiopyran to provide the title compound in good yield.

¹H NMR (CDCL3), δ: 2.52-2.56 (m, 2H), 2.80 (t, 2H), 3.30 (m, 2H), 6.42 (m, 1H), 6.70 (d, 1H), 8.14 (d, 1H), 8.45 (d, 1H), 8.71 (d, 1H).

LC/MS (M+H)⁺=242

### Example 11

### 6-(3,5-Difluoro-phenylethynyl)-pyrazolo[1,5-a]pyrimidine

According to General Procedure 1,6-bromo-pyrazolo[1,5-a]pyrimidine is reacted with 3,5-difluorophenylacetylene to provide the title compound in good yield.

¹H NMR (CDCL3), δ: 6.74 (d, 1H), 6.87 (m, 1H), 7.06-7.10 (m, 2H), 8.19 (d, 1H), 8.55 (d, 1H), 8.84 (d, 1H).

LC/MS (M+H)⁺=256

### Example 12

### 4-Pyrazolo[1,5-a]pyrimidin-6-ylethynyl-3,6-dihydro-2H-pyridine-1-carboxylic acid tert-butyl ester

According to General Procedure 1,6-bromo-pyrazolo[1,5-a]pyrimidine is reacted with 4-ethynyl-3,6-dihydro-2H-pyridine-1-carboxylic acid tert-butyl ester to provide the title compound in good yield.

¹H NMR (CDCL3), δ: 1.48 (s, 9H), 2.35 (m, 2H), 3.56 (t, 2H), 4.05 (m, 2H), 6.19 (m, 1H), 6.71 (d, 1H), 8.14 (d, 1H), 8.45 (d, 1H), 8.72 (d, 1H).

LC/MS (M+H)⁺=325

### Example 13

### 6-Thiophen-2-ylethynyl-pyrazolo[1,5-a]pyrimidine

According to General Procedure 1,6-bromo-pyrazolo[1,5-a]pyrimidine is reacted with 2-ethynylthiophene to provide the title compound in good yield.

¹H NMR (CDCL3), δ: 6.73 (d, 1H), 7.05 (t, 1H), 7.36 (m, 2H), 8.16 (d, 1H), 8.54 (d, 1H), 8.81 (d, 1H).

LC/MS (M+H)⁺=226

### Example 14

### 6-(3-Phenyl-prop-1-ynyl)-pyrazolo[1,5-a]pyrimidine

According to General Procedure 1,6-bromo-pyrazolo[1,5-a]pyrimidine is reacted with prop-2-ynylbenzene to provide the title compound in good yield.

¹H NMR (CDCL3), δ: 3.86 (s, 2H), 6.69 (d, 1H), 7.25-7.40 (m, 5H), 8.12 (d, 1H), 8.47 (d, 1H), 8.73 (d, 1H).

LC/MS (M+H)⁺=234

### Example 15

### Morpholin-4-yl-(6-phenylethynyl-pyrazolo[1,5-a]pyrimidin-2-yl)-methanone

According to General Procedure 2,6-bromo-pyrazolo[1,5-a]pyrimidine-2-carboxylic acid is reacted with morpholine to provide an amide, which is reacted with phenylacetylene according to General Procedure 1 to give the title compound in good overall yield.

¹H NMR (CDCL3), δ: 3.73-3.97 (m, 8H), 7.07 (s, 1H), 7.25-7.42 (m, 3H), 7.53-7.57 (m, 2H), 8.62 (d, 1H), 8.77 (d, 1H).

LC/MS (M+H)⁺=332

### Example 16

### (6-Phenylethynyl-pyrazolo[1,5-a]pyrimidin-2-yl)-piperidin-1-yl-methanone

According to General Procedure 2,6-bromo-pyrazolo[1,5-a]pyrimidine-2-carboxylic acid is reacted with piperidine to provide an amide, which is reacted with phenylacetylene according to General Procedure 1 to give the title compound in good overall yield.

¹H NMR (CDCL3), δ: 1.61-1.70 (m, 6H), 3.71-3.77 (m, 4H), 6.97 (s, 1H), 7.38-7.42 (m, 3H), 7.56-7.59 (m, 2H), 8.60 (d, 1H), 8.79 (d, 1H).

LC/MS (M+H)⁺=331

### Example 17

### Azepan-1-yl-(6-phenylethynyl-pyrazolo[1,5-a]pyrimidin-2-yl)-methanone

According to General Procedure 2,6-bromo-pyrazolo[1,5-a]pyrimidine-2-carboxylic acid is reacted with azepane to provide an amide, which is reacted with phenylacetylene according to General Procedure 1 to give the title compound in good overall yield.

¹H NMR (CDCL3), δ: 1.56-1.87 (m, 8H), 3.71-3.75 (m, 4H), 7.00 (s, 1H), 7.38-7.42 (m, 3H), 7.54-7.59 (m, 2H), 8.60 (d, 1H), 8.79 (d, 1H).

LC/MS (M+H)⁺=345

### Example 18

### (6-Phenylethynyl-pyrazolo[1,5-a]pyrimidin-2-yl)-(4-phenyl-piperidin-1-yl)-methanone

According to General Procedure 2,6-bromo-pyrazolo[1,5-a]pyrimidine-2-carboxylic acid is reacted with 4-phenylpiperidine to provide an amide, which is reacted with phenylacetylene according to General Procedure 1 to give the title compound in good overall yield.

¹H NMR (CDCL3), δ: 1.74-2.05 (m, 4H), 2.78-2.97 (m, 2H), 3.24 (m, 1H), 4.56 (m, 1H), 4.94 (m, 1H), 7.04 (s, 1H), 7.24-7.42 (m, 8H), 7.55-7.58 (m, 2H), 8.61 (d, 1H), 8.80 (d, 1H)

LC/MS (M+H)⁺=407

### Example 19

### (6-Phenylethynyl-pyrazolo[1,5-a]pyrimidin-2-yl)-pyrrolidin-1-yl-methanone

According to General Procedure 2,6-bromo-pyrazolo[1,5-a]pyrimidine-2-carboxylic acid is reacted with pyrrolidine to provide an amide, which is reacted with phenylacetylene according to General Procedure 1 to give the title compound in good overall yield.

¹H NMR (CDCL3), δ: 1.92-2.02 (m, 4H), 3.72 (t, 2H), 3.92 (t, 2H), 7.17 (s, 1H), 7.38-7.42 (m, 3H), 7.54-7.59 (m, 2H), 8.60 (d, 1H), 8.78 (d, 1H).

LC/MS (M+H)⁺=317

### Example 20

### (1,3-Dihydro-isoindol-2-yl)-(6-phenylethynyl-pyrazolo[1,5-a]pyrimidin-2-yl)-methanone

According to General Procedure 2,6-bromo-pyrazolo[1,5-a]pyrimidine-2-carboxylic acid is reacted with 2,3-dihydro-1H-isoindole to provide an amide, which is reacted with phenylacetylene according to General Procedure 1 to give the title compound in good overall yield.

¹H NMR (CDCL3), δ: 5.08 (s, 2H), 5.38 (s, 2H), 7.21 (s, 1H), 7.38-7.42 (m, 3H), 7.55-7.59 (m, 2H), 8.64 (d, 1H), 8.87 (d, 1H).

LC/MS (M+H)⁺=365

### Example 21

### 1-(6-Phenylethynyl-pyrazolo[1,5-a]pyrimidine-2-carbonyl)-piperidin-4-one

According to General Procedure 2,6-bromo-pyrazolo[1,5-a]pyrimidine-2-carboxylic acid is reacted with piperidin-4-one to provide an amide, which is reacted with phenylacetylene according to General Procedure 1 to give the title compound in good overall yield.

¹H NMR (CDCL3), δ: 2.57-2.64 (m, 4H), 4.10 (t, 2H), 4.23 (t, 2H), 7.14 (s, 1H), 7.39-7.42 (m, 3H), 7.56-7.59 (m, 2H), 8.64 (d, 1H), 8.80 (d, 1H).

LC/MS (M+H)⁺=345

### Example 22

### 4-[2-(Piperidine-1-carbonyl)-pyrazolo[1,5-a]pyrimidin-6-ylethynyl]-3,6-dihydro-2H-pyridine-1-carboxylic acid tert-butyl ester

According to General Procedure 2,6-bromo-pyrazolo[1,5-a]pyrimidine-2-carboxylic acid is reacted with piperidine to provide an amide, which is reacted with 4-ethynyl-3,6-dihydro-2H-pyridine-1-carboxylic acid tert-butyl ester according to General Procedure 1 to give the title compound in good overall yield.

¹H NMR (CDCL3), δ: 1.47 (s, 9H), 1.57-1.1.69 (m, 6H), 2.35 (m, 2H), 3.55 (t, 2H), 3.65-3.75 (m, 4H), 4.04 (q, 2H), 6.20 (m, 1H), 6.94 (s, 1H), 8.48 (d, 1H), 8.68 (d, 1H).

LC/MS (M+H)⁺=436

### Example 23

### (4-Hydroxy-4-methyl-piperidin-1-yl)-(6-phenylethynyl-pyrazolo[1,5-a]pyrimidin-2-yl)-methanone

According to General Procedure 2,6-bromo-pyrazolo[1,5-a]pyrimidine-2-carboxylic acid is reacted with 4-hydroxy-4-methylpiperidine to provide an amide, which is reacted with phenylacetylene according to General Procedure 1 to give the title compound in good overall yield.

¹H NMR (CDCL3), δ: 1.32 (s, 3H), 1.57-1.74 (m, 4H), 3.38 (m, 1H), 3.61 (m, 1H), 4.10 (m, 1H), 4.43 (m, 1H), 7.00 (s, 1H), 7.39-7.42 (m, 3H), 7.56-7.59 (m, 2H), 8.61 (d, 1H), 8.80 (d, 1H).

LC/MS (M+H)⁺=361

### Example 24

### (4-Hydroxy-piperidin-1-yl)-(6-phenylethynyl-pyrazolo[1,5-a]pyrimidin-2-yl)-methanone

According to General Procedure 2,6-bromo-pyrazolo[1,5-a]pyrimidine-2-carboxylic acid is reacted with 4-hydroxypiperidine to provide an amide, which is reacted with phenylacetylene according to General Procedure 1 to give the title compound in good overall yield.

¹H NMR (CDCL3), δ: 1.57-1.70 (m, 2H), 1.85-2.10 (m, 2H), 3.42-3.58 (m, 2H), 4.03 (m, 1H), 4.13-4.28 (m, 2H), 7.01 (s, 1H), 7.39-7.42 (m, 3H), 7.56-7.59 (m, 2H), 8.61 (d, 1H), 8.79 (d, 1H).

LC/MS (M+H)⁺=347

### Example 25

### (1-Methyl-3,4-dihydro-1H-isoquinolin-2-yl)-(6-phenylethynyl-pyrazolo[1,5-a]pyrimidin-2-yl)-methanone

According to General Procedure 2,6-bromo-pyrazolo[1,5-a]pyrimidine-2-carboxylic acid is reacted with 1-methyl-1,2,3,4-tetrahydro-isoquinoline to provide an amide, which is reacted with phenylacetylene according to General Procedure 1 to give the title compound in good overall yield.

¹H NMR (CDCL3), δ: 1.61-1.69 (m, 3H), 2.83 (m, 1H), 3.05-3.40 (m, 1H), 3.57 (m, 1H), 4.48 and 4.84 (both dd, 1H together), 5.58 and 5.82 (both q, 1H together), 7.02 (s, 1H), 7.06-7.21 (m, 3H), 7.39-7.42 (m, 4H), 7.55-7.58 (m, 2H), 8.62 (d, 1H), 8.82 (d, 1H).

LC/MS (M+H)⁺=393

### Example 26

### 6-Phenylethynyl-pyrazolo[1,5-a]pyrimidine-2-carboxylic acid cyclohexylamide

According to General Procedure 2,6-bromo-pyrazolo[1,5-a]pyrimidine-2-carboxylic acid is reacted with cyclohexylamine to provide an amide, which is reacted with phenylacetylene according to General Procedure 1 to give the title compound in good overall yield.

¹H NMR (CDCL3), δ: 1.15-1.39 (m, 5H), 1.57-1.72 (m, 4H), 1.97 (m, 2H), 3.94 (m, 1H), 6.90 (d, 1H), 7.24 (s, 1H), 7.32-7.34 (m, 3H), 7.48-7.50 (m, 2H), 8.52 (d, 1H), 8.65 (d, 1H)

LC/MS (M+H)⁺=345

### Example 27

### 6-Phenylethynyl-pyrazolo[1,5-a]pyrimidine-2-carboxylic acid cyclopentylamide

According to General Procedure 2,6-bromo-pyrazolo[1,5-a]pyrimidine-2-carboxylic acid is reacted with cyclopentylamine to provide an amide, which is reacted with phenylacetylene according to General Procedure 1 to give the title compound in good overall yield.

¹H NMR (CDCL3), δ: 1.55-1.77 (m, 6H), 2.09 (m, 2H), 4.41 (m, 1H), 6.99 (d, 1H), 7.25 (s, 1H), 7.38-7.40 (m, 3H), 7.54-7.57 (m, 2H), 8.58 (d, 1H), 8.70 (d, 1H).

LC/MS (M+H)⁺=331

### Example 28

### 2-(4-Fluoro-phenyl)-6-phenylethynyl-pyrazolo[1,5-a]pyrimidine

According to General Procedure 1,6-bromo-2-(4-fluoro-phenyl)-pyrazolo[1,5-a]pyrimidine is reacted with phenylacetylene to provide the title compound in good yield.

¹H NMR (CDCL3), δ: 6.96 (s, 1H), 7.13-7.21 (m, 2H), 7.38-7.41 (m, 3H), 7.55-7.58 (m, 2H), 7.93-8.00 (m, 2H), 8.56 (d, 1H), 8.79 (d, 1H).

LC/MS (M+H)⁺=314

### Example 29

### 6-Phenylethynyl-pyrazolo[1,5-a]pyridine

According to General Procedure 1,6-bromo-pyrazolo[1,5-a]pyridine is reacted with phenylacetylene to provide the title compound in good yield.

¹H NMR (CDCL3), δ: 6.54 (d, 1H), 7.19 (dd, 1H), 7.35-7.38 (m, 3H), 7.53-7.58 (m, 3H), 8.00 (d, 1H), 8.68 (d, 1H).

LC/MS (M+H)⁺=219

### Example 30

### 6-Cyclohex-1-enylethynyl-pyrazolo[1,5-a]pyridine

According to General Procedure 1,6-bromo-pyrazolo[1,5-a]pyridine is reacted with 1-ethynylcyclohexene to provide the title compound in good yield.

¹H NMR (CDCL3), δ: 1.60-1.74 (m, 4H), 2.14-2.23 (m, 4H), 6.24 (m, 1H), 6.49 (d, 1H), 7.08 (dd, 1H), 7.44 (d, 1H), 7.95 (d, 1H), 8.55 (d, 1H).

LC/MS (M+H)⁺=223

### Example 31

### 6-p-Tolylethynyl-pyrazolo[1,5-a]pyridine

According to General Procedure 1,6-bromo-pyrazolo[1,5-a]pyridine is reacted with p-tolylacetylene to provide the title compound in good yield.

¹H NMR (CDCL3), δ: 2.38 (s, 3H), 6.53 (d, 1H), 7.17 (d, 2H), 7.44 (d, 2H), 7.50 (dd, 1H), 7.98 (d, 1H), 8.68 (d, 1H).

LC/MS (M+H)⁺=233

### Example 32

### (6-Phenylethynyl-pyrazolo[1,5-a]pyridin-2-yl)-piperidin-1-yl-methanone

According to General Procedure 2,6-bromo-pyrazolo[1,5-a]pyridine-2-carboxylic acid is reacted with piperidine to provide an amide, which is reacted with phenylacetylene according to General Procedure 1 to give the title compound in good overall yield.

¹H NMR (CDCL3), δ: 1.57-1.69 (m, 6H), 3.77 (m, 4H), 6.81 (s, 1H), 7.22 (dd, 1H), 7.35-7.38 (m, 3H), 7.50-7.57 (m, 3H), 8.62 (d, 1H).

LC/MS (M+H)⁺=330

### Example 33

### 6-Phenylethynyl-[1,2,4]triazolo[1,5-a]pyrimidine

According to General Procedure 1,6-bromo-[1,2,4]triazolo[1,5-a]pyrimidine is reacted with phenylacetylene to provide the title compound in good yield.

¹H NMR (CDCL3), δ: 7.40-7.43 (m, 3H), 7.56-7.60 (m, 2H), 8.55 (s, 1H), 8.93 (d, 1H), 8.99 (d, 1H).

LC/MS (M+H)⁺=221

### Example 34

### 6-Thiophen-2-ylethynyl-[1,2,4]triazolo[1,5-a]pyrimidine

According to General Procedure 1,6-bromo-[1,2,4]triazolo[1,5-a]pyrimidine is reacted with 2-ethynylthiophene to provide the title compound in good yield.

¹H NMR (CDCL3), δ: 7.07 (dd, 1H), 7.41 (m, 2H), 8.55 (s, 1H), 8.91 (d, 1H), 8.98 (d, 1H)

LC/MS (M+H)⁺=227

### Example 35

### 6-p-Tolylethynyl-[1,2,4]triazolo[1,5-a]pyrimidine

According to General Procedure 1,6-bromo-[1,2,4]triazolo[1,5-a]pyrimidine is reacted with p-tolylacetylene to provide the title compound in good yield.

¹H NMR (CDCL3), δ: 2.40 (s, 3H), 7.21 (d, 2H), 7.46 (d, 2H), 8.54 (s, 1H), 8.91 (d, 1H), 8.97 (d, 1H).

LC/MS (M+H)⁺=235

### Example 36

### (6-Phenylethynyl-[1,2,4]triazolo[1,5-a]pyrimidin-2-yl)-piperidin-1-yl-methanone

According to General Procedure 2,6-bromo-[1,2,4]triazolo[1,5-a]pyrimidine-2-carboxylic acid is reacted with piperidine to provide an amide, which is reacted with phenylacetylene according to General Procedure 1 to give the title compound in good overall yield.

¹H NMR (CDCL3), δ: 1.63-1.70 (m, 6H), 3.67-3.79 (m, 4H), 7.40-7.43 (m, 3H), 7.58-7.61 (m, 2H), 8.97 (m, 2H).

LC/MS (M+H)⁺=332

### Example 37

### 6-Phenylethynyl-[1,2,4]triazolo[1,5-a]pyridine

According to General Procedure 1,6-bromo-[1,2,4]triazolo[1,5-a]pyridine is reacted with phenylacetylene to provide the title compound in good yield.

¹H NMR (CDCL3), δ: 7.37-7.40 (m, 3H), 7.54-7.61 (m, 2H), 7.62 (dd, 1H), 7.75 (d, 1H), 8.38 (s, 1H), 8.79 (s, 1H).

LC/MS (M+H)⁺=220

### Example 38

### 6-Phenylethynyl-thiazolo[4,5-b]pyridine

According to General Procedure 1,6-bromo-thiazolo[4,5-b]pyridine is reacted with phenylacetylene to provide the title compound in good yield.

¹H NMR (CDCL3), δ: 7.38-7.40 (m, 3H), 7.55-7.61 (m, 2H), 8.46 (d, 1H), 8.93 (d, 1H), 9.32 (s, 1H).

LC/MS (M+H)⁺=237

### Example 39

### 7-Phenylethynyl-pyrido[2,3-b]pyrazine

According to General Procedure 1,7-bromo-pyrido[2,3-b]pyrazine is reacted with phenylacetylene to provide the title compound in good yield.

¹H NMR (CDCL3), δ: 7.40-7.46 (m, 3H), 7.60-7.65 (m, 2H), 8.56 (d, 1H), 8.97 (d, 1H), 9.04 (d, 1H), 9.26 (d, 1H).

LC/MS (M+H)⁺=232

### Example 40

### 7-Cyclohex-1-enylethynyl-pyrido[2,3-b]pyrazine

According to General Procedure 1,7-bromo-pyrido[2,3-b]pyrazine is reacted with 1-ethynylcyclohexene to provide the title compound in good yield.

¹H NMR (CDCL3), δ: 1.60-1.76 (m, 4H), 2.16-2.29 (m, 4H), 6.38 (m, 1H), 8.42 (d, 1H), 8.93 (d, 1H), 9.00 (d, 1H), 9.14 (d, 1H).

LC/MS (M+H)⁺=236

### Example 41

### 3-Phenylethynyl-[1,5]naphthyridine

According to General Procedure 1,3-bromo-[1,5]naphthyridine is reacted with phenylacetylene to provide the title compound in good yield.

¹H NMR (CDCL3), δ: 7.39-7.41 (m, 3H), 7.61-7.64 (m, 3H), 8.39 (dd, 1H), 8.51 (d, 1H), 9.00 (dd, 1H), 9.05 (d, 1H).

LC/MS (M+H)⁺=231

### Example 42

### 6-Phenylethynyl-oxazolo[4,5-b]pyridine

According to General Procedure 1,6-bromo-oxazolo[4,5-b]pyridine is reacted with phenylacetylene to provide the title compound in good yield.

¹H NMR (CDCl₃), δ: 7.38-7.40 (m, 3H), 7.56-7.59 (m, 2H), 8.03 (d, 1H), 8.36 (s, 1H), 8.79 (d, 1H).

LC/MS (M+H)⁺=221

### Example 43

### (6-Phenylethynyl-oxazolo[4,5-b]pyridin-2-yl)-piperidin-1-yl-methanone

According to General Procedure 2,6-bromo-oxazolo[4,5-b]pyridine-2-carboxylic acid is reacted with piperidine to provide an amide, which is reacted with phenylacetylene according to General Procedure 1 to give the title compound in good overall yield.

¹H NMR (CDCl₃), δ: 1.74 (m, 6H), 3.80 (m, 2H), 4.08 (m, 2H), 7.38-7.41 (m, 3H), 7.56-7.61 (m, 2H), 8.05 (d, 1H), 8.81 (d, 1H).

LC/MS (M+H)⁺=332

### Example 44

### 6-(3-Fluoro-phenylethynyl)-thiazolo[4,5-b]pyridine

According to General Procedure 1,6-bromothiazolo[4,5-b]pyridine is reacted with 3-fluorophenylacetylene to provide the title compound in good yield.

¹H NMR (DMSO-d₆), δ: 7.32-7.37 (m, 1H), 7.47-7.56 (m, 3H), 8.90 (s, 1H), 8.94 (s, 1H), 9.80 (s, 1H).

LC/MS (M+H)⁺=255

### Example 45

### 6-(2-Fluoro-phenylethynyl)-thiazolo[4,5-b]pyridine

According to General Procedure 1,6-bromothiazolo[4,5-b]pyridine is reacted with 2-fluorophenylacetylene to provide the title compound in good yield.

¹H NMR (DMSO-d₆), δ: 7.30-7.42 (m, 2H), 7.52-7.74 (m, 2H), 8.86 (d, 1H), 8.98 (d, 1H), 9.81 (s, 1H).

LC/MS (M+H)⁺=255

### Example 46

### 6-(4-Fluoro-phenylethynyl)-thiazolo[4,5-b]pyridine

According to General Procedure 1,6-bromothiazolo[4,5-b]pyridine is reacted with 4-fluorophenylacetylene to provide the title compound in good yield.

¹H NMR (CDCl₃), δ: 7.09 (t, 2H), 7.56 (m, 2H), 8.45 (s, 1H), 8.92 (s, 1H), 9.34 (s, 1H).

LC/MS (M+H)⁺=255

### Example 47

### 6-(3-Fluoro-phenylethynyl)-[1,2,4]triazolo[1,5-a]pyrimidine

According to General Procedure 1,6-bromo-[1,2,4]triazolo[1,5-a]pyrimidine is reacted with 3-fluorophenylacetylene to provide the title compound in good yield.

¹H NMR (DMSO-d₆), δ: 7.35-7.39 (m, 1H), 7.47-7.57 (m, 3H), 8.78 (s, 1H), 9.06 (s, 1H), 9.85 (s, 1H).

LC/MS (M+H)⁺=239

### Example 48

### 6-(2-Fluoro-phenylethynyl)-[1,2,4]triazolo[1,5-a]pyrimidine

According to General Procedure 1,6-bromo-[1,2,4]triazolo[1,5-a]pyrimidine is reacted with 2-fluorophenylacetylene to provide the title compound in good yield.

¹H NMR (DMSO-d₆), δ: 7.31-7.43 (m, 2H), 7.55 (m, 1H), 7.71 (m, 1H), 8.78 (s, 1H), 9.06 (s, 1H), 9.88 (s, 1H).

LC/MS (M+H)⁺=239

### Example 49

### 6-(4-Fluoro-phenylethynyl)-[1,2,4]triazolo[1,5-a]pyrimidine

According to General Procedure 1,6-bromo-[1,2,4]triazolo[1,5-a]pyrimidine is reacted with 4-fluorophenylacetylene to provide the title compound in good yield.

¹H NMR (DMSO-d₆), δ: 7.35 (t, 2H), 7.70 (m, 2H), 8.77 (s, 1H), 9.05 (s, 1H), 9.82 (s, 1H).

LC/MS (M+H)⁺=239

### Example 50

### [6-(3-Fluoro-phenylethynyl)-[1,2,4]triazolo[1,5-a]pyrimidin-2-yl]-piperidin-1-yl-methanone

According to General Procedure 1, [6-bromo-[1,2,4]triazolo[1,5-a]pyrimidin-2-yl]-piperidin-1-yl-methanone is reacted with 3-fluorophenylacetylene to provide the title compound in good yield.

¹H NMR (CDCl₃), δ: 1.66-1.72 (m, 6H), 3.70 (m, 2H), 3.80 (m, 2H), 7.13-7.17 (m, 1H), 7.27-7.30 (m, 1H), 7.37-7.41 (m, 2H), 8.96 (d, 1H), 9.00 (d, 1H).

LC/MS (M+H)⁺=350

### Example 51

### [6-(2-Fluoro-phenylethynyl)-[1,2,4]triazolo[1,5-a]pyrimidin-2-yl]-piperidin-1-yl-methanone

According to General Procedure 1, [6-bromo-[1,2,4]triazolo[1,5-a]pyrimidin-2-yl]-piperidin-1-yl-methanone is reacted with 2-fluorophenylacetylene to provide the title compound in good yield.

¹H NMR (CDCl₃), δ: 1.62-1.74 (m, 6H), 3.70 (m, 2H), 3.80 (m, 2H), 7.15-7.22 (m, 2H), 7.43 (m, 1H), 7.57 (m, 1H), 8.98 (d, 1H), 9.01 (d, 1H).

LC/MS (M+H)⁺=350

### Example 52

### [6-(4-Fluoro-phenylethynyl)-[1,2,4]triazolo[1,5-a]pyrimidin-2-yl]-piperidin-1-yl-methanone

According to General Procedure 1, [6-bromo-[1,2,4]triazolo[1,5-a]pyrimidin-2-yl]-piperidin-1-yl-methanone is reacted with 4-fluorophenylacetylene to provide the title compound in good yield.

¹H NMR (CDCl₃), δ: 1.66-1.73 (m, 6H), 3.71 (m, 2H), 3.80 (m, 2H), 7.12 (t, 2H), 7.58 (m, 2H), 8.95 (s, 1H), 8.98 (s, 1H).

LC/MS (M+H)⁺=350

### Example 53

### 6-(3-Fluoro-phenylethynyl)-[1,2,4]triazolo[1,5-a]pyridine

According to General Procedure 1,6-bromo-[1,2,4]triazolo[1,5-a]pyridine is reacted with 3-fluorophenylacetylene to provide the title compound in good yield.

¹H NMR (DMSO-d₆), δ: 7.32-7.37 (m, 1H), 7.45-7.55 (m, 3H), 7.80 (d, 1H), 7.93 (d, 1H), 8.62 (s, 1H), 9.39 (s, 1H).

LC/MS (M+H)⁺=238

### Example 54

### 6-(2-Fluoro-phenylethynyl)-[1,2,4]triazolo[1,5-a]pyridine

According to General Procedure 1,6-bromo-[1,2,4]triazolo[1,5-a]pyridine is reacted with 2-fluorophenylacetylene to provide the title compound in good yield.

¹H NMR (DMSO-d₆), δ: 7.32 (t, 1H), 7.39 (t, 1H), 7.53 (m, 1H), 7.69 (m, 1H), 7.79 (d, 1H), 7.92 (d, 1H), 8.63 (s, 1H), 9.41 (s, 1H).

LC/MS (M+H)⁺=238

### Example 55

### 6-(4-Fluoro-phenylethynyl)-[1,2,4]triazolo[1,5-a]pyridine

According to General Procedure 1,6-bromo-[1,2,4]triazolo[1,5-a]pyridine is reacted with 4-fluorophenylacetylene to provide the title compound in good yield.

¹H NMR (DMSO-d₆), δ: 7.33 (t, 2H), 7.68 (m, 2H), 7.79 (d, 1H), 7.93 (d, 1H), 8.60 (s, 1H), 9.36 (s, 1H).

LC/MS (M+H)⁺=238

### Example 56

### [6-Phenylethynyl-[1,2,4]triazolo[1,5-a]pyridin-2-yl]-piperidin-1-yl-methanone

According to General Procedure 1, [6-bromo-[1,2,4]triazolo[1,5-a]pyridin-2-yl]-piperidin-1-yl-methanone is reacted with phenylacetylene to provide the title compound in good yield.

¹H NMR (CDCl₃), δ: 1.66-1.70 (m, 6H), 3.66 (m, 2H), 3.79 (m, 2H), 7.29-7.39 (m, 3H), 7.54-7.58 (m, 2H), 7.65 (d 1H), 7.74 (d, 1H), 8.76 (s, 1H).

LC/MS (M+H)⁺=331

### Example 57

### [6-(3-Fluoro-phenylethynyl)-[1,2,4]triazolo[1,5-a]pyridin-2-yl]-piperidin-1-yl-methanone

According to General Procedure 1, [6-bromo-[1,2,4]triazolo[1,5-a]pyridin-2-yl]-piperidin-1-yl-methanone is reacted with 3-fluorophenylacetylene to provide the title compound in good yield.

¹H NMR (DMSO-d₆), δ: 1.58-1.65 (m, 6H), 3.42 (m, 2H), 3.65 (m, 2H), 7.35 (m, 1H), 7.46-7.56 (m, 3H), 7.86 (d, 1H), 7.96 (d, 1H), 9.41 (s, 1H).

LC/MS (M+H)⁺=348

### Example 58

### [6-(2-Fluoro-phenylethynyl)-[1,2,4]triazolo[1,5-a]pyridin-2-yl]-piperidin-1-yl-methanone

According to General Procedure 1, [6-bromo-[1,2,4]triazolo[1,5-a]pyridin-2-yl]-piperidin-1-yl-methanone is reacted with 2-fluorophenylacetylene to provide the title compound in good yield.

¹H NMR (DMSO-d₆), δ: 1.49-1.65 (m, 6H), 3.42 (m, 2H), 3.66 (m, 2H), 7.30-7.42 (m, 2H), 7.54 (m, 1H), 7.70 (m, 1H), 7.86 (d, 1H), 7.95 (d, 1H), 9.43 (s, 1H).

LC/MS (M+H)⁺=348

### Example 59

### [6-(4-Fluoro-phenylethynyl)-[1,2,4]triazolo[1,5-a]pyridin-2-yl]-piperidin-1-yl-methanone

According to General Procedure 1, [6-bromo-[1,2,4]triazolo[1,5-a]pyridin-2-yl]-piperidin-1-yl-methanone is reacted with 4-fluorophenylacetylene to provide the title compound in good yield.

¹H NMR (DMSO-d₆), δ: 1.49-1.65 (m, 6H), 3.42 (m, 2H), 3.65 (m, 2H), 7.33 (t, 2H), 7.69 (m, 2H), 7.85 (d, 1H), 7.95 (d, 1H), 9.39 (s, 1H).

LC/MS (M+H)⁺=348

### Example 60

### 7-(3-Fluoro-phenylethynyl)-pyrido[2,3-b]pyrazine

According to General Procedure 1,7-bromo-pyrido[2,3-b]pyrazine is reacted with 3-fluorophenylacetylene to provide the title compound in good yield.

¹H NMR (DMSO-d₆), δ: 7.36-7.41 (m, 1H), 7.54-7.59 (m, 3H), 8.79 (s, 1H), 9.15 (d, 2H), 9.31 (s, 1H).

LC/MS (M+H)⁺=250

### Example 61

### 7-(2-Fluoro-phenylethynyl)-pyrido[2,3-b]pyrazine

According to General Procedure 1,7-bromo-pyrido[2,3-b]pyrazine is reacted with 2-fluorophenylacetylene to provide the title compound in good yield.

¹H NMR (CDCl₃), δ: 7.16-7.23 (m, 2H), 7.39-7.45 (m, 1H), 7.60-7.64 (m, 1H), 8.61 (s, 1H), 8.99 (s, 1H), 9.07 (s, 1H), 9.29 (s, 1H).

LC/MS (M+H)⁺=250

### Example 62

### 7-(4-Fluoro-phenylethynyl)-pyrido[2,3-b]pyrazine

According to General Procedure 1,7-bromo-pyrido[2,3-b]pyrazine is reacted with 4-fluorophenylacetylene to provide the title compound in good yield.

¹H NMR (CDCl₃), δ: 7.12 (t, 2H), 7.62 (m, 2H), 8.56 (s, 1H), 8.98 (s, 1H), 9.06 (s, 1H), 9.26 (s, 1H).

LC/MS (M+H)⁺=250

### Example 63

### [7-phenylethynyl-pyrido[2,3-b]pyrazin-3-yl]-piperidin-1-yl-methanone

According to General Procedure 1, [7-bromo-pyrido[2,3-b]pyrazin-3-yl]-piperidin-1-yl-methanone is reacted with phenylacetylene to provide the title compound in good yield.

LC/MS (M+H)⁺=343

### Example 64

### [7-(3-Fluoro-phenylethynyl)-pyrido[2,3-b]pyrazin-3-yl]-piperidin-1-yl-methanone

According to General Procedure 1, [7-bromo-pyrido[2,3-b]pyrazin-3-yl]-piperidin-1-yl-methanone is reacted with 3-fluorophenylacetylene to provide the title compound in good yield.

LC/MS (M+H)⁺=361

### Example 65

### [7-(2-Fluoro-phenylethynyl)-pyrido[2,3-b]pyrazin-3-yl]-piperidin-1-yl-methanone

According to General Procedure 1, [7-bromo-pyrido[2,3-b]pyrazin-3-yl]-piperidin-1-yl-methanone is reacted with 2-fluorophenylacetylene to provide the title compound in good yield.

LC/MS (M+H)⁺=361

### Example 66

### [7-(4-Fluoro-phenylethynyl)-pyrido[2,3-b]pyrazin-3-yl]-piperidin-1-yl-methanone

According to General Procedure 1, [7-bromo-pyrido[2,3-b]pyrazin-3-yl]-piperidin-1-yl-methanone is reacted with 4-fluorophenylacetylene to provide the title compound in good yield.

LC/MS (M+H)⁺=361

### Example 67

### 3-(3-Fluoro-phenylethynyl)-[1,5]naphthyridine

According to General Procedure 1,3-bromo-[1,5]naphthyridine is reacted with 3-fluorophenylacetylene to provide the title compound in good yield.

¹H NMR (CDCl₃), δ: 7.10-7.15 (m, 1H), 7.31-7.42 (m, 3H), 7.66 (t, 1H), 8.41 (d, 1H), 8.53 (s, 1H), 9.03 (d, 1H), 9.07 (s, 1H).

LC/MS (M+H)⁺=249

### Example 68

### 3-(2-Fluoro-phenylethynyl)-[1,5]naphthyridine

According to General Procedure 1,3-bromo-[1,5]naphthyridine is reacted with 2-fluorophenylacetylene to provide the title compound in good yield.

¹H NMR (CDCl₃), δ: 7.32-7.42 (m, 2H), 7.54-7.60 (m, 3H), 7.77 (m, 1H), 7.86 (m, 1H), 8.49 (d, 1H), 8.65 (s, 1H), 9.08 (d, 1H), 9.13 (s, 1H).

LC/MS (M+H)⁺=249

### Example 69

### 3-(4-Fluoro-phenylethynyl)-[1,5]naphthyridine

According to General Procedure 1,3-bromo-[1,5]naphthyridine is reacted with 4-fluorophenylacetylene to provide the title compound in good yield.

¹H NMR (CDCl₃), δ: 7.11 (t, 2H), 7.59-7.67 (m, 3H), 8.41 (d, 1H), 8.51 (s, 1H), 9.02 (d, 1H), 9.05 (s, 1H).

LC/MS (M+H)⁺=249

### Example 70

### 6-(4-Fluoro-phenylethynyl)-oxazolo[4,5-b]pyridine

According to General Procedure 1,6-bromo-oxazolo[4,5-b]pyridine is reacted with 4-fluorophenylacetylene to provide the title compound in good yield.

¹H NMR (CDCl₃), δ: 7.04-7.13 (m, 2H), 7.52-7.59 (m, 2H), 8.01 (d, 1H), 8.37 (s, 1H), 8.78 (d, 1H).

LC/MS (M+H)⁺=239

### Example 71

### 6-Cyclohex-1-enylethynyl-oxazolo[4,5-b]pyridine

According to General Procedure 1,6-bromo-oxazolo[4,5-b]pyridine is reacted with 1-ethynylcyclohexene to provide the title compound in good yield.

¹H NMR (CDCl₃), δ: 1.64-1.69 (m, 4H), 2.17-2.24 (m, 4H), 6.29 (m, 1H), 7.89 (s, 1H), 8.31 (s, 1H), 8.66 (s, 1H).

LC/MS (M+H)⁺=225

### Example 72

### (6-Cyclohex-1-enylethynyl-oxazolo[4,5-b]pyridin-2-yl)-piperidin-1-yl-methanone

According to General Procedure 2,6-bromo-oxazolo[4,5-b]pyridine-2-carboxylic acid is reacted with piperidine to provide an amide, which is reacted with 1-ethynylcyclohexene according to General Procedure 1 to give the title compound in good overall yield.

¹H NMR (CDCl₃), δ: 1.50-1.73 (m, 10H), 2.16-2.24 (m, 4H), 3.78 (m, 2H), 4.07 (m, 2H), 6.32 (m, 1H), 7.92 (d, 1H), 8.68 (d, 1H).

LC/MS (M+H)⁺=336

### Example 73

### 6-(m-Tolylethynyl)thiazolo[4,5-b]pyridine

According to General Procedure 1,6-bromothiazolo[4,5-b]pyridine is reacted with m-tolylacetylene to provide the title compound in good yield.

¹H NMR (CDCl₃), δ: 2.38 (s, 3H), 7.20-7.30 (m, 2H), 7.38-7.41 (m, 2H), 8.45 (s, 1H), 8.92 (s, 1H), 9.33 (s, 1H).

LC/MS (M+H)⁺=251

### Example 74

### 6-(p-Tolylethynyl)thiazolo[4,5-b]pyridine

According to General Procedure 1,6-bromothiazolo[4,5-b]pyridine is reacted with p-tolylacetylene to provide the title compound in good yield.

¹H NMR (CDCl₃), δ: 2.40 (s, 3H), 7.20 (d, 2H), 7.47 (d, 2H), 8.44 (s, 1H), 8.92 (s, 1H), 9.32 (s, 1H).

LC/MS (M+H)⁺=251

### Example 75

### 6-(o-Tolylethynyl)thiazolo[4,5-b]pyridine

According to General Procedure 1,6-bromothiazolo[4,5-b]pyridine is reacted with o-tolylacetylene to provide the title compound in good yield.

¹H NMR (CDCl₃), δ: 2.55 (s, 3H), 7.19-7.30 (m, 3H), 7.54 (d, 1H), 8.46 (s, 1H), 8.94 (s, 1H), 9.33 (s, 1H).

LC/MS (M+H)⁺=251

### Example 76

### 6-(Pyridin-4-ylethynyl)thiazolo[4,5-b]pyridine

According to General Procedure 1,6-bromothiazolo[4,5-b]pyridine is reacted with 4-ethynylpyridine to provide the title compound in good yield.

¹H NMR (DMSO-d₆), δ: 7.60 (d, 2H), 8.68 (d, 2H), 8.95 (s, 1H), 9.00 (s, 1H), 9.82 (s, 1H)

LC/MS (M+H)⁺=238

### Example 77

### 6-(Pyridin-3-ylethynyl)thiazolo[4,5-b]pyridine

According to General Procedure 1,6-bromothiazolo[4,5-b]pyridine is reacted with 3-ethynylpyridine to provide the title compound in good yield.

¹H NMR (DMSO-d₆), δ: 7.52 (m, 1H), 8.06 (m, 1H), 8.65 (m, 1H), 8.85 (m, 1H), 8.93 (s, 1H), 8.97 (s, 1H), 9.81 (s, 1H).

LC/MS (M+H)⁺=238

### Example 78

### 6-((2,6-Difluorophenyl)ethynyl)thiazolo[4,5-b]pyridine

According to General Procedure 1,6-bromothiazolo[4,5-b]pyridine is reacted with 2,6-difluorophenylacetylene to provide the title compound in good yield.

¹H NMR (DMSO-d₆), δ: 7.32 (m, 2H), 7.56-7.64 (m, 1H), 8.91 (s, 1H), 9.00 (s, 1H), 9.82 (s, 1H).

LC/MS (M+H)⁺=273

### Example 79

### 6-((2,4-Difluorophenyl)ethynyl)thiazolo[4,5-b]pyridine

According to General Procedure 1,6-bromothiazolo[4,5-b]pyridine is reacted with 2,4-difluorophenylacetylene to provide the title compound in good yield.

¹H NMR (CDCl₃), δ: 6.89-6.95 (m, 2H), 7.55 (m, 1H), 8.49 (s, 1H), 8.94 (s, 1H), 9.35 (s, 1H).

LC/MS (M+H)⁺=273

### Example 80

### 6-((3,5-Difluorophenyl)ethynyl)thiazolo[4,5-b]pyridine

According to General Procedure 1,6-bromothiazolo[4,5-b]pyridine is reacted with 3,5-difluorophenylacetylene to provide the title compound in good yield.

¹H NMR (CDCl₃), δ: 6.84-6.90 (m, 1H), 7.07-7.12 (m, 2H), 8.48 (s, 1H), 8.93 (s, 1H), 9.37 (s, 1H).

LC/MS (M+H)⁺=273

### Example 81

### 6-Phenylethynyl-2-piperidin-1-yl-thiazolo[4,5-b]pyridine

According to General Procedure 1,6-bromo-2-piperidin-1-yl-thiazolo[4,5-b]pyridine is reacted with phenylacetylene to provide the title compound in good yield.

¹H NMR (DMSO-d₆), δ: 1.71 (br.m, 6H), 3.68 (br. m, 4H), 7.32-7.36 (m, 3H), 7.49-7.54 (m, 2H), 7.95 (d, 1H), 8.55 (d, 1H).

LC/MS (M+H)⁺=320

### Example 82

### 6-(p-Tolylethynyl)-[1,2,4]triazolo[1,5-a]pyridine

According to General Procedure 1,6-bromo-[1,2,4]triazolo[1,5-a]pyridine is reacted with p-tolylacetylene to provide the title compound in good yield.

¹H NMR (CDCl₃), δ: 2.29 (s, 3H), 7.19 (d, 2H), 7.45 (d, 2H), 7.62 (d, 1H), 7.74 (d, 1H), 8.38 (s, 1H), 8.77 (s, 1H).

LC/MS (M+H)⁺=234

### Example 83

### 6-(o-Tolylethynyl)-[1,2,4]triazolo[1,5-a]pyridine

According to General Procedure 1,6-bromo-[1,2,4]triazolo[1,5-a]pyridine is reacted with o-tolylacetylene to provide the title compound in good yield.

¹H NMR (CDCl₃), δ: 2.53 (s, 3H), 7.19-7.32 (m, 3H), 7.52 (d, 1H), 7.64 (d, 1H), 7.75 (d, 1H), 8.39 (s, 1H), 8.79 (s, 1H).

LC/MS (M+H)⁺=234

### Example 84

### 2-Furan-2-yl-6-phenylethynyl-[1,2,4]triazolo[1,5-a]pyridine

According to General Procedure 1,6-bromo-2-furan-2-yl-[1,2,4]triazolo[1,5-a]pyridine is reacted with phenylacetylene to provide the title compound in good yield.

¹H NMR (CDCl₃), δ: 6.58 (m, 1H), 7.20 (d, 1H), 7.36-7.40 (m, 3H), 7.54-7.71 (m, 5H), 8.73 (s, 1H).

LC/MS (M+H)⁺=286

### Example 85

### 7-(p-Tolylethynyl)-pyrido[2,3-b]pyrazine

According to General Procedure 1,7-bromo-pyrido[2,3-b]pyrazine is reacted with p-tolylacetylene to provide the title compound in good yield.

¹H NMR (CDCl₃), δ: 2.41 (s, 3H), 7.23 (d, 2H), 7.52 (d, 2H), 8.55 (s, 1H), 8.97 (s, 1H), 9.04 (s, 1H), 9.26 (s, 1H).

LC/MS (M+H)⁺=246

### Example 86

### 7-(m-Tolylethynyl)-pyrido[2,3-b]pyrazine

According to General Procedure 1,7-bromo-pyrido[2,3-b]pyrazine is reacted with m-tolylacetylene to provide the title compound in good yield.

¹H NMR (CDCl₃), δ: 2.40 (s, 3H), 7.23-7.33 (m, 2H), 7.43-7.46 (m, 2H), 8.56 (s, 1H), 8.97 (s, 1H), 9.05 (s, 1H), 9.26 (s, 1H).

LC/MS (M+H)⁺=246

### Example 87

### 7-(o-Tolylethynyl)-pyrido[2,3-b]pyrazine

According to General Procedure 1,7-bromo-pyrido[2,3-b]pyrazine is reacted with o-tolylacetylene to provide the title compound in good yield.

¹H NMR (CDCl₃), δ: 2.59 (s, 3H), 7.22-7.35 (m, 3H), 7.59 (d, 1H), 8.57 (s, 1H), 8.98 (s, 1H), 9.05 (s, 1H), 9.27 (s, 1H).

LC/MS (M+H)⁺=246

### Example 88

### 7-(Pyridin-4-ylethynyl)pyrido[2,3-b]pyrazine

According to General Procedure 1,7-bromo-pyrido[2,3-b]pyrazine is reacted with 4-ethynylpyridine to provide the title compound in good yield.

¹H NMR (DMSO-d₆), δ: 7.66 (d, 2H), 8.72 (d, 2H), 8.88 (s, 1H), 9.15 (s, 1H), 9.19 (s, 1H), 9.35 (s, 1H).

LC/MS (M+H)⁺=233

### Example 89

### 7-(Pyridin-3-ylethynyl)pyrido[2,3-b]pyrazine

According to General Procedure 1,7-bromo-pyrido[2,3-b]pyrazine is reacted with 3-ethynylpyridine to provide the title compound in good yield.

¹H NMR (CDCl₃), δ: 7.37 (dd, 1H), 7.93 (d, 1H), 8.61 (s, 1H), 8.66 (d, 1H), 8.88 (s, 1H), 9.00 (s, 1H), 9.08 (s, 1H), 9.29 (s, 1H).

LC/MS (M+H)⁺=233

### Example 90

### 4-(Pyrido[2,3-b]pyrazin-7-ylethynyl)phenol

According to General Procedure 1,7-bromo-pyrido[2,3-b]pyrazine is reacted with p-ethynylphenol to provide the title compound in good yield.

¹H NMR (DMSO-d₆), δ: 6.86 (d, 2H), 7.51 (d, 2H), 8.67 (s, 1H), 9.11 (s, 1H), 9.14 (s, 1H), 9.26 (s, 1H), 10.14 (s, 1H).

LC/MS (M+H)⁺=247

### Example 91

### 7-((3,6-Dihydro-2H-pyran-4-yl)ethynyl)pyrido[2,3-b]pyrazine

According to General Procedure 1,7-bromo-pyrido[2,3-b]pyrazine is reacted with (3,6-dihydro-2H-pyran-4-y)lacetylene to provide the title compound in good yield.

¹H NMR (CDCl₃), δ: 2.42 (m, 2H), 3.87 (t, 2H), 4.30 (d, 2H), 6.36 (m, 1H), 8.47 (s, 1H), 8.96 (s, 1H), 9.04 (s, 1H), 9.17 (s, 1H).

LC/MS (M+H)⁺=238

### Example 92

### 2-Methoxy-7-(phenylethynyl)pyrido[2,3-b]pyrazine

According to General Procedure 1,7-bromo-pyrido[2,3-b]pyrazine is reacted with p-ethynylphenol to provide the title compound in good yield.

¹H NMR (DMSO-d₆), δ: 4.08 (s, 3H), 7.48-7.50 (m, 3H), 7.65-7.68 (m, 2H), 8.46 (s, 1H), 8.79 (s, 1H), 9.02 (s, 1H).

LC/MS (M+H)⁺=262

### Example 93

### 3-(p-Tolylethynyl)-[1,5]naphthyridine

According to General Procedure 1,3-bromo-[1,5]naphthyridine is reacted with p-tolylacetylene to provide the title compound in good yield.

¹H NMR (CDCl₃), δ: 2.41 (s, 3H), 7.19 (d, 2H), 7.52 (d, 2H), 7.64 (dd, 1H), 8.40 (d, 1H), 8.50 (s, 1H), 9.01 (d, 1H), 9.06 (s, 1H).

LC/MS (M+H)⁺=245

### Example 94

### 3-(o-Tolylethynyl)-[1,5]naphthyridine

According to General Procedure 1,3-bromo-[1,5]naphthyridine is reacted with o-tolylacetylene to provide the title compound in good yield.

¹H NMR (CDCl₃), δ: 2.58 (s, 3H), 7.21-7.36 (m, 3H), 7.64 (d, 1H), 7.67 (dd, 1H), 8.41 (d, 1H), 8.52 (s, 1H), 9.00 (d, 1H), 9.07 (s, 1H).

LC/MS (M+H)⁺=245

### Example 95

### 3-(m-Tolylethynyl)-[1,5]naphthyridine

According to General Procedure 1,3-bromo-[1,5]naphthyridine is reacted with m-tolylacetylene to provide the title compound in good yield.

¹H NMR (CDCl₃), δ: 2.40 (s, 3H), 7.22-7.32 (m, 2H), 7.43-7.46 (m, 2H), 7.65 (dd, 1H), 8.40 (d, 1H), 8.51 (s, 1H), 9.01 (d, 1H), 9.06 (s, 1H).

LC/MS (M+H)⁺=245

### Example 96

### 3-(2,4-Difluoro-phenylethynyl)-[1,5]naphthyridine

According to General Procedure 1,3-bromo-[1,5]naphthyridine is reacted with 2,4-difluorophenylacetylene to provide the title compound in good yield.

¹H NMR (CDCl₃), δ: 6.91-6.97 (m, 2H), 7.57-7.63 (m, 1H), 7.66 (dd, 1H), 8.41 (d, 1H), 8.54 (s, 1H), 9.02 (d, 1H), 9.07 (s, 1H).

LC/MS (M+H)⁺=267

### Example 97

### 3-(3,5-Difluoro-phenylethynyl)-[1,5]naphthyridine

According to General Procedure 1,3-bromo-[1,5]naphthyridine is reacted with 3,5-difluorophenylacetylene to provide the title compound in good yield.

¹H NMR (CDCl₃), δ: 6.86-6.91 (m, 1H), 7.12-7.17 (m, 2H), 7.67 (dd, 1H), 8.42 (d, 1H), 8.53 (s, 1H), 9.03 (d, 1H), 9.06 (s, 1H).

LC/MS (M+H)⁺=267

### Example 98

### 3-((4-(Trifluoromethyl)phenyl)ethynyl)-1,5-naphthyridine

According to General Procedure 1,3-bromo-[1,5]naphthyridine is reacted with 4-(trifluoromethyl)phenylacetylene to provide the title compound in good yield.

¹H NMR (CDCl₃), δ: 7.65-7.73 (m, 5H), 8.40 (d, 1H), 8.54 (s, 1H), 9.02 (d, 1H), 9.07 (s, 1H).

LC/MS (M+H)⁺=299

### Example 99

### 3-((3-(Trifluoromethyl)phenyl)ethynyl)-1,5-naphthyridine

According to General Procedure 1,3-bromo-[1,5]naphthyridine is reacted with 3-(trifluoromethyl)phenylacetylene to provide the title compound in good yield.

¹H NMR (CDCl₃), δ: 7.51-7.55 (m, 1H), 7.64-7.7.67 (m, 2H), 7.79 (dd, 1H), 7.88 (s, 1H), 8.41 (d, 1H), 8.53 (s, 1H), 9.02 (d, 1H), 9.07 (s, 1H).

LC/MS (M+H)⁺=299

### Example 100

### 3-(Pyridin-4-ylethynyl)-1,5-naphthyridine

According to General Procedure 1,3-bromo-[1,5]naphthyridine is reacted with 4-ethynylpyridine to provide the title compound in good yield.

¹H NMR (CDCl₃), δ: 7.48 (d, 2H), 7.69 (dd, 1H), 8.43 (d, 1H), 8.57 (s, 1H), 8.68 (d, 2H), 9.04 (d, 1H), 9.08 (s, 1H).

LC/MS (M+H)⁺=232

### Example 101

### 3-(Pyridin-3-ylethynyl)-1,5-naphthyridine

According to General Procedure 1,3-bromo-[1,5]naphthyridine is reacted with 3-ethynylpyridine to provide the title compound in good yield.

¹H NMR (CDCl₃), δ: 7.36 (dd, 1H), 7.68 (dd, 1H), 7.91 (d, 1H), 8.42 (d, 1H), 8.55 (s, 1H), 8.63 (s, 1H), 8.71 (s, 2H), 9.03 (d, 1H), 9.08 (s, 1H).

LC/MS (M+H)⁺=232

### Example 102

### 5-((1,5-Naphthyridin-3-yl)ethynyl)-N-methylpyridin-2-amine

According to General Procedure 1,3-bromo-[1,5]naphthyridine is reacted with 5-ethynyl-N-methylpyridin-2-amine to provide the title compound in good yield.

¹H NMR (CDCl₃), δ: 2.98 (d, 3H), 4.84 (br.s, 1H), 6.41 (d, 1H), 7.61-7.70 (m, 2H), 8.39 (m, 2H), 8.46 (s, 1H), 9.00 (d, 1H), 9.03 (s, 1H).

LC/MS (M+H)⁺=261

### Example 103

### 5-((1,5-Naphthyridin-3-yl)ethynyl)-N-methylpyrimidin-2-amine

According to General Procedure 1,3-bromo-[1,5]naphthyridine is reacted with 5-ethynyl-N-methylpyrimidin-2-amine to provide the title compound in good yield.

¹H NMR (DMSO-d₆), δ: 3.35 (s, 3H), 7.76-7.83 (m, 2H), 8.46 (d, 1H), 8.53-8.8.62 (m, 3H), 9.05 (d, 1H), 9.03 (s, 1H).

LC/MS (M+H)⁺=262

### Example 104

### 3-Methyl-6-phenylethynyl-3H-imidazo[4,5-b]pyridine

According to General Procedure 1,6-bromo-3-methyl-3H-imidazo[4,5-b]pyridine is reacted with phenylacetylene to provide the title compound in good yield.

¹H NMR (CDCl₃), δ: 3.93 (s, 3H), 7.35-7.38 (m, 3H), 7.55-7.60 (m, 2H), 8.07 (s, 1H), 8.21 (d, 1H), 8.60 (d, 1H).

LC/MS (M+H)⁺=234

### Example 105

### 6-(3-Fluoro-phenylethynyl)-3-methyl-3H-imidazo[4,5-b]pyridine

According to General Procedure 1,6-bromo-3-methyl-3H-imidazo[4,5-b]pyridine is reacted with 3-fluorophenylacetylene to provide the title compound in good yield. ¹H NMR (CDCl₃), δ: 3.93 (s, 3H), 7.02-7.07 (m, 1H), 7.24-7.36 (m, 3H), 8.11 (s, 1H), 8.21 (d, 1H), 8.59 (d, 1H).

LC/MS (M+H)⁺=252

### Example 106

### MRZ-13820

### 6-(4-Fluoro-phenylethynyl)-3-methyl-3H-imidazo[4,5-b]pyridine

According to General Procedure 1,6-bromo-3-methyl-3H-imidazo[4,5-b]pyridine is reacted with 3-fluorophenylacetylene to provide the title compound in good yield. ¹H NMR (CDCl₃), δ: 3.93 (s, 3H), 7.06 (t, 2H), 7.51-7.58 (m, 2H), 8.09 (s, 1H), 8.20 (d, 1H), 8.57 (d, 1H).

LC/MS (M+H)⁺=252

### Example 107

### 7-((2,6-Difluorophenyl)ethynyl)pyrido[2,3-b]pyrazine

According to General Procedure 1,7-bromo-pyrido[2,3-b]pyrazine is reacted with 2,6-difluorophenylacetylene to provide the title compound in good yield.

¹H NMR (CDCL3), δ: 6.99-7.04 (m, 2H), 7.35-7.42 (m, 1H), 8.64 (d, 1H), 8.99 (d, 1H), 9.07 (d, 1H), 9.30 (d, 1H).

LC/MS (M+H)⁺=268

### Example 108

### 7-((3,5-Difluorophenyl)ethynyl)pyrido[2,3-b]pyrazine

According to General Procedure 1,7-bromo-pyrido[2,3-b]pyrazine is reacted with 3,5-difluorophenylacetylene to provide the title compound in good yield.

¹H NMR (CDCL3), δ: 6.88-6.94 (m, 1H), 7.14-7.17 (m, 2H), 8.60 (d, 1H), 9.00 (d, 1H), 9.08 (d, 1H), 9.26 (d, 1H).

LC/MS (M+H)⁺=268

### Example 109

### 7-((2,4-Difluorophenyl)ethynyl)pyrido[2,3-b]pyrazine

According to General Procedure 1,7-bromo-pyrido[2,3-b]pyrazine is reacted with 2,4-difluorophenylacetylene to provide the title compound in good yield.

¹H NMR (CDCL3), δ: 6.94-6.97 (m, 2H), 7.58-7.63 (m, 1H), 8.60 (d, 1H), 8.99 (d, 1H), 9.07 (d, 1H), 9.27 (d, 1H)

LC/MS (M+H)⁺=268

### Example 110

### 7-((2-Fluoro-4-methylphenyl)ethynyl)pyrido[2,3-b]pyrazine

According to General Procedure 1,7-bromo-pyrido[2,3-b]pyrazine is reacted with 2-fluoro-4-methylphenylacetylene to provide the title compound in good yield.

¹H NMR (CDCL3), δ: 2.41 (s, 3H), 6.98-7.02 (m, 2H), 7.48 (m, 1H), 8.59 (d, 1H), 8.98 (d, 1H), 9.06 (d, 1H), 9.28 (d, 1H)

LC/MS (M+H)⁺=264

### Example 111

### 7-((3-Chlorophenyl)ethynyl)pyrido[2,3-b]pyrazine

According to General Procedure 1,7-bromo-pyrido[2,3-b]pyrazine is reacted with 3-chlorophenylacetylene to provide the title compound in good yield.

¹H NMR (CDCL3), δ: 7.34-7.43 (m, 2H), 7.51 (m, 1H), 7.62, (s, 1H), 8.58 (d, 1H), 8.99 (d, 1H), 9.07 (d, 1H), 9.27 (d, 1H)

LC/MS (M+H)⁺=266

### Example 112

### 7-((4-Chlorophenyl)ethynyl)pyrido[2,3-b]pyrazine

According to General Procedure 1,7-bromo-pyrido[2,3-b]pyrazine is reacted with 4-chlorophenylacetylene to provide the title compound in good yield.

¹H NMR (CDCL3), δ: 7.40 (d, 2H), 7.56 (d, 1H), 8.57 (d, 1H), 8.98 (d, 1H), 9.06 (d, 1H), 9.26 (d, 1H)

LC/MS (M+H)⁺=266

### Example 113

### 7-((4-Fluoro-3-methylphenyl)ethynyl)pyrido[2,3-b]pyrazine

According to General Procedure 1,7-bromo-pyrido[2,3-b]pyrazine is reacted with 4-fluoro-3-methylphenylacetylene to provide the title compound in good yield.

¹H NMR (CDCL3), δ: 2.32 (s, 3H), 7.05 (m, 1H), 7.42-7.49 (m, 2H), 8.55 (d, 1H), 8.97 (d, 1H), 9.05 (d, 1H), 9.25 (d, 1H)

LC/MS (M+H)⁺=264

### Example 114

### 3-((2,6-Difluorophenyl)ethynyl)-[1,5]naphthyridine

According to General Procedure 1,3-bromo-[1,5]naphthyridine is reacted with 2,6-difluorophenylacetylene to provide the title compound in good yield.

¹H NMR (CDCl₃), δ: 6.97-7.03 (m, 2H), 7.33-7.40 (m, 1H), 7.65-7.69 (m, 1H), 8.42 (d, 1H), 8.59 (d, 1H), 9.02 (d, 1H), 9.10 (d, 1H).

LC/MS (M+H)⁺=267

### Example 115

### 3-((2-Fluoro-4-methylphenyl)ethynyl)-[1,5]naphthyridine

According to General Procedure 1,3-bromo-[1,5]naphthyridine is reacted with 2-fluoro-4-methylphenylacetylene to provide the title compound in good yield.

¹H NMR (CDCl₃), δ: 2.41 (s, 3H), 6.97-7.01 (m, 2H), 7.46-7.53 (m, 1H), 7.63-7.67 (m, 1H), 8.40 (d, 1H), 8.53 (d, 1H), 9.02 (d, 1H), 9.07 (d, 1H).

LC/MS (M+H)⁺=263

### Example 116

### 3-((4-Fluoro-3-methylphenyl)ethynyl)-[1,5]naphthyridine

According to General Procedure 1,3-bromo-[1,5]naphthyridine is reacted with 4-fluoro-3-methylphenylacetylene to provide the title compound in good yield.

¹H NMR (CDCl₃), δ: 2.31 (s, 3H), 7.02-7.06 (m, 1H), 7.41-7.49 (m, 2H), 7.63-7.67 (m, 1H), 8.40 (d, 1H), 8.49 (d, 1H), 9.00 (d, 1H), 9.04 (d, 1H).

LC/MS (M+H)⁺=263

### Example 117

### 3-((4-Chlorophenyl)ethynyl)-[1,5]naphthyridine

According to General Procedure 1,3-bromo-[1,5]naphthyridine is reacted with 4-chlorophenylacetylene to provide the title compound in good yield.

¹H NMR (CDCl₃), δ: 7.38 (d, 2H), 7.56 (d, 2H), 7.64-7.68 (m, 1H), 8.40 (d, 1H), 8.51 (d, 1H), 9.00 (d, 1H), 9.05 (d, 1H).

LC/MS (M+H)⁺=265

### Example 118

### 3-((3-Chlorophenyl)ethynyl)-[1,5]naphthyridine

According to General Procedure 1,3-bromo-[1,5]naphthyridine is reacted with 3-chlorophenylacetylene to provide the title compound in good yield.

¹H NMR (CDCl₃), δ: 7.32-7.40 (m, 2H), 7.51 (m, 1H), 7.62-7.68 (m, 2H), 8.41 (d, 1H), 8.53 (d, 1H), 9.02 (d, 1H), 9.06 (d, 1H).

LC/MS (M+H)⁺=265

### Example 119

### 5-((1,5-Naphthyridin-3-yl)ethynyl)pyridin-2-amine

According to General Procedure 1,3-bromo-[1,5]naphthyridine is reacted with 5-ethynylpyridin-2-amine to provide the title compound in good yield.

¹H NMR (DMSO-d₆), δ: 7.06 (d, 1H), 7.87-7.7.90 (m, 1H), 8.11 (d, 1H), 8.37 (d, 1H), 8.52 (d, 1H), 8.64 (d, 1H), 9.11 (br.s, 1H), 9.14 (d, 1H).

LC/MS (M+H)⁺=247

### Example 120

### 6-((4-Chlorophenyl)ethynyl)thiazolo[4,5-b]pyridine

According to General Procedure 1,6-bromo-thiazolo[4,5-b]pyridine is reacted with 4-chlorophenylacetylene to provide the title compound in good yield.

¹H NMR (DMSO-d₆), δ: 7.55 (d, 2H), 7.66 (d, 2H), 8.92 (d, 2H), 9.80 (s, 1H).

LC/MS (M+H)⁺=271

### Example 121

### 6-((4-Fluoro-3-methylphenyl)ethynyl)thiazolo[4,5-b]pyridine

According to General Procedure 1,6-bromo-thiazolo[4,5-b]pyridine is reacted with 4-fluoro-3-methylphenylacetylene to provide the title compound in good yield.

¹H NMR (CDCl₃), δ: 7.02 (m, 1H), 7.36-7.43 (m, 2H), 8.44 (d, 1H), 8.90 (d, 1H), 9.33 (s, 1H)

LC/MS (M+H)⁺=269

TABLE 1

| | |
|---|---|
| | I |
| 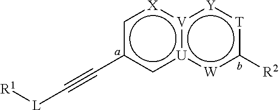 | |

| Example | | | | | | | | | |
|---|---|---|---|---|---|---|---|---|---|
| No. | R¹ | R² | L | T | U | V | W | X | Y |
| 1 | Ph | H | bond | bond | N | C | N | N | CH |
| 2 | 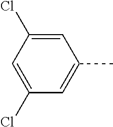 | H | bond | bond | N | C | N | N | CH |
| 3 | 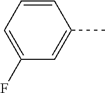 | H | bond | bond | N | C | N | N | CH |
| 4 | 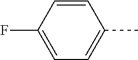 | H | bond | bond | N | C | N | N | CH |
| 5 | 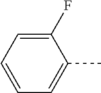 | H | bond | bond | N | C | N | N | CH |
| 6 | 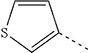 | H | bond | bond | N | C | N | N | CH |
| 7 | 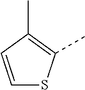 | H | bond | bond | N | C | N | N | CH |
| 8 | 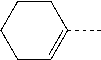 | H | bond | bond | N | C | N | N | CH |
| 9 | 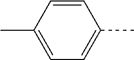 | H | bond | bond | N | C | N | N | CH |
| 10 | 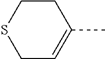 | H | bond | bond | N | C | N | N | CH |
| 11 | 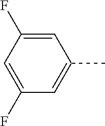 | H | bond | bond | N | C | N | N | CH |
| 12 | 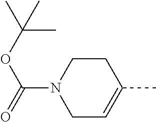 | H | bond | bond | N | C | N | N | CH |
| 13 | 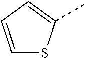 | H | bond | bond | N | C | N | N | CH |
| 14 | Ph | H | CH₂ | bond | N | C | N | N | CH |
| 15 | Ph | 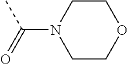 | bond | bond | N | C | N | N | CH |
| 16 | Ph | 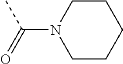 | bond | bond | N | C | N | N | CH |
| 17 | Ph | 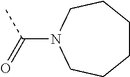 | bond | bond | N | C | N | N | CH |
| 18 | Ph | 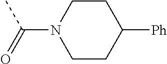 | bond | bond | N | C | N | N | CH |
| 19 | Ph | 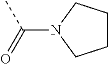 | bond | bond | N | C | N | N | CH |
| 20 | Ph | 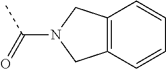 | bond | bond | N | C | N | N | CH |
| 21 | Ph | 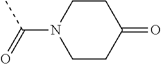 | bond | bond | N | C | N | N | CH |
| 22 | 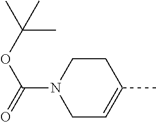 | 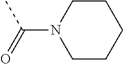 | bond | bond | N | C | N | N | CH |
| 23 | Ph | 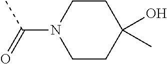 | bond | bond | N | C | N | N | CH |
| 24 | Ph | 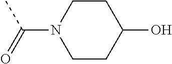 | bond | bond | N | C | N | N | CH |
| 25 | Ph | 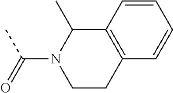 | bond | bond | N | C | N | N | CH |
| 26 | Ph | 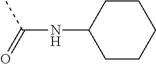 | bond | bond | N | C | N | N | CH |
| 27 | Ph | 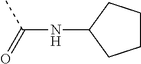 | bond | bond | N | C | N | N | CH |
| 28 | Ph | 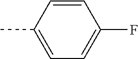 | bond | bond | N | C | N | N | CH |
| 29 | Ph | H | bond | bond | N | C | N | CH | CH |
| 30 | 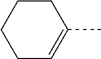 | H | bond | bond | N | C | N | CH | CH |
| 31 | 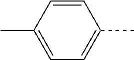 | H | bond | bond | N | C | N | CH | CH |
| 32 | Ph | 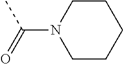 | bond | bond | N | C | N | CH | CH |
| 33 | Ph | H | bond | bond | N | C | N | N | N |
| 34 | 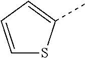 | H | bond | bond | N | C | N | N | N |
| 35 | 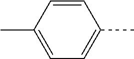 | H | bond | bond | N | C | N | N | N |
| 36 | Ph | 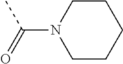 | bond | bond | N | C | N | N | N |
| 37 | Ph | H | bond | bond | N | C | N | CH | N |
| 38 | Ph | H | bond | bond | C | C | S | N | N |
| 39 | Ph | H | bond | CH | C | C | N | N | N |
| 40 | 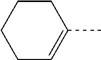 | H | bond | CH | C | C | N | N | N |
| 41 | Ph | H | bond | CH | C | C | N | N | CH |
| 42 | Ph | H | bond | bond | C | C | O | N | N |
| 43 | Ph | 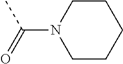 | bond | bond | C | C | O | N | N |
| 44 | 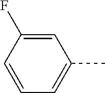 | H | bond | bond | C | C | S | N | N |
| 45 | 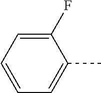 | H | bond | bond | C | C | S | N | N |
| 46 | 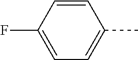 | H | bond | bond | C | C | S | N | N |
| 47 | 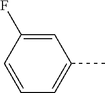 | H | bond | bond | N | C | N | N | N |
| 48 | 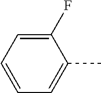 | H | bond | bond | N | C | N | N | N |
| 49 | 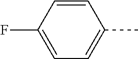 | H | bond | bond | N | C | N | N | N |
| 50 | 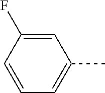 | 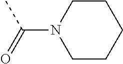 | bond | bond | N | C | N | N | N |
| 51 | 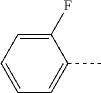 | 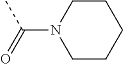 | bond | bond | N | C | N | N | N |
| 52 | 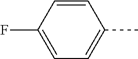 | 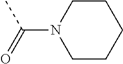 | bond | bond | N | C | N | N | N |
| 53 | 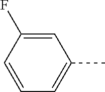 | H | bond | bond | N | C | N | C | N |
| 54 | 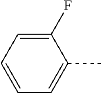 | H | bond | bond | N | C | N | C | N |
| 55 | 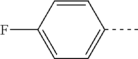 | H | bond | bond | N | C | N | C | N |
| 56 | Ph | 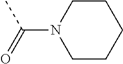 | bond | bond | N | C | N | C | N |
| 57 | 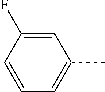 | 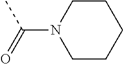 | bond | bond | N | C | N | C | N |
| 58 | 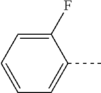 | 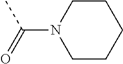 | bond | bond | N | C | N | C | N |
| 59 | 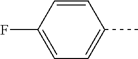 | 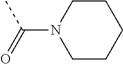 | bond | bond | N | C | N | C | N |
| 60 | 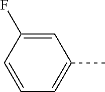 | H | bond | CH | C | C | N | N | N |
| 61 | 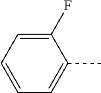 | H | bond | CH | C | C | N | N | N |
| 62 | 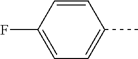 | H | bond | CH | C | C | N | N | N |
| 63 | Ph | 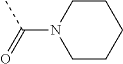 | bond | CH | C | C | N | N | N |
| 64 | 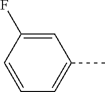 | 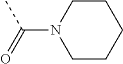 | bond | CH | C | C | N | N | N |
| 65 | 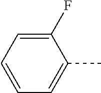 | 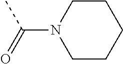 | bond | CH | C | C | N | N | N |
| 66 | 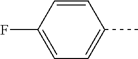 | 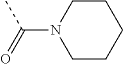 | bond | CH | C | C | N | N | N |
| 67 | 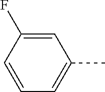 | H | bond | CH | C | C | N | N | CH |
| 68 | 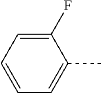 | H | bond | CH | C | C | N | N | CH |
| 69 | 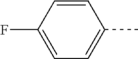 | H | bond | CH | C | C | N | N | CH |
| 70 | 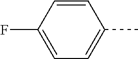 | H | bond | bond | C | C | O | N | N |
| 71 | 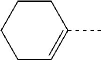 | H | bond | bond | C | C | O | N | N |
| 72 | 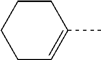 | 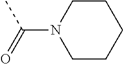 | bond | bond | C | C | O | N | N |
| 73 | 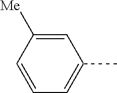 | H | bond | bond | C | C | S | N | N |
| 74 | 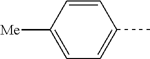 | H | bond | bond | C | C | S | N | N |
| 75 | 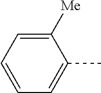 | H | bond | bond | C | C | S | N | N |
| 76 | 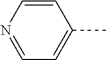 | H | bond | bond | C | C | S | N | N |
| 77 | 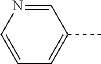 | H | bond | bond | C | C | S | N | N |
| 78 | 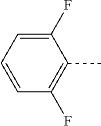 | H | bond | bond | C | C | S | N | N |
| 79 | 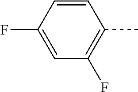 | H | bond | bond | C | C | S | N | N |
| 80 | 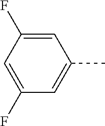 | H | bond | bond | C | C | S | N | N |
| 81 | Ph | 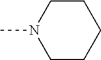 | bond | bond | C | C | S | N | N |
| 82 | 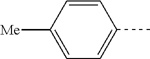 | H | bond | bond | N | C | N | CH | N |
| 83 | 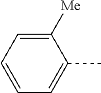 | H | bond | bond | N | C | N | CH | N |
| 84 | Ph | 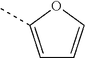 | bond | bond | N | C | N | CH | N |
| 85 | 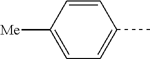 | H | bond | CH | C | C | N | N | N |
| 86 | 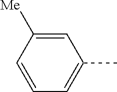 | H | bond | CH | C | C | N | N | N |
| 87 | 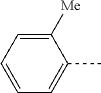 | H | bond | CH | C | C | N | N | N |
| 88 | 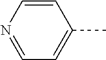 | H | bond | CH | C | C | N | N | N |
| 89 | 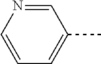 | H | bond | CH | C | C | N | N | N |
| 90 | 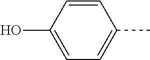 | H | bond | CH | C | C | N | N | N |
| 91 | 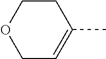 | H | bond | CH | C | C | N | N | N |
| 92 | Ph | OMe | bond | CH | C | C | N | N | N |
| 93 | 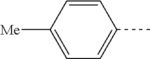 | H | bond | CH | C | C | N | N | CH |
| 94 | 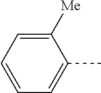 | H | bond | CH | C | C | N | N | CH |
| 95 | 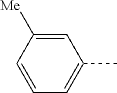 | H | bond | CH | C | C | N | N | CH |
| 96 | 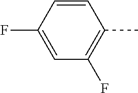 | H | bond | CH | C | C | N | N | CH |
| 97 | 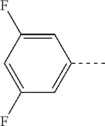 | H | bond | CH | C | C | N | N | CH |
| 98 | 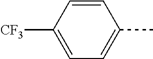 | H | bond | CH | C | C | N | N | CH |
| 99 | 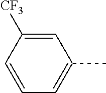 | H | bond | CH | C | C | N | N | CH |
| 100 | 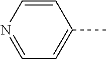 | H | bond | CH | C | C | N | N | CH |
| 101 | 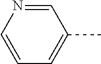 | H | bond | CH | C | C | N | N | CH |
| 102 | 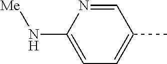 | H | bond | CH | C | C | N | N | CH |
| 103 | 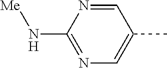 | H | bond | CH | C | C | N | N | CH |
| 104 | Ph | H | bond | bond | C | C | N | N | N—Me |
| 105 | 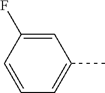 | H | bond | bond | C | C | N | N | N—Me |
| 106 | 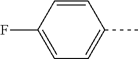 | H | bond | bond | C | C | N | N | N—Me |
| 107 | 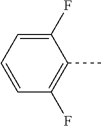 | H | bond | CH | C | C | N | N | N |
| 108 | 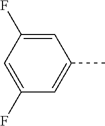 | H | bond | CH | C | C | N | N | N |
| 109 | 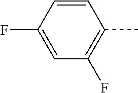 | H | bond | CH | C | C | N | N | N |
| 110 | 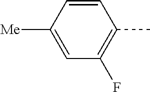 | H | bond | CH | C | C | N | N | N |
| 111 | 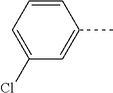 | H | bond | CH | C | C | N | N | N |
| 112 | 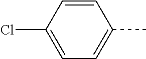 | H | bond | CH | C | C | N | N | N |
| 113 | 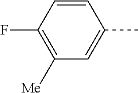 | H | bond | CH | C | C | N | N | N |
| 114 | 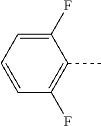 | H | bond | CH | C | C | N | N | CH |
| 115 | 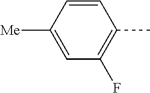 | H | bond | CH | C | C | N | N | CH |
| 116 | 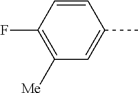 | H | bond | CH | C | C | N | N | CH |
| 117 | 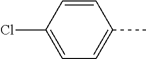 | H | bond | CH | C | C | N | N | CH |
| 118 | 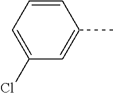 | H | bond | CH | C | C | N | N | CH |
| 119 | 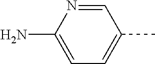 | H | bond | CH | C | C | N | N | CH |
| 120 | 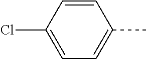 | H | bond | bond | C | C | S | N | N |
| 121 | 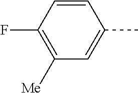 | H | bond | bond | C | C | S | N | N |

### Examples of Representative Pharmaceutical Compositions

In combination with commonly used solvents, excipients, auxiliary agents and carriers, the instant compounds may be processed into tablets, coated tablets, capsules, drip solutions, suppositories, injection and infusion preparations, and the like and may be therapeutically applied by the oral, rectal, parenteral, and additional routes. Representative pharmaceutical compositions according to the present invention follow:

(a) Tablets suitable for oral administration which contain the active ingredient, may be prepared by conventional tabletting techniques.

(b) For suppositories, any usual suppository base may be employed for incorporation thereinto by usual procedure of the active ingredient, such as a polyethyleneglycol which is a solid at normal room temperature but which melts at or about body temperature.

(c) For parenteral (including intravenous and subcutaneous) sterile solutions, the active ingredient together with conventional ingredients in usual amounts are employed, such as for example sodium chloride and double-distilled water q.s., according to conventional procedure, such as filtration, aseptic filling into ampoules or IV-drip bottles, and autoclaving for sterility.

Other suitable pharmaceutical compositions will be immediately apparent to one skilled in the art.

### Formulation Examples

The following examples are again given by way of illustration only and are not to be construed as limiting.

### Example 1

### Tablet Formulation

A suitable formulation for a tablet containing 10 milligrams of active ingredient is as follows:

| | mg |
|---|---|
| Active Ingredient | 10 |
| Lactose | 61 |
| Microcrystalline Cellulose | 25 |
| Talcum | 2 |
| Magnesium stearate | 1 |
| Colloidal silicon dioxide | 1 |

### Example 2

### Tablet Formulation

Another suitable formulation for a tablet containing 100 mg is as follows:

| | mg |
|---|---|
| Active Ingredient | 100 |
| Polyvinylpyrrolidone, crosslinked | 10 |
| Potato starch | 20 |
| Polyvinylpyrrolidone | 19 |
| Magnesium stearate | 1 |
| Microcrystalline Cellulose | 50 |
| Film coated and colored. | |
| The film coating material consists of: | |
| Hypromellose | 10 |
| Microcryst. Cellulose | 5 |
| Talcum | 5 |
| Polyethylene glycol | 2 |
| Color pigments | 5 |

### Example 3

### Capsule Formulation

A suitable formulation for a capsule containing 50 milligrams of active ingredient is as follows:

| | mg |
|---|---|
| Active Ingredient | 50 |
| Corn starch | 26 |
| Dibasic calcium phosphate | 50 |
| Talcum | 2 |
| Colloidal silicon dioxide | 2 |

filled in a gelatin capsule.

### Example 4

### Solution for Injection

A suitable formulation for an injectable solution is as follows:

| | | |
|---|---|---|
| Active Ingredient | mg | 10 |
| Sodium chloride | mg | q.s. |
| Water for Injection | mL | add 1.0 |

### Example 5

### Liquid Oral Formulation

A suitable formulation for 1 liter of a an oral solution containing 2 milligrams of active ingredient in one milliliter of the mixture is as follows:

| | mg |
|---|---|
| Active Ingredient | 2 |
| Saccharose | 250 |
| Glucose | 300 |
| Sorbitol | 150 |
| Orange flavor | 10 |
| Colorant | q.s. |
| Purified water | add 1000 mL |

### Example 6

### Liquid Oral Formulation

Another suitable formulation for 1 liter of a liquid mixture containing 20 milligrams of active ingredient in one milliliter of the mixture is as follows:

| | G |
|---|---|
| Active Ingredient | 20.00 |
| Tragacanth | 7.00 |
| Glycerol | 50.00 |
| Saccharose | 400.00 |
| Methyl paraben | 0.50 |
| Propylparaben | 0.05 |
| Black currant-flavor | 10.00 |
| Soluble Red color | 0.02 |
| Purified water | add 1000 mL |

### Example 7

### Liquid Oral Formulation

Another suitable formulation for 1 liter of a liquid mixture containing 2 milligrams of active ingredient in one milliliter of the mixture is as follows:

| | G |
|---|---|
| Active Ingredient | 2 |
| Saccharose | 400 |
| Bitter orange peel tincture | 20 |
| Sweet orange peel tincture | 15 |
| Purified water | add 1000 mL |

### Example 8

### Aerosol Formulation

180 g aerosol solution contain:

| | G |
|---|---|
| Active Ingredient | 10 |
| Oleic acid | 5 |
| Ethanol | 81 |
| Purified Water | 9 |
| Tetrafluoroethane | 75 |

15 mL of the solution are filled into aluminum aerosol cans, capped with a dosing valve, purged with 3.0 bar.

### Example 9

### TDS Formulation

100 g solution contain:

| | G |
|---|---|
| Active Ingredient | 10.0 |
| Ethanol | 57.5 |
| Propyleneglycol | 7.5 |
| Dimethylsulfoxide | 5.0 |
| Hydroxyethylcellulose | 0.4 |
| Purified water | 19.6 |

1.8 mL of the solution are placed on a fleece covered by an adhesive backing foil. The system is closed by a protective liner which will be removed before use.

### Example 10

### Nanoparticle Formulation

10 g of polybutylcyanoacrylate nanoparticles contain:

| | G |
|---|---|
| Active Ingredient | 1.00 |
| Poloxamer | 0.10 |
| Butylcyanoacrylate | 8.75 |
| Mannitol | 0.10 |
| Sodium chloride | 0.05 |

Polybutylcyanoacrylate nanoparticles are prepared by emulsion polymerization in a water/0.1 N HCl/ethanol mixture as polymerization medium. The nanoparticles in the suspension are finally lyophilized under vacuum.

### Example 11

### Suspension Formulation

1.0 g of the suspension contains the following:

| | g |
|---|---|
| Active Ingredient | 0.10 |
| Hypromellose | 0.01 |
| Purified water | Ad 1.0 g |

Hypromellose is dispersed in water homogeneously with a high speed mixer/blender. After about one hour of hydration time of the hypromellose, the active ingredient is blended homogeneously into the hypromellose solution. The viscosity of the suspension may be adjusted by the amount of hypromellose, resulting in a very stable suspension with a very slow tendency of particle sedimentation and particle agglomeration.

### Example 12

### Solution for Injection

1.0 mL of solution contain:

| | g |
|---|---|
| Active Ingredient | 0.05 |
| Mannitol | q.s. |
| DMSO | 0.10 |
| Water for injection | Ad 1.0 ml |

The active ingredient is dissolved in DMSO by stirring and heating (solution 1). The mannitol is dissolved in WFI (solution 2). After cooling down to room temperature solution 1 is mixed with solution 2 by continuous stirring. The solution is sterilized by filtration of by autoclaving.

### Pharmacology

The active principles of the present invention, and pharmaceutical compositions containing them and method of treating therewith, are characterized by unique and advantageous properties. The compounds and pharmaceutical compositions thereof exhibit, in standard accepted reliable test procedures, the following valuable properties and characteristics.

### Methods

Binding Assays for the Characterization of mGluR5 Antagonist Properties
[³H]MPEP (2-methyl-6-(phenylethynyl)pyridine) Binding to Transmembrane Allosteric Modulatory Sites of mGluR5 Receptors in Cortical Membranes

### Preparation of Rat Cortical Membranes:

Male Sprague-Dawley rats (200-250 g) are decapitated and their brains are removed rapidly. The cortex is dissected and homogenized in 20 volumes of ice-cold 0.32 M sucrose using a glass-Teflon homogenizer. The homogenate is centrifuged at 1000×g for 10 minutes. The pellet is discarded and the supernatant centrifuged at 20,000×g for 20 minutes. The resulting pellet is re-suspended in 20 volumes of distilled water and centrifuged for 20 minutes at 8000×g. Then the supernatant and the buffy coat are centrifuged at 48,000×g for 20 minutes in the presence of 50 mM Tris-HCl, pH 8.0. The pellet is then re-suspended and centrifuged two to three more times at 48,000×g for 20 minutes in the presence of 50 mM Tris-HCl, pH 8.0. All centrifugation steps are carried out at 4° C. After resuspension in 5 volumes of 50 mM Tris-HCl, pH 8.0, the membrane suspension is frozen rapidly at −80° C.

On the day of assay the membrane suspensions are thawed and washed four times by resuspension in 50 mM Tris-HCl, pH 8.0, and centrifugation at 48,000×g for 20 minutes and finally re-suspended in 50 mM Tris-HCl, pH 7.4. The amount of protein in the final membrane preparation (500-700 μg/ml) is determined according to the method of Lowry (Lowry O. H. et al. 1951. J. Biol. Chem. 193, 256-275).

### [³H]MPEP Assay

Incubations are started by adding [³H]-MPEP (50.2 Ci/mmol, 5 nM, Tocris, GB) to vials with 125-250 μg protein (total volume 0.25 ml) and various concentrations of the agents. Alternatively, assays are performed with [³H]-MMPEP (2-(3-methoxyphenylethynyl)-6-methylpyridine hydrochloride) as radioligand. The incubations are continued at room temperature for 60 minutes (equilibrium is achieved under the conditions used). Non-specific binding is defined by the addition of unlabeled MPEP (10 mM). Incubations are terminated using a Millipore filter system. The samples are rinsed twice with 4 mL of ice-cold assay buffer over glass fibre filters (Schleicher & Schuell, Germany) under a constant vacuum. Following separation and rinse, the filters are placed into scintillation liquid (5 mL Ultima Gold, Perkin Elmer, Germany) and radioactivity retained on the filters is determined with a conventional liquid scintillation counter (Canberra Packard, Germany).

### Characterization

Specific binding is extremely high i.e. normally >85% and essentially independent of buffer (Tris or HEPES both 50 mM) and pH (6.8-8.9). There is a clear saturable protein dependence and the chosen protein concentration used for subsequent assays (500-700 μg/ml) is within the linear portion of this dependence. Cold MPEP displaces hot ligand with an IC₅₀ of 11.2±0.64 nM. The K_{d} of [³H]-MPEP of 13.6 nM is determined by Scatchard analysis and used according to the Cheng Prussoff relationship to calculate the affinity of displacers as K_{d} values (IC₅₀ of cold MPEP equates to a Kᵢ of 8.2 nM). Bₘₐₓ is 0.56 pm/mg protein.

Functional Assay of mGluR5 Receptors
Cytosolic Calcium Studies with Stably Transfected Cells

Chinese hamster ovary cells (CHO-K1 cells), stably transfected for inducible expression of a human metabotropic glutamate receptor mGluR5, are seeded into black clear bottom 96 well plates at a density of 35.000 cells per well. The standard growth medium used (Dulbecco's modified Eagle Medium, DMEM plus L-proline) contains the appropriate inducer isopropyl-β-D-thiogalactopyranosid (IPTG) to achieve optimal receptor expression. One day after seeding the growth medium is exchanged for reconstituted Ca-Kit (Molecular Devices, USA) and incubated for one hour. Ca-Kit is reconstituted in an assay buffer containing 20 mM HEPES pH 7.4, glutamic-pyruvate transaminase, pyridoxal phosphate and sodium pyruvate in Hank's balanced salt solution (HBBS). Agonistic compounds to the receptor elicit increases in cytosolic calcium which can be measured as increases in fluorescence signals by use of a fluorescence imaging plate reader (Molecular Devices). To analyze their potency to negatively modulate the Ca-response test compounds, dissolved in a final DMSO concentration of 0.5%, are added on-line 5 minutes before the agonist to the receptor (L-quisqualic acid at a concentration giving ˜80% of the maximal signal). Similarly, for the characterization of positive allosteric modulators, a dilution series of the test compound is added online to 384 well plates containing the described dye loaded cells and after a preincubation time of 5 minutes stimulated by a basal cell concentration of the agonist L-glutamate giving about 20% of the maximal signal.

### Astrocyte Culture

Primary astrocyte cultures are prepared from cortices of newborn rats as described by Booher and Sensenbrenner (1972, Neurobiology 2(3):97-105). Briefly, Sprague-Dawley rat pups (2-4 d old) are decapitated and neocortices are dissected, disintegrated with a nylon filter (pore size 80 μm) and carefully triturated. The cell suspension is plated on poly-D-lysine pre-coated flasks (Costar, Netherlands) and cultivated in Dulbecco's Modified Eagle's Medium (DMEM, Invitrogen, Germany) supplemented with 10% foetal calf serum (FCS, Sigma, Germany), 4 mM glutamine and 50 kg/ml gentamycin (both Biochrom, Germany) at 37° C. in a humidified atmosphere of 5% CO₂/95% air for 7 days with exchanging the medium at day 2 and 6.

After 7 days in vitro (DIV), cells are shaken overnight at 250 rpm to remove oligodendrocytes and microglia. The next day, astrocytes are rinsed twice with CMF-PBS (calcium- and magnesium-free phosphate buffered saline, Biochrom, Germany), trypsinized and subplated on poly-D-lysine pre-coated 96-well plates (Greiner, Germany) at a density of 40,000 cells/well. 24 h after establishing the secondary culture the astrocytes are rinsed with PBS″ (phosphate buffered saline, Biochrom, Germany) and fed with astrocyte-defined medium (ADM) consisting of DMEM containing 1× G5-supplement (Invitrogen, Germany), 0.5 μg/ml heparan sulfate, and 1.5 μg/ml fibronectin (both Sigma, Germany) (Miller et al., (1993) Brain Res. 618(1):175-8). 3 days later the medium is exchanged and the cells incubated for another 2-3 days, so that at the time of experiments astrocytes are 14-15 DIV.

### Immunocytochemistry

Immunostaining is performed to confirm the presence of astrocytic markers such as the glial fibrillary acidic protein (GFAP) as well as to monitor the expression of mGluR5 receptors.

Cytosolic Calcium Studies with Astrocytes

The increase of cytosolic calcium after stimulation with the mGluR5 agonist L-quisqualate is measured using a fluorometric imaging plate reader (FLIPR) and the Ca-Kit (both Molecular Devices). Prior to addition of agonist or antagonist the medium is aspirated and cells are loaded for 2 h at RT with 150 μl of loading buffer consisting of Ca-sensitive dye reconstituted in sodium chloride (123 mM), potassium chloride (5.4 mM), magnesium chloride (0.8 mM), calcium chloride (1.8 mM), D-glucose (15 mM), and HEPES (20 mM), pH 7.3. Subsequently, plates are transferred to FLIPR to detect calcium increase with the addition of L-quisqualate (100 nM) measured as relative fluorescence units (RFU). If antagonists are tested, these compounds are pre-incubated for 10 minutes at RT before addition of the respective agonist.

For positive modulators, concentration-response curves for quisqualate are performed in the presence and absence of 10 uM modulator to determine the extent of potentiation/agonist potency increase. Thereafter, concentration-response curves for the positive modulator are performed in the presence of a fixed concentration of quisqualate showing the biggest window for potentiation (normally 10-30 nM).

### Data Analysis

The fluorescence signal increase after addition of agonist reflects the increase of cytosolic calcium. Inconsistencies in the amount of cells per well are normalised by using the spatial uniformity correction of the FLIPR operating software Screenworks. The mean of replicated temporal data (n=3-5) is calculated and used for graphical representation. For the evaluation of the pharmacology, the calcium changes in response to different concentrations of agonist or antagonist are determined using a maximum minus minimum (MaxMin) calculation.

All responses (RFU-values) are determined as percentage of control (=maximum response). EC₅₀ and IC₅₀ are calculated according the logistic equation using Prism 4.0 (GraphPad Software, USA). The compounds of the present invention have a potency (EC₅₀) within a range of about 0.5 nM to about 100 uM.

Results for representative compounds of the invention are shown in are shown in Tables A1 and A2.

TABLE A1
(Cytosolic Calcium studies with stably transfected cells)

| | | EC50 |
|---|---|---|
| Compound | Chemical Name | [uM] |
| Example 1 | 6-Phenylethynyl-pyrazolo[1,5-a]pyrimidine | 0.038 |
| Example 2 | 6-(3,5-Dichloro-phenylethynyl)-pyrazolo[1,5- | 0.52 |
| | a]pyrimidine | |
| Example 3 | 6-(3-Fluoro-phenylethynyl)-pyrazolo[1,5- | 0.021 |
| | a]pyrimidine | |
| Example 4 | 6-(4-Fluoro-phenylethynyl)-pyrazolo[1,5- | 0.12 |
| | a]pyrimidine | |
| Example 8 | 6-Cyclohex-1-enylethynyl-pyrazolo[1,5- | 0.023 |
| | a]pyrimidine | |
| Example 9 | 6-p-Tolylethynyl-pyrazolo[1,5-a]pyrimidine | 0.077 |
| Example 10 | 6-(3,6-Dihydro-2H-thiopyran-4-ylethynyl)- | 0.11 |
| | pyrazolo[1,5-a]pyrimidine | |
| Example 11 | 6-(3,5-Difluoro-phenylethynyl)-pyrazolo[1,5- | 0.20 |
| | a]pyrimidine | |
| Example 12 | 4-Pyrazolo[1,5-a]pyrimidin-6-ylethynyl-3,6- | 1.3 |
| | dihydro-2H-pyridine-1-carboxylic acid tert- | |
| | butyl ester | |
| Example 14 | 6-(3-Phenyl-prop-1-ynyl)-pyrazolo[1,5- | 0.12 |
| | a]pyrimidine | |
| Example 17 | Azepan-1-yl-(6-phenylethynyl-pyrazolo[1,5- | 0.26 |
| | a]pyrimidin-2-yl)-methanone | |
| Example 18 | (6-Phenylethynyl-pyrazolo[1,5-a]pyrimidin-2- | 0.34 |
| | yl)-(4-phenyl-piperidin-1-yl)-methanone | |
| Example 19 | (6-Phenylethynyl-pyrazolo[1,5-a]pyrimidin-2- | 0.61 |
| | yl)-pyrrolidin-1-yl-methanone | |
| Example 28 | 2-(4-Fluoro-phenyl)-6-phenylethynyl- | 0.093 |
| | pyrazolo[1,5-a]pyrimidine | |
| Example 29 | 6-Phenylethynyl-pyrazolo[1,5-a]pyridine | 0.078 |
| Example 30 | 6-Cyclohex-1-enylethynyl-pyrazolo[1,5- | 0.029 |
| | a]pyridine | |
| Example 32 | (6-Phenylethynyl-pyrazolo[1,5-a]pyridin-2-yl)- | 0.25 |
| | piperidin-1-yl-methanone | |
| Example 33 | 6-Phenylethynyl-[1,2,4]triazolo[1,5- | 0.13 |
| | a]pyrimidine | |
| Example 36 | (6-Phenylethynyl-[1,2,4]triazolo[1,5- | 0.94 |
| | a]pyrimidin-2-yl)-piperidin-1-yl-methanone | |
| Example 37 | 6-Phenylethynyl-[1,2,4]triazolo[1,5-a]pyridine | 0.11 |
| Example 38 | 6-Phenylethynyl-thiazolo[4,5-b]pyridine | 0.010 |
| Example 39 | 7-Phenylethynyl-pyrido[2,3-b]pyrazine | 0.035 |
| Example 40 | 7-Cyclohex-1-enylethynyl-pyrido[2,3- | 0.12 |
| | b]pyrazine | |
| Example 42 | 6-Phenylethynyl-oxazolo[4,5-b]pyridine | 0.47 |
| Example 43 | (6-Phenylethynyl-oxazolo[4,5-b]pyridin-2-yl)- | 0.15 |
| | piperidin-1-yl-methanone | |
| Example 44 | 6-(3-Fluoro-phenylethynyl)-thiazolo[4,5- | 0.023 |
| | b]pyridine | |
| Example 45 | 6-(2-Fluoro-phenylethynyl)-thiazolo[4,5- | 0.065 |
| | b]pyridine | |
| Example 46 | 6-(4-Fluoro-phenylethynyl)-thiazolo[4,5- | 0.058 |
| | b]pyridine | |
| Example 47 | 6-(3-Fluoro-phenylethynyl)-[1,2,4]triazolo[1,5- | 1.3 |
| | a]pyrimidine | |
| Example 48 | 6-(2-Fluoro-phenylethynyl)-[1,2,4]triazolo[1,5- | 1.0 |
| | a]pyrimidine | |
| Example 49 | 6-(4-Fluoro-phenylethynyl)-[1,2,4]triazolo[1,5- | 1.8 |
| | a]pyrimidine | |
| Example 53 | 6-(3-Fluoro-phenylethynyl)-[1,2,4]triazolo[1,5- | 0.43 |
| | a]pyridine | |
| Example 54 | 6-(2-Fluoro-phenylethynyl)-[1,2,4]triazolo[1,5- | 0.74 |
| | a]pyridine | |
| Example 55 | 6-(4-Fluoro-phenylethynyl)-[1,2,4]triazolo[1,5- | 0.64 |
| | a]pyridine | |
| Example 57 | [6-(3-Fluoro-phenylethynyl)-[1,2,4]triazolo[1,5- | 1.2 |
| | a]pyridin-2-yl]piperidin-1-yl-methanone | |
| Example 59 | [6-(4-Fluoro-phenylethynyl)-[1,2,4]triazolo[1,5- | 6.9 |
| | a]pyridin-2-yl]piperidin-1-yl-methanone | |
| Example 60 | 7-(3-Fluoro-phenylethynyl)-pyrido[2,3- | 0.1 |
| | b]pyrazine | |
| Example 61 | 7-(2-Fluoro-phenylethynyl)-pyrido[2,3- | 0.3 |
| | b]pyrazine | |
| Example 62 | 7-(4-Fluoro-phenylethynyl)-pyrido[2,3- | 0.55 |
| | b]pyrazine | |
| Example 67 | 3-(3-Fluoro-phenylethynyl)-[1,5]naphthyridine | 0.0068 |
| Example 68 | 3-(2-Fluoro-phenylethynyl)-[1,5]naphthyridine | 0.019 |
| Example 69 | 3-(4-Fluoro-phenylethynyl)-[1,5]naphthyridine | 0.0098 |
| Example 70 | 6-(4-Fluoro-phenylethynyl)-oxazolo[4,5- | 1.4 |
| | b]pyridine | |
| Example 71 | 6-Cyclohex-1-enylethynyl-oxazolo[4,5- | 0.0063 |
| | b]pyridine | |
| Example 72 | (6-Cyclohex-1-enylethynyl-oxazolo[4,5- | 0.33 |
| | b]pyridin-2-yl)-piperidin-1-yl-methanone | |
| Example 73 | 6-(m-Tolylethynyl)thiazolo[4,5-b]pyridine | 0.02 |
| Example 75 | 6-(o-Tolylethynyl)thiazolo[4,5-b]pyridine | 2.3 |
| Example 78 | 6-((2,6-Difluorophenyl)ethynyl)thiazolo[4,5- | 1.7 |
| | b]pyridine | |
| Example 79 | 6-((2,4-Difluorophenyl)ethynyl)thiazolo[4,5- | 0.31 |
| | b]pyridine | |
| Example 80 | 6-((3,5-Difluorophenyl)ethynyl)thiazolo[4,5- | 0.66 |
| | b]pyridine | |
| Example 81 | 6-Phenylethynyl-2-piperidin-1-yl-thiazolo[4,5- | 0.091 |
| | b]pyridine | |
| Example 84 | 2-Furan-2-yl-6-phenylethynyl- | 0.41 |
| | [1,2,4]triazolo[1,5-a]pyridine | |
| Example 87 | 7-(o-Tolylethynyl)-pyrido[2,3-b]pyrazine | 1.1 |
| Example 92 | 2-Methoxy-7-(phenylethynyl)pyrido[2,3- | 0.28 |
| | b]pyrazine | |
| Example 93 | 3-(p-Tolylethynyl)-[1,5]naphthyridine | 0.036 |
| Example 94 | 3-(o-Tolylethynyl)-[1,5]naphthyridine | 0.41 |
| Example 95 | 3-(m-Tolylethynyl)-[1,5]naphthyridine | 0.0055 |
| Example 100 | 3-(Pyridin-4-ylethynyl)-1,5-naphthyridine | 0.23 |
| Example 101 | 3-(Pyridin-3-ylethynyl)-1,5-naphthyridine | 0.47 |
| Example 104 | 3-Methyl-6-phenylethynyl-3H-imidazo[4,5- | 0.014 |
| | b]pyridine | |
| Example 105 | 6-(3-Fluoro-phenylethynyl)-3-methyl-3H- | 0.021 |
| | imidazo[4,5-b]pyridine | |
| Example 106 | 6-(4-Fluoro-phenylethynyl)-3-methyl-3H- | 0.057 |
| | imidazo[4,5-b]pyridine | |
| Example 107 | 7-((2,6-Difluorophenyl)ethynyl)pyrido[2,3- | 3.7 |
| | b]pyrazine | |
| Example 108 | 7-((3,5-Difluorophenyl)ethynyl)pyrido[2,3- | 4.1 |
| | b]pyrazine | |
| Example 109 | 7-((2,4-Difluorophenyl)ethynyl)pyrido[2,3- | 3.5 |
| | b]pyrazine | |
| Example 111 | 7-((3-Chlorophenyl)ethynyl)pyrido[2,3- | 0.12 |
| | b]pyrazine | |
| Example 115 | 3-((2-Fluoro-4-methylphenyl)ethynyl)-1,5- | 0.69 |
| | naphthyridine | |

TABLE A2
(Cytosolic Calcium studies with cultured rat astrocytes)

| | | EC50 |
|---|---|---|
| Compound | Chemical Name | [uM] |
| Example 1 | 6-Phenylethynyl-pyrazolo[1,5-a]pyrimidine | 0.089 |
| Example 3 | 6-(3-Fluoro-phenylethynyl)-pyrazolo[1,5- | 0.076 |
| | a]pyrimidine | |
| Example 4 | 6-(4-Fluoro-phenylethynyl)-pyrazolo[1,5- | 0.063 |
| | a]pyrimidine | |
| Example 8 | 6-Cyclohex-1-enylethynyl-pyrazolo[1,5- | 0.070 |
| | a]pyrimidine | |
| Example 10 | 6-(3,6-Dihydro-2H-thiopyran-4-ylethynyl)- | 0.050 |
| | pyrazolo[1,5-a]pyrimidine | |
| Example 32 | (6-Phenylethynyl-pyrazolo[1,5-a]pyridin-2-yl)- | 0.48 |
| | piperidin-1-yl-methanone | |
| Example 33 | 6-Phenylethynyl-[1,2,4]triazolo[1,5- | 0.23 |
| | a]pyrimidine | |
| Example 37 | 6-Phenylethynyl-[1,2,4]triazolo[1,5-a]pyridine | 0.11 |
| Example 38 | 6-Phenylethynyl-thiazolo[4,5-b]pyridine | 0.073 |
| Example 39 | 7-Phenylethynyl-pyrido[2,3-b]pyrazine | 0.10 |
| Example 40 | 7-Cyclohex-1-enylethynyl-pyrido[2,3- | 0.65 |
| | b]pyrazine | |
| Example 41 | 3-Phenylethynyl-[1,5]naphthyridine | 0.005 |
| Example 43 | (6-Phenylethynyl-oxazolo[4,5-b]pyridin-2-yl)- | 0.060 |
| | piperidin-1-yl-methanone | |
| Example 44 | 6-(3-Fluoro-phenylethynyl)-thiazolo[4,5- | 0.037 |
| | b]pyridine | |
| Example 45 | 6-(2-Fluoro-phenylethynyl)-thiazolo[4,5- | 0.056 |
| | b]pyridine | |
| Example 46 | 6-(4-Fluoro-phenylethynyl)-thiazolo[4,5- | 0.091 |
| | b]pyridine | |
| Example 47 | 6-(3-Fluoro-phenylethynyl)-[1,2,4]triazolo[1,5- | 1.5 |
| | a]pyrimidine | |
| Example 54 | 6-(2-Fluoro-phenylethynyl)-[1,2,4]triazolo[1,5- | 0.47 |
| | a]pyridine | |
| Example 55 | 6-(4-Fluoro-phenylethynyl)-[1,2,4]triazolo[1,5- | 0.40 |
| | a]pyridine | |
| Example 58 | [6-(2-Fluoro-phenylethynyl)-[1,2,4]triazolo[1,5- | 1.1 |
| | a]pyridin-2-yl]-piperidin-1-yl-methanone | |
| Example 60 | 7-(3-Fluoro-phenylethynyl)-pyrido[2,3- | 0.054 |
| | b]pyrazine | |
| Example 61 | 7-(2-Fluoro-phenylethynyl)-pyrido[2,3- | 0.19 |
| | b]pyrazine | |
| Example 62 | 7-(4-Fluoro-phenylethynyl)-pyrido[2,3- | 0.73 |
| | b]pyrazine | |
| Example 67 | 3-(3-Fluoro-phenylethynyl)-[1,5]naphthyridine | 0.0086 |
| Example 68 | 3-(2-Fluoro-phenylethynyl)-[1,5]naphthyridine | 0.024 |
| Example 69 | 3-(4-Fluoro-phenylethynyl)-[1,5]naphthyridine | 0.012 |
| Example 72 | (6-Cyclohex-1-enylethynyl-oxazolo[4,5- | 0.32 |
| | b]pyridin-2-yl)-piperidin-1-yl-methanone | |
| Example 73 | 6-(m-Tolylethynyl)thiazolo[4,5-b]pyridine | 0.014 |
| Example 75 | 6-(o-Tolylethynyl)thiazolo[4,5-b]pyridine | 0.53 |
| Example 79 | 6-((2,4-Difluorophenyl)ethynyl)thiazolo[4,5- | 0.22 |
| | b]pyridine | |
| Example 80 | 6-((3,5-Difluorophenyl)ethynyl)thiazolo[4,5- | 0.44 |
| | b]pyridine | |
| Example 90 | 4-(Pyrido[2,3-b]pyrazin-7-ylethynyl)phenol | 1.0 |
| Example 93 | 3-(p-Tolylethynyl)-[1,5]naphthyridine | 0.077 |
| Example 94 | 3-(o-Tolylethynyl)-[1,5]naphthyridine | 0.21 |
| Example 95 | 3-(m-Tolylethynyl)-[1,5]naphthyridine | 0.0023 |
| Example 100 | 3-(Pyridin-4-ylethynyl)-1,5-naphthyridine | 0.31 |

### Inhibition of Monoamine Oxidase MAO-B

The inhibition of the enzyme monoamine oxidase MAO-B is tested with an enzyme from recombinant source, namely of the human enzyme expressed in insect Hi5 cells. Following a pre-incubation of the test compounds with the enzyme for 15 minutes at 37° C. in the incubation buffer (100 mM potassium phosphate, pH 7.4), the enzymatic reaction with 50 uM kynuramine is carried out for 60 minutes. The reaction product 4-hydroxyquinoline is quantified by spectrofluorometry. The compound vehicle used for this assay is 1% DMSO.

The compounds of the present invention have a potency (IC₅₀) within a range of about 0.5 nM to about 100 uM.

Results for representative compounds of the invention are shown in are shown in Table A3.

TABLE A3
(MAO-B assay)

| | | IC50 |
|---|---|---|
| Compound | Chemical Name | [uM] |
| Example 46 | 6-(4-Fluoro-phenylethynyl)-thiazolo[4,5- | 0.54 |
| | b]pyridine | |
| Example 60 | 7-(3-Fluoro-phenylethynyl)-pyrido[2,3- | 0.089 |
| | b]pyrazine | |
| Example 61 | 7-(2-Fluoro-phenylethynyl)-pyrido[2,3- | 0.037 |
| | b]pyrazine | |
| Example 81 | 6-Phenylethynyl-2-piperidin-1-yl-thiazolo[4,5- | 0.52 |
| | b]pyridine | |

In conclusion, from the foregoing, it is apparent that the present invention provides novel and valuable applications and uses of the compounds of the present invention, which compounds comprise the active principle according to the present invention, as well as novel pharmaceutical compositions thereof and methods of preparation thereof and of treating therewith.

The high order of activity of the active agent of the present invention and compositions thereof, as evidenced by the tests reported, is indicative of utility based on its valuable activity in human beings as well as in lower animals. Clinical evaluation in human beings has not been completed. It will be clearly understood that the distribution and marketing of any compound or composition falling within the scope of the present invention for use in human beings will of course have to be predicated upon prior approval by governmental agencies which are responsible for and authorized to pass judgment on such questions.

The instant compounds of Formula I represent a novel class of mGluR5 modulators. In view of their potency, they will be useful therapeutics in a wide range of disorders, in particular CNS disorders, which involve excessive glutamate induced excitation.

These compounds accordingly find application in the treatment of the disorders of a living animal body, especially a human, as listed earlier in the description.

These compounds also find application in the treatment of indications in a living animal body, especially a human, wherein a particular condition does not necessarily exist but wherein a particular physiological parameter may be improved through administration of the instant compounds, including cognitive enhancement.

Neuroprotection as well as cognitive enhancement may also be achieved by administration of the instant compounds in combination with NMDA receptor antagonists like Memantine and Neramexane.

The method-of-treating a living animal body with a compound of the invention, for the inhibition of progression or alleviation of the selected ailment therein, is as previously stated by any normally-accepted pharmaceutical route, employing the selected dosage which is effective in the alleviation of the particular ailment desired to be alleviated. Use of the compounds of the present invention in the manufacture of a medicament for the treatment of a living animal for inhibition of progression or alleviation of selected ailments or conditions, particularly ailments or conditions susceptible to treatment with a Group I mGluR modulator is carried out in the usual manner comprising the step of admixing an effective amount of a compound of the invention with a pharmaceutically-acceptable diluent, excipient, or carrier, and the method-of-treating, pharmaceutical compositions, and use of a compound of the present invention in the manufacture of a medicament.

Representative pharmaceutical compositions prepared by admixing the active ingredient with a suitable pharmaceutically-acceptable excipient, diluent, or carrier, include tablets, capsules, solutions for injection, liquid oral formulations, aerosol formulations, TDS formulations, and nanoparticle formulations, thus to produce medicaments for oral, injectable, or dermal use, also in accord with the foregoing.

The present invention is not to be limited in scope by the specific embodiments described herein. Indeed, various modifications of the invention in addition to those described herein will become apparent to those skilled in the art from the foregoing description.

All patents, applications, publications, test methods, literature, and other materials cited herein are hereby incorporated by reference.

1-18. (canceled)

19. A compound selected from those of Formula I: 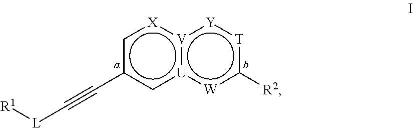
wherein.
L represents a bond or CH₂;
T represents a bond or CH;
U and V represent C or N;
W represents N, O, or S;
X represents CH or N;
Y represents CH, N, or N—R⁵, wherein R⁵ represents C₁₋₆alkyl;
it being understood that the valency of the atoms is respected and that the variables a and b represent the points of attachment for the 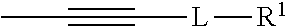
moiety and the R² substituent, respectively;
R¹ represents aryl, heteroaryl, cycloC₃₋₁₂alkyl, cycloC₃₋₁₂alkenyl, or heterocyclyl;
R² represents hydrogen, C₁₋₆alkyl, C₁₋₆alkoxycarbonyl, cycloC₃₋₁₂alkoxycarbonyl, aryl, heteroaryl, C₁₋₆alkoxy, —NR³R⁴, or —C(O)NR³R⁴, wherein R³ and R⁴, which may be the same or different, each independently represent hydrogen, C₁₋₆alkyl, or cycloC₃₋₁₂alkyl, or R³ and R⁴, together with the nitrogen atom to which they are attached, represent a 5-, 6-, or 7-membered ring which may be saturated or unsaturated, wherein the ring in addition to the nitrogen atom may contain, an additional heteroatom selected from sulfur, oxygen and nitrogen and/or be optionally fused to a benzene ring, and wherein the ring may be optionally substituted by one or more substituents selected from C₁₋₆alkyl, halogen, trifluoromethyl, C₁₋₆alkoxy, hydroxy, cyano, oxo, and phenyl;
wherein the term “aryl” means phenyl or naphthyl, wherein the phenyl or naphthyl, group is optionally substituted by one or more substituents, which may be the same or different, selected independently from halogen, trifluoromethyl, trifluoromethoxy, C₁₋₆alkyl, hydroxyC₁₋₆alkyl, C₂₋₆alkenyl, C₁₋₆alkoxy, C₁₋₆alkoxyC₁₋₆alkyl, amino, hydroxy, nitro, cyano, formyl, cyanomethyl, C₁₋₆alkoxycarbonyl, C₁₋₆alkylcarbonyloxy, C₁₋₆alkylcarbonyloxyC₁₋₆alkyl, C₁₋₆alkylamino, di-(C₁₋₆alkyl)amino, C₁₋₆alkylcarbonylamino, phenylcarbonylamino, aminocarbonyl, N—C₁₋₆alkylaminocarbonyl, C₁₋₆alkylaminocarbonyl, pyrrolidinyl, piperidinyl, morpholinyl, piperazinyl, cycloC₃₋₁₂alkyl and optionally C₁₋₆alkylenedioxy;
the term “heteroaryl” means an aromatic 5-6 membered ring containing from one to four heteroatoms selected from oxygen, sulfur and nitrogen, or a bicyclic group comprising a 5-6 membered ring containing from one to four heteroatoms selected from oxygen, sulfur and nitrogen fused with a benzene ring or a 5-6 membered ring containing from one to four heteroatoms selected from oxygen, sulfur and nitrogen, wherein the heteroaryl group may be optionally substituted by one or more substituents, which may be the same or different, selected independently from halogen, trifluoromethyl, trifluoromethoxy, C₁₋₆alkyl, hydroxyC₁₋₆alkyl, C₂₋₆alkenyl, C₁₋₆alkoxy, amino, hydroxy, nitro, cyano, C₁₋₆alkoxycarbonyl, C₁₋₆alkoxycarbonyloxy, C₁₋₆alkylamino, and di-(C₁₋₆alkyl)amino, C₁₋₆alkylcarbonylamino, aminocarbonyl, N—C₁₋₆alkylaminocarbonyl, di-N,N—C₁₋₆alkylaminocarbonyl, pyrrolidinyl, piperidinyl, morpholinyl, cycloC₃₋₁₂alkyl, C₁₋₆alkylenedioxy and aryl;
and optical isomers, prodrugs, pharmaceutically acceptable salts, hydrates, solvates, and polymorphs thereof;
it being understood that:
if T represents CH, then W and X each represent N;
if T represents a bond, then at least one of U or X. represents N;
if T represents a bond and W, U, and X all represent N, then R¹ may not represent cycloC₃₋₁₂alkyl or saturated heterocyclyl;
R¹ may not represent quinazoline;
and
the compound of Formula I may not represent
6-[2-(3-fluorophenylethynyl)]-[1,2,4]triazolo[1,5-a]pyridine,
6-[2-(3-nitrophenylethynyl)]-[1,2,4]triazolo[1,5-a]pyridine,
6-[2-(3-methylphenylethynyl)]-[1,2,4]triazolo[1,5-a]pyridine,
6-[2-(4-chlorophenylethynyl)]-[1,2,4]triazolo[1,5-a]pyridine,
6-[2-(4-fluorophenylethynyl)]-[1,2,4]triazolo[1,5-a]pyridine,
6-[2-(4-methylphenylethynyl)]-[1,2,4]triazolo[1,5-a]pyridine,
6-[2-(3,4-difluorophenylethynyl)]-[1,2,4]triazolo[1,5-a]pyridine,
6-[2-(2-chlorophenylethynyl)]-[1,2,4]-triazolo[1,5-a]pyridine,
6-[2-(3-chlorophenylethynyl)]-[1,2,4]triazolo[1,5-a]pyridine,
6-[2-(4-methyl-2-thiazolyl)ethynyl]-[1,2,4]triazolo[1,5-a]pyridine, or
6-[2-(6-methyl-2-pyridinyl)ethynyl]-[1,2,4]triazolo[1,5-a]pyridine.

20. The compound as claimed in claim 19, which is selected from those of Formula IA:

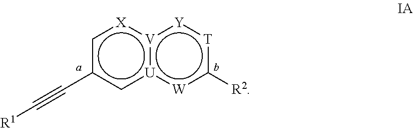

21. The compound as claimed in claim 19, which is selected from those of Formula IB:

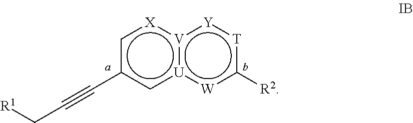

22. The compound as claimed in any of claim 19, wherein the ring represented by 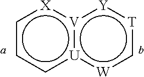
is selected from:

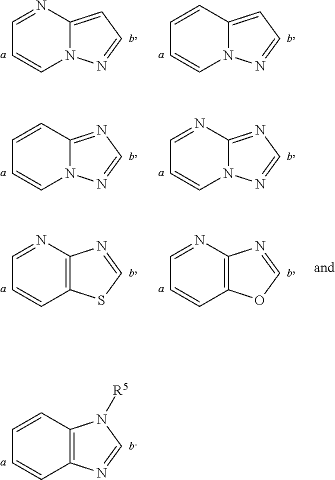

23. The compound as claimed in claim 22, wherein R⁵ represents methyl.

24. The compound as claimed in claim 19, wherein the ring represented by 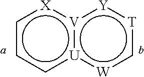
is selected from:

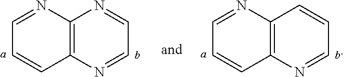

25. The compound as claimed in claim 19, wherein R¹ represents aryl, heteroaryl, cycloC₃₋₁₂alkenyl, or heterocyclyl.

26. The compound as claimed in claim 25, wherein R¹ represents phenyl which is optionally substituted by one or more substituents selected from halogen, C₁₋₆alkyl, hydroxy, and trifluoromethyl; thiophenyl which is optionally substituted by one or more C₁₋₆alkyl groups; cyclohexenyl; dihydrothiopyran; dihydropyridine which may be optionally substituted by one or more C₁₋₆alkoxycarbonyl groups; dihydropyran; pyridine which may be optionally substituted by one or more substituents selected from amino and C₁₋₆alkylamino; or pyrimidine which may be optionally substituted by one or more C₁₋₆alkylamino groups.

27. The compound as claimed in claim 19, wherein R² represents hydrogen, aryl, heteroaryl, C₁₋₆alkoxy, or —NR³R⁴, or —C(O)NR³R⁴, wherein R³ and together with the nitrogen atom to which they are attached, represent a 5-, 6-, or 7-membered ring which may be saturated or unsaturated, wherein the ring in addition to the nitrogen atom may contain an additional heteroatom selected from sulfur, oxygen and nitrogen and/or be optionally fused to a benzene ring, and wherein the ring may be optionally substituted by one or more substituents selected from C₁₋₆alkyl, hydroxy, oxo, and phenyl.

28. The compound as claimed in claim 27, wherein R² represents hydrogen, phenyl which is optionally substituted by one or more halogen atoms, piperidino, methoxy, furanyl, or —C(O)NR³R⁴, wherein R³ and R⁴ together with the nitrogen atom to which they are attached, represent a ring selected from morpholine, piperidine, pyrrolidine, azepine, and 1,3-dihydro-isoindole, wherein the ring may be optionally substituted by one or more substituents selected from methyl, hydroxy, oxo, and phenyl.

29. A compound as claimed in claim 19, which is selected from:
6-Phenylethynyl-pyrazolo[1,5-a]pyrimidine,
6-(3,5-Dichloro-phenylethynyl)-pyrazolo[1,5-a]pyrimidine,
6-(3-Fluoro-phenylethynyl)-pyrazolo[1,5-a]pyrimidine,
6-(4-Fluoro-phenylethynyl)-pyrazolo[1,5-a]pyrimidine,
6-(2-Fluoro-phenylethynyl)-pyrazolo[1,5-a]pyrimidine,
6-Thiophen-3-ylethynyl-pyrazolo[1,5-a]pyrimidine,
6-(3-Methyl-thiophen-2-ylethynyl)-pyrazolo[1,5-a]pyrimidine,
6-Cyclohex-1-enylethynyl-pyrazolo[1,5-a]pyrimidine,
6-p-Tolylethynyl-pyrazolo[1,5-a]pyrimidine,
6-(3,6-Dihydro-2H-thiopyran-4-ylethynyl)-pyrazolo[1,5-a]pyrimidine,
6-(3,5-Difluoro-phenylethynyl)-pyrazolo[1,5-a]pyrimidine,
4-Pyrazolo[1,5-a]pyrimidin-6-ylethynyl-3,6-dihydro-2H-pyridine-1-carboxylic acid tert-butyl ester,
6-Thiophen-2-ylethynyl-pyrazolo[1,5-a]pyrimidine,
6-(3-Phenyl-prop-1-ynyl)-pyrazolo[1,5-a]pyrimidine,
Morpholin-4-yl-(6-phenylethynyl-pyrazolo[1,5-a]pyrimidin-2-yl)-methanone,
(6-Phenylethynyl-pyrazolo[1,5-a]pyrimidin-2-yl)-piperidin-1-yl-methanone,
Azepan-1-yl-(6-phenylethynyl-pyrazolo[1,5-a]pyrimidin-2-yl)-methanone,
(6-Phenylethynyl-pyrazolo[1,5-a]pyrimidin-2-yl)-(4-phenyl-piperidin-1-yl)-methanone,
(6-Phenylethynyl-pyrazolo[1,5-a]pyrimidin-2-yl)-pyrrolidin-1-yl-methanone,
(1,3-Dihydro-isoindol-2-yl)-(6-phenylethynyl-pyrazolo[1,5-a]pyrimidin-2-yl)-methanone,
1-(6-Phenylethynyl-pyrazolo[1,5-a]pyrimidine-2-carbonyl)-piperidin-4-one,
4-[2-(Piperidine-1-carbonyl)-pyrazolo[1,5-a]pyrimidin-6-ylethynyl]-3,6-dihydro-2H-pyridine-1-carboxylic acid tert-butyl ester,
(4-Hydroxy-4-methyl-piperidin-1-yl)-(6-phenylethynyl-pyrazolo[1,5-a]pyrimidin-2-yl)-methanone,
(4-Hydroxy-piperidin-1-yl)-(6-phenylethynyl-pyrazolo[1,5-a]pyrimidin-2-yl)-methanone,
(1-Methyl-3,4-dihydro-1H-isoquinolin-2-yl)-(6-phenylethynyl-pyrazolo[1,5-a]pyrimidin-2-yl)-methanone,
6-Phenylethynyl-pyrazolo[1,5-a]pyrimidine-2-carboxylic acid cyclohexylamide,
6-Phenylethynyl-pyrazolo[1,5-a]pyrimidine-2-carboxylic acid cyclopentylamide,
2-(4-Fluoro-phenyl)-6-phenylethynyl-pyrazolo[1,5-a]pyrimidine,
6-Phenylethynyl-pyrazolo[1,5-a]pyridine,
6-Cyclohex-1-enylethynyl-pyrazolo[1,5-a]pyridine,
6-p-Tolylethynyl-pyrazolo[1,5-a]pyridine,
(6-Phenylethynyl-pyrazolo[1,5-a]pyridin-2-yl)-piperidin-1-yl-methanone,
6-Phenylethynyl-[1,2,4]triazolo[1,5-a]pyrimidine,
6-Thiophen-2-ylethynyl-[1,2,4]triazolo[1,5-a]pyrimidine,
6-p-Tolylethynyl-[1,2,4]triazolo[1,5-a]pyrimidine,
(6-Phenylethynyl-[1,2,4]triazolo[1,5-a]pyrimidin-2-yl)-piperidin-1-yl-methanone,
6-Phenylethynyl-[1,2,4]triazolo[1,5-a]pyridine,
6-Phenylethynyl-thiazolo[4,5-b]pyridine,
7-Phenylethynyl-pyrido[2,3-b]pyrazine,
7-Cyclohex-1-enylethynyl-pyrido[2,3-b]pyrazine,
3-Phenylethynyl-[1,5]naphthyridine,
6-Phenylethynyl-oxazolo[4,5-b]pyridine,
(6-Phenylethynyl-oxazolo[4,5-b]pyridin-2-yl)-piperidin-1-yl-methanone,
6-(3-Fluoro-phenylethynyl)-thiazolo[4,5-b]pyridine,
6-(2-Fluoro-phenylethynyl)-thiazolo[4,5-b]pyridine,
6-(4-Fluoro-phenylethynyl)-thiazolo[4,5-b]pyridine,
6-(3-Fluoro-phenylethynyl)-[1,2,4]triazolo[1,5-a]pyrimidine,
6-(2-Fluoro-phenylethynyl)-[1,2,4]triazolo[1,5-a]pyrimidine,
6-(4-Fluoro-phenylethynyl)-[1,2,4]triazolo[1,5-a]pyrimidine,
[6-(3-Fluoro-phenylethynyl)-[1,2,4]triazolo[1,5-a]pyrimidin-2-yl]-piperidin-1-yl-methanone,
[6-(2-Fluoro-phenylethynyl)-[1,2,4]triazolo[1,5-a]pyrimidin-2-yl]-piperidin-1-yl-methanone,
[6-(4-Fluoro-phenylethynyl)-[1,2,4]triazolo[1,5-a]pyrimidin-2-yl]piperidin-1-yl-methanone,
6-(3-Fluoro-phenylethynyl)-[1,2,4]triazolo[1,5-a]pyridine,
6-(2-Fluoro-phenylethynyl)-[1,2,4]triazolo[1,5-a]pyridine,
6-(4-Fluoro-phenylethynyl)-[1,2,4]triazolo[1,5-a]pyridine,
[6-Phenylethynyl-[1,2,4]triazolo[1,5-a]pyridin-2-yl]-piperidin-1-yl-methanone,
[6-(3-Fluoro-phenylethynyl)-[1,2,4]triazolo[1,5-a]pyridin-2-yl]-piperidin-1-yl-methanone,
[6-(2-Fluoro-phenylethynyl)-[1,2,4]triazolo[1,5-a]pyridin-2-yl]-piperidin-1-yl-methanone,
[6-(4-Fluoro-phenylethynyl)-[1,2,4]triazolo[1,5-a]pyridin-2-yl]-piperidin-1-yl-methanone,
7-(3-Fluoro-phenylethynyl)-pyrido[2,3-b]pyrazine,
7-(2-Fluoro-phenylethynyl)-pyrido[2,3-b]pyrazine,
7-(4-Fluoro-phenylethynyl)-pyrido[2,3-b]pyrazine,
[7-phenylethynyl-pyrido[2,3-b]pyrazin-3-yl]-piperidin-1-yl-methanone,
[7-(3-Fluoro-phenylethynyl)-pyrido[2,3-b]pyrazin-3-yl]-piperidin-1-yl-methanone,
[7-(2-Fluoro-phenylethynyl)-pyrido[2,3-b]pyrazin-3-yl]-piperidin-1-yl-methanone,
[7-(4-Fluoro-phenylethynyl)-pyrido[2,3-b]pyrazin-3-yl]-piperidin-1-yl-methanone,
3-(3-Fluoro-phenylethynyl)-[1,5]naphthyridine,
3-(2-Fluoro-phenylethynyl)-[1,5]naphthyridine,
3-(4-Fluoro-phenylethynyl)-[1,5]naphthyridine,
6-(4-Fluoro-phenylethynyl)-oxazolo[4,5-b]pyridine,
6-Cyclohex-1-ethylethynyl-oxazolo[4,5-b]pyridine,
(6-Cyclohex-1-enylethynyl-oxazolo[4,5-b]pyridin-2-yl)-piperidin-1-yl-methanone,
6-(m-Tolylethynyl)thiazolo[4,5-b]pyridine,
6-(p-Tolylethynyl)thiazolo[4,5-b]pyridine,
6-(o-Tolylethynyl)thiazolo[4,5-b]pyridine,
6-(Pyridin-4-ylethynyl)thiazolo[4,5-b]pyridine,
6-(Pyridin-3-ylethynyl)thiazolo[4,5-b]pyridine,
6-((2,6-Difluorophenyl)ethynyl)thiazolo[4,5-b]pyridine,
6-((2,4-Difluorophenyl)ethynyl)thiazolo[4,5-b]pyridine,
6-((3,5-Difluorophenyl)ethynyl)thiazolo[4,5-b]pyridine,
6-Phenylethynyl-2-piperidin-1-yl-thiazolo[4,5-b]pyridine,
6-(p-Tolylethynyl)-[1,2,4]triazolo[1,5-a]pyridine,
6-(o-Tolylethynyl)[1,2,4]triazolo[1,5-a]pyridine,
2-Furan-2-yl-6-phenylethynyl-[1,2,4]triazolo[1,5-a]pyridine,
7-(p-Tolylethynyl)-pyrido[2,3-b]pyrazine,
7-(m-Tolylethynyl)-pyrido[2,3-b]pyrazine,
7-(o-Tolylethynyl)-pyrido[2,3-b]pyrazine,
7-(Pyridin-4-ylethynyl)pyrido[2,3-b]pyrazine,
7-(Pyridin-3-ylethynyl)pyrido[2,3-b]pyrazine,
4-(Pyrido[2,3-b]pyrazin-7-ylethynyl)phenol,
7-((3,6-Dihydro-2H-pyran-4-yl)ethynyl)pyrido[2,3-b]pyrazine,
2-Methoxy-7-(phenylethynyl)pyrido[2,3-b]pyrazine,
3-(p-Tolylethynyl)-[1,5]naphthyridine,
3-(o-Tolylethynyl)-[1,5]naphthyridine,
3-(m-Tolylethynyl)-[1,5]naphthyridine,
3-(2,4-Difluoro-phenylethynyl)-[1,5]naphthyridine,
3-(3,5-Difluoro-phenylethynyl)-[1,5]naphthyridine,
3-((4-(Trifluoromethyl)phenyl)ethynyl)-1,5-naphthyridine,
3-((3-(Trifluoromethyl)phenyl)ethynyl)-1,5-naphthyridine,
3-(Pyridin-4-ylethynyl)-1,5-naphthyridine,
3-(Pyridin-3-ylethynyl)-1,5-naphthyridine,
5-((1,5-Naphthyridin-3-yl)ethynyl)-N-methylpyridin-2-amine,
5-((1,5-Naphthyridin-3-yl)ethynyl)-N-methylpyrimidin-2-amine,
3-Methyl-6-phenylethynyl-3H-imidazo[4,5-b]pyridine,
6-(3-Fluoro-phenylethynyl)-3-methyl-3H-imidazo[4,5-b]pyridine,
6-(4-Fluoro-phenylethylnyl)-3-methyl-3H-imidazo[4,5-b]pyridine, and
optical isomers, prodrugs, pharmaceutically acceptable salts, hydrates, solvates, and polymorphs thereof.

30. A pharmaceutical composition comprising a compound as claimed in claim 19, together with one or more pharmaceutically acceptable excipients.

31. A method for treating or preventing abnormal glutamate neurotransmission in a subject in need thereof comprising administering a therapeutically effective amount of a compound as claimed in claims 19.

32. A method for treating or preventing a condition or disease selected from Alzheimer's disease, Creutzfeld-Jakob's syndrome/disease, bovine spongiform encephalopathy (BSE), prion related infections, diseases involving mitochondrial dysfunction, diseases involving β-amyloid and/or tauopathy, Down's syndrome, hepatic encephalopathy, Huntington's disease, motor neuron diseases, amyotrophic lateral sclerosis (ALS), olivoponto-cerebellar atrophy, post-operative cognitive deficit (POCD), systemic lupus erythematosus, systemic clerosis, Sjogren's syndrome, Neuronal Ceroid Lipofuscinosis, neurodegenerative cerebellar ataxias, Parkinson's disease, Parkinson's dementia, mild cognitive impairment, cognitive deficits in various forms of mild cognitive impairment, cognitive deficits in various forms of dementia, dementia pugilistica, vascular and frontal lobe dementia, cognitive impairment, learning impairment, eye injuries, eye diseases, eye disorders, glaucoma, retinopathy, macular degeneration, head or brain or spinal cord injuries, head or brain or spinal cord trauma, trauma, hypoglycaemia, hypoxia, perinatal hypoxia, ischaemia, ischaemia resulting from cardiac arrest or stroke or bypass operations or transplants, convulsions, epileptic convulsions, epilepsy, temporal lobe epilepsy, myoclonic epilepsy, inner ear insult, inner ear insult in tinnitus, tinnitus, sound- or drug-induced inner ear insult, sound- or drug-induced tinnitus, L-dopa-induced dykinesias, L-dopa-induced dykinesias in Parkinson's disease therapy, dyskinesias, dyskinesia in Huntington's disease, drug induced dyskinesias, neuroleptic-induced dyskinesias, haloperidol-induced dyskinesias, dopaminomimetic-induced dyskinesias, chorea, Huntington's chorea, athetosis, dystonia, stereotypy, ballism, tardive dyskinesias, tic disorder, torticollis spasmodicus, blepharospasm, focal and generalized dystonia, nystagmus, hereditary cerebellar ataxias, corticobasal degeneration, tremor, essential tremor, abuse, addiction, nicotine addiction, nicotine abuse, alcohol addiction, alcohol abuse, opiate addiction, opiate abuse, cocaine addiction, cocaine abuse, amphetamine addiction, amphetamine abuse, anxiety disorders, panic disorders, anxiety and panic disorders, social anxiety disorder (SAD), attention deficit hyperactivity disorder (ADHD), attention deficit syndrome (ADS), restless leg syndrome (RLS), hyperactivity in children, autism, dementia, dementia in Alzheimer's disease, dementia in Korsakoff syndrome, Korsakoff syndrome, vascular dementia, dementia related to HIV infections, HIV-1 encephalopathy, AIDS encephlopathy, AIDS dementia complex, AIDS-related dementia, major depressive disorder, major depression, depression, depression resulting from Borna virus infection, major depression resulting from Borna virus infection, bipolar manic-depressive disorder, drug tolerance, drug tolerance to opioids, movement disorders, fragile-X syndrome, irritable bowel syndrome (IBS), migraine, multiple sclerosis (MS), muscle spasms, pain, chronic pain, acute pain, inflammatory pain, neuropathic pain, diabetic neuropathic pain (DNP), pain related to rheumatic arthritis, allodynia, hyperalgesia, nociceptive pain, cancer pain, posttraumatic stress disorder (PTSD), schizophrenia, positive or cognitive or negative symptoms of schizophrenia, spasticity, Tourette's syndrome, urinary incontinence, vomiting, pruritic conditions, pruritis, sleep disorders, micturition disorders, neuromuscular disorder in the lower urinary tract, gastroesophageal reflux disease (GERD), gastrointestinal dysfunction, lower esophageal sphincter (LES) disease, functional gastrointestinal disorders, dyspepsia, regurgitation, respiratory tract infection, bulimia nervosa, chronic laryngitis, asthma, reflux-related asthma, lung disease, eating disorders, obesity, obesity-related disorders, obesity abuse, food addiction, binge eating disorders, agoraphobia, generalized anxiety disorder, obsessive-compulsive disorder, panic disorder, posttraumatic stress disorder, social phobia, phobic disorders, substance-induced anxiety disorder, delusional disorder, schizoaffective disorder, schizophreniform disorder, substance-induced psychotic disorder, or delirium; inhibition of tumour cell growth, migration, invasion, adhesion and toxicity in the peripheral tissues, peripheral nervous system and CNS; neoplasia, hyperplasia, dysplasia, cancer, carcinoma, sarcoma, oral cancer, squamous cell carcinoma (SCC), oral squamous cell carcinoma (SCC), lung cancer, lung adenocarcinoma, breast cancer, prostate cancer, gastric cancer, liver cancer, colon cancer, colorectal carcinoma, rhabdomyosarcoma, brain tumour, tumour of a nerve tissue, glioma, malignant glioma, astroglioma, neuroglioma, neuroblastoma, glioblastoma, medulloblastoma, cancer of skin cells, melanoma, malignant melanoma, epithelial neoplasm, lymphoma, myeloma, Hodgkin's disease, Burkitt's lymphoma, leukemia, thymoma, tumours, diabetes, hyperammonemia and liver failure and sleep disturbances in a subject in need thereof administering a therapeutically effective amount of a compound as claimed in claim 19.

33. A pharmaceutical composition comprising a combination of at least one compound as claimed in claim 19 and at least one NMDA receptor antagonist, together with one or more pharmaceutically acceptable excipients.
